(19) United States
(12) Patent Application Publication
(10) Pub. No.: US 2020/0270588 A1
(43) Pub. Date: Aug. 27, 2020

(54) GLUCOSYL TRANSFERASE POLYPEPTIDES AND METHODS OF USE

(71) Applicant: Syngenta Participations AG, Basel (CH)

(72) Inventors: Timothy Robert Hawkes, Bracknell, Berkshire (GB); Richard Dale, Bracknell, Berkshire (GB); John Paul Evans, Hertfordshire (GB); Michael Phillip Langford, Bracknell, Berkshire (GB); Sabrina Guillemer, Nimes (FR); Shujie Dong, Research Triangle Park, NC (US); Qiudeng Que, Research Triangle Park, NC (US); Zhongying Chen, Research Triangle Park, NC (US); Yingping Lucy Qin, Research Triangle Park, NC (US)

(73) Assignee: Syngenta Participations AG, Basel (CH)

(21) Appl. No.: 16/610,221

(22) PCT Filed: May 4, 2018
(86) PCT No.: PCT/US2018/031038

§ 371 Date: Nov. 1, 2019

Related U.S. Application Data
(60) Provisional application No. 62/507,255, filed on May 17, 2017.

(51) Int. Cl. C12N 9/10; C12N 15/113; C12N 15/82

(52) U.S. Cl. CPC C12N 9/1051; C12N 15/1137; C12N 15/8213; C12N 15/8274; C12Y 204/01027

(57) ABSTRACT

Compositions and methods for conferring herbicide resistance or tolerance upon plants towards certain classes of herbicide are provided. In particular these are amine, alcohol and aminal herbicides. The compositions include nucleotide and amino acid sequences for wild-type and mutant glucosyl transferase polypeptides. The polypeptides of the invention are mutant or wild type glucosyl transferases that are capable of catalyzing the transfer of glucose to certain herbicidal structures and that, thereby, confer resistance or tolerance in plants to amine, alcohol and aminal PSII herbicides. Particularly, polypeptides of the invention include mutant or wild-type bx-type UDP glucosyl transferases.

### FIELD OF THE INVENTION

The present invention relates to glucosyl transferase polypeptides that confer herbicide resistance or tolerance to plants and the nucleic acid sequences that encode them. Methods of the invention relate to the production and use of plants that express glucosyl transferase polypeptides.

### BACKGROUND

Glucosyl transferases are enzymes that are found ubiquitously in nature and that catalyze glyosidic bond formation between the sugar moiety of an activated sugar donor molecule and a nucleophilic atom, for example, oxygen, nitrogen, sulphur or carbon of an acceptor molecule (Lairson et al (2008) Annu. Rev. Biochem., 77, 521-555). Donor sugar moieties are usually activated with a substituted phosphate leaving group. Most commonly these leaving groups are nucleoside diphosphates (e.g. UDP, GDP) and sometimes they are nucleoside monophosphates (e.g. CMP), lipid phosphates (e.g. dolichol phosphate) or phosphate. Glucosyl transferases are frequently involved in xenobiotic metabolism in plants. Typically, when herbicides are metabolized and inactivated in tolerant plants, glucosyl transferases are involved but more usually in a secondary role. For example, O-glucosylation (catalyzed by a UDP-glucosyl transferase enzyme) often occurs as a secondary metabolic reaction following on from a primary oxygenase-catalyzed metabolic reaction (typically catalyzed by a Cytochrome P450 enzyme) that results in hydroxylation of the herbicide (Lamoureux et al (1991) in Herbicide Resistance in Weeds and Crops (J. C. Caseley, G. W. Cussans, R. K. Atkin ed. pp 227-262, Butterworth Heinemann). Nevertheless, some herbicides are subject to direct glucosylation in some plants. For example, Metribuzin, a PSII acting amine herbicide is metabolized by direct N-glucosylation in tomatoes (Davis et al (1991) Plant Sci., 74, 73-80)) and direct N-glucosylation is also one of a number of mechanisms of metribuzin metabolism observed in soybean (Frear et al. (1985) Pest Biochem. Physiol., 23, 56-65). A number of herbicides representing different modes of action have structures with nucleophilic atoms in positions that could or do make them acceptor substrates for glucosyl transferases. Such herbicides include, for example, not only metribuzin but also pyridafol, amicarbazone, bentazon, chloridazone, amitrole, metamitron, indaziflam, triaziflam, flupoxam, aminopyralid, fluroxypyr, asulam, aclonifen, bromoxynil, halauxifen, rinskor, ioxynil, dinitramine, pendimethalin, chloramben, pyrimisulfan, chlorflurenol and picloram. Picloram for example is N-glucosylated at a low rate by a UDP glucosyl transferase from Arabidopsis (Loutre et al (2003) The Plant Journal, 34, 485-493). However, while observed as a naturally occurring route of metabolism, it has not, in the past, been clear to what (if any) extent direct glucosylation of herbicides has been quantitatively or, indeed, at all (given the lability of some glucosides) responsible for conferring tolerance to herbicides and neither, hitherto, has the route been exploited either as a transgenic or directed mutagenesis (genome editing) route to providing herbicide-resistance in crops.

The use of herbicide tolerance transgenes to engineer crops to become herbicide-tolerant and thereby to extend the use of certain herbicides to further crops is now a well-established technology. Herbicide-tolerance conferring transgenes generally encode either an altered and thereby herbicide-insensitive target site (e.g. a glyphosate insensitive 5-enolpyruvyl shikimate-3-phosphate synthase in the case of glyphosate tolerance; Funk et al (2006) PNAS, 103, 13010-13015; WO 1992004449) or an enzyme that metabolizes the herbicide to an inactive form (e.g. phosphinothricin N-acetyl transferase as in the case of glufosinate tolerance; DeBlock et al (1987) EMBO J., 6, 2513-2518; U.S. Pat. No. 5,276,268). Similarly, in situ mutagenesis (directed or otherwise) has been used to mutate, for example, acetolactate synthase (ALS) or Acetyl CoA carboxylase (ACCase) herbicide target genes in order to create mutant herbicide-tolerant crop lines (Rizwan et al (2015) Adv. life sci., vol. 3, pp. 01-08). Aside from the early examples of tolerance to the non-selective herbicides , glyphosate and glufosinate , there is now an extensive art around transgenes and methods to confer herbicide tolerance to herbicides which, for example, act by inhibiting 4-hydroxyphenylpyruvate synthase (e.g. WO 02/46387; WO2015135881; WO2010/085705), protoporphyrinogen oxidase (e.g. WO15092706; WO2013/189984) and also to several auxin type herbicides, notably dicamba (e.g. U.S. Pat. No. 7,022,896; U.S. Pat. No. 7,884,262; D'Ordine et al (2009) J. Mol. Biol., 392, 481-497) and 2,4 D (e.g. WO2005/107437), which act as agonists at auxin receptors.

PSII is a particularly important site of herbicide action but one that is relatively under-represented in terms of the availability of commercial herbicide-resistant transgenic crops. There are many classes and examples of commercialized PSII− herbicides and all of these act by binding to the D1 protein of the photosystem II complex and thereby blocking electron transport to plastoquinone (Mets and Thiel (1989) in Target Sites of Herbicide Action (CRC press Boger and Sandmann ed.), pp 1-24). For example, metribuzin is an amine PSII herbicide and bromoxynil is an example of an alcohol PSII herbicide. A nitrilase transgene that confers resistance to bromoxynil (Stalker et al (1988) Science, 242(4877):419-23) was commercialized in the past to enable bromoxynil use in cotton. Although certain PSII herbicides are naturally selective in certain crops (e.g. bromoxynil in wheat and atrazine in corn) crop safety is usually (apart from in the case of atrazine) quite limited in terms of application rate and, does not extend to high enough rates to provide broad spectrum weed control when applied over crops. In general, growers lack options to enable the use of the more potent and broad spectrum types of PSII herbicides at flexible timings and in a broad range of crops. Furthermore, it would be especially desirable to enable the use of PSII herbicides across a wider range of crops and particularly in combination with HPPD mode of action herbicides since this combination can provide synergistic and highly effective weed control (e.g. Walsh et al (2012) Weed Technol. 26, 341-347; Hugie et al (2008) Weed Science, 56, 265-270). Furthermore the combined use of PSII and HPPD herbicides also provides a valuable mixture option to help combat the increasing problem of herbicide-resistant weeds. Particularly effective modern broad spectrum classes of PSII herbicides are the alcohols and aminals of the types described for example in patents and patent applications CH633678, EP0297378, EP0286816, GB2119252, EP0334133, U.S. Pat. No. 4,600,430, U.S. Pat. No. 4,911,749, U.S. Pat. No. 4,857,099, U.S. Pat. No. 4,426,527, U.S. Pat. No. 4,012,223, WO2015018433, WO16162265, WO16156241, WO16128266, WO16071359, WO16071360, WO16071362, WO16071363, WO16071364, WO16071361, WO15193202, US2016318906, US2016262395, US2016251332, US2016264547, US2016200708, US2016159767, US2016159819, US2016159781, US2016168126, US2016066574 and US3932438 and, as for example, in structure I and structure II depicted below.

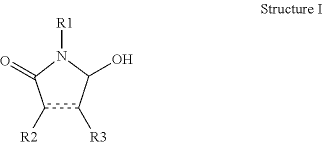

wherein R2 is halogen or C1-C3 alkoxy
and R3 is C1-C6 alkyl or C1-C3 alkoxy
and wherein R1 includes aromatic heterocycles (and partially unsaturated heterocycles), containing 1-3 nitrogens and further substituted at 1-3 positions on the ring with a broad range of substituents (H, C—C4 alkyl, t-Bu, halogen, CF3, SF5 etc.) as defined in the patent applications listed infra. Examples of aromatic headgroups R1 include substituted pyridazines, pyridines, pyrimidines, oxadiazoles, isoazoles and thiadiazoles.

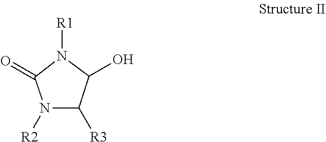

wherein R2 is C1-C6 alkyl, alkenyl, allyl, alkynyl or haloalkyl
and R3 is C1-C6 alkyl, alkoxy or allyl or hydrogen.
and wherein R1 includes aromatic heterocycles (and partially unsaturated heterocycles), containing 1-3 nitrogens and optionally substituted at 1-3 positions on the ring with a broad range of substituents (H, C alkyl, t-Bu, halogen, CF3, SF5 etc.) as defined in the patent applications listed infra. Examples of aromatic headgroups R1 include pyridazines, pyridines, pyrimidines, oxadiazoles, isoazoles and thiadiazoles
Some specific examples of these alcohol and aminal herbicide chemistries are depicted below as structures III to XII.

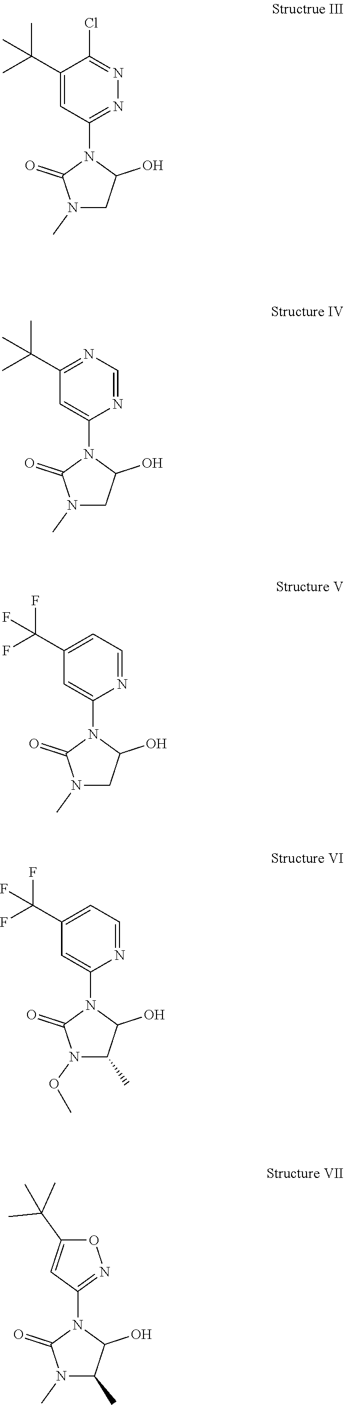 [SMILES: CN1CC(O)N(c2cc(C(C)(C)C)c(Cl)nn2)C1=O ; CN1CC(O)N(c2cc(C(C)(C)C)ncn2)C1=O ; CN1CC(O)N(c2cc(C(F)(F)F)ccn2)C1=O ; CON1C(=O)N(c2cc(C(F)(F)F)ccn2)C(O)[C@@H]1C ; C[C@@H]1C(O)N(c2cc(C(C)(C)C)on2)C(=O)N1C]

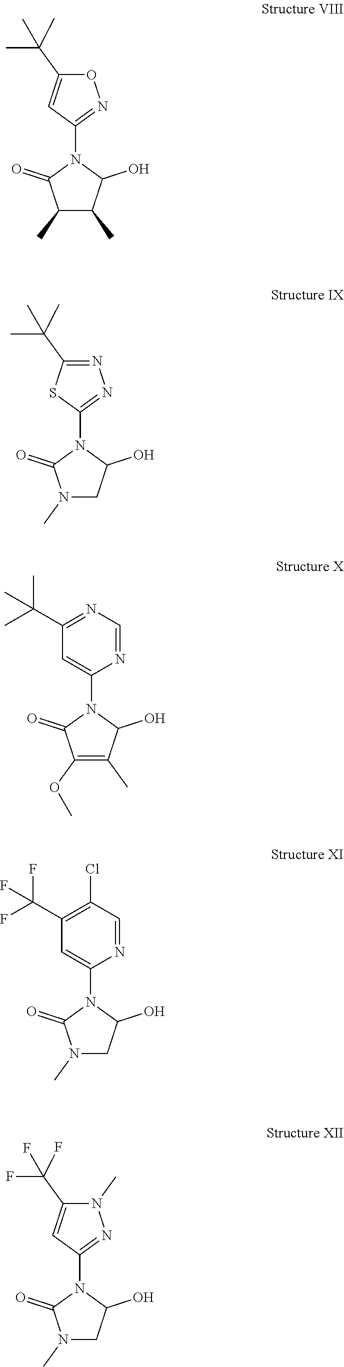 [SMILES: C[C@@H]1C(O)N(c2cc(C(C)(C)C)on2)C(=O)[C@@H]1C ; CN1CC(O)N(c2nnc(C(C)(C)C)s2)C1=O ; COC1=C(C)C(O)N(c2cc(C(C)(C)C)ncn2)C1=O ; CN1CC(O)N(c2cc(C(F)(F)F)c(Cl)cn2)C1=O ; CN1CC(O)N(c2cc(C(F)(F)F)n(C)n2)C1=O]

Accordingly, new methods and compositions for conferring herbicide tolerance to herbicides and, in particular, to amine, alcohol and aminal herbicides upon various crops and crop varieties are needed.

### BRIEF SUMMARY OF THE INVENTION

Compositions and methods for conferring herbicide resistance or tolerance upon plants towards certain classes of herbicide are provided. In particular these are amine, alcohol and aminal herbicides. The compositions include nucleotide and amino acid sequences for wild-type and mutant glucosyl transferase polypeptides. The polypeptides of the invention are mutant or wild type glucosyl transferases that are capable of catalyzing the transfer of glucose to certain herbicidal structures and that, thereby, confer resistance or tolerance in plants to amine, alcohol and aminal PSII herbicides. Particularly, polypeptides of the invention include mutant or wild-type bx-type UDP glucosyl transferases.

In one embodiment, the composition of the invention comprises a bx-type UDP glucosyl transferase polypeptide having at least 60%, 65%, 70%, 75%, 80%, 85%, 90%, 91%, 92%, 93%, 94%, 95%, 96%, 97%, 98% or 99% sequence identity to a sequence selected from the group consisting of : SEQ ID NO:1 (Zea mays bx9 sequence), SEQ ID NO:2 (Zea mays bx8 sequence), SEQ ID NO:3 (an Echinocloa bx sequence), SEQ ID NO:4 (a wheat bx sequence), SEQ ID NO:5 (a sorghum bx sequence), SEQ ID NO:6 (a barley bx sequence), SEQ ID NO:7 (an Alopecurus bx sequence) SEQ ID NO:8 (an Avena bx sequence) SEQ ID NO:9 (a rice bx sequence), SEQ ID NO:10 (a Larkspur bx sequence), SEQ ID NO: 11 (a rye bx sequence), SEQ ID NO:12 (a Brachypodium bx sequence), SEQ ID NO:13 (an Eleusine bx sequence), SEQ ID NO: 14 (a Setaria bx sequence) and SEQ ID NO:15 (a Dicanthelium bx sequence).

The compositions and processes of the invention are useful in methods directed to conferring resistance or tolerance to plants to certain herbicides. In particular embodiments, the methods comprise introducing into a plant at least one expression cassette comprising a promoter operably linked to a nucleotide sequence that encodes a bx-type UDP glucosyl transferase enzyme. The invention also includes the transgenic herbicide tolerant plants, varieties and their seeds and progeny comprising nucleic acid sequences that encode the polypeptides of the current invention that are the product of application of the above methods of the invention.

Methods of the present invention also comprise selectively controlling weeds in a field at a crop locus. In one embodiment, such methods involve over-the-top pre-or post-emergence application of a weed-controlling amount of an herbicide in a field at a crop locus that contains plants expressing a mutant endogenous or a heterologous bx-type UDP glucosyl transferase enzyme.

In a method for the control of unwanted vegetation, an herbicide is applied to the locus of a crop plant that expresses a bx-type UDP glucosyl transferase that is cognate for the said herbicide. The said herbicide is thereby converted to a herbicidally inactive glucoside which process of conversion leads to the crop expressing resistance or tolerance to the said herbicide and sequestering herbicide as the said glucoside into plant cell vacuoles.

In a further particular embodiment the herbicide is an amine, alcohol or aminal type PSII herbicide. In a yet further embodiment the herbicide is selected from the group consisting of structures: III, IV, V, VI, VII, VIII, IX, X, XI, XII, XIII, XIV, XV, XVI, XVII, XVIII, XIX, XX, XXI, XXII, XXIII, XXIV, XXV, XXVI, metribuzin, pyridafol, amicarbazone, bentazon, chloridazone, amitrole, metamitron, indaziflam, triaziflam, flupoxam, aminopyralid, fluroxypyr, asulam, aclonifen, bromoxynil, halauxifen, rinskor, ioxynil, dinitramine, pendimethalin, chloramben, pyrimisulfan, chlorflurenol and picloram.

In a further embodiment, the above described compositions, processes and methods of the invention comprise or utilize a wild-type bx-type UDP glucosyl transferase peptide.

In a further embodiment, the above described compositions, processes and methods of the invention comprise or utilize a mutant bx-type UDP glucosyl transferase peptide comprising one or more amino acid motifs selected from the group consisting of:
i. P(L,M,I,F)(P,A)X(Q,L,P,H)GH (SEQ ID NO: 60), wherein X=Y
ii. PFPX(Q,L)GH (SEQ ID NO: 61), wherein X=Y
iii. PFPXQGH (SEQ ID NO: 62), wherein X=Y
iv. P(L,M,I,F)(P,A)(F,Y)XGH (SEQ ID NO: 63), wherein X=any but preferably H,I,P,C,M
v. PFPFXGH (SEQ ID NO: 64), wherein X=any but preferably H,I,P,C,M
vi. S(E,D,K,G)DXA (SEQ ID NO: 65), wherein X=any but preferably F,Y
vii. ASEDXA (SEQ ID NO: 66), wherein X=any but preferably F,Y
viii. S(E,D,K,G)D(I,A)X (SEQ ID NO: 67), wherein X=any but preferably G,M,E,H,L,F,S,N,Q
ix. ASEDIX (SEQ ID NO: 68), wherein X=any but preferably G,M,E,H,L,F,S,N,Q
x. (L,M,V,I)X(A,D,R,V,E,K,G)(S,A,T,N)(S,C,A,F,M)(D,E,A)(S,A,E,G) (SEQ ID NO: 69), wherein X=any but preferably D
xi. (L,M)X(A,D)(S,A)(S,C,A)(D,E)A (SEQ ID NO: 70), wherein X=any but preferably D
xii. LXA(S,A)C(D,E)A (SEQ ID NO: 71), wherein X=any but preferably D
xiii. (C,F,V)(L,I,V)(F,L,I,V)(A,S,T,I,V,F)D(A,T,G,V,S)X(W,L) (SEQ ID NO: 72), wherein X=any but preferably T,C,I,V,G
xiv. CV(F,L,I)TDVXW (SEQ ID NO: 73), wherein X=any but preferably T,C,I,V,G
xv. (P,R,K,A)(S,L,T,V,A)(L,M)(G,P,L,V)(M,V,I,L)X(L,P,T)(S,N,T,A)SAA (SEQ ID NO:74), wherein X=any but preferably S,T,C,H,A,I,L,V
xvi. PALG(M,V,I)XTASAA (SEQ ID NO:75), wherein X=any but preferably S,T,C,H,A,I,L,V
xvii. (P,R,K,A)(S,L,T,V,A)(L,M)(G,P,L,V)(M,V,I,L)(F,R,M)(L,P,T)XSAA (SEQ ID NO:76), wherein X=any but preferably S
xviii. PALG(M,V,I)MTXSAA (SEQ ID NO:77), wherein X=any but preferably S
xix. (A,V,E)(F,T,Y)(R,Q,P)(A,R,M,S,L,T)LX(D,E,A,Q,R,K)(N,R,Q,A,K)(G,A,C) (SEQ ID NO: 78), wherein X=any but preferably T,Q,K,R,V,L,F,H
xx. AY(R,Q)TLXDK(G,A) (SEQ ID NO: 79), wherein X=any but preferably T,Q,K,R,V,L,F,H
xxi. (A,E,L)(E,D,L)(F,Y)AXLL (SEQ ID NO: 80), wherein X=any but preferably T,C,N,A,D,G,Q,V,I
xxii. E(E,D)FAXLL (SEQ ID NO: 81), wherein X=any but preferably T,C,N,A,D,G,Q,V,I
xxiii. (M,I,L)(G,E)(T,A,G,D,Q,R,P)(D,G,A,S,T,V,N)X(I,V,L)(A,G,E,D,Q,N,R,C)(Q,R,E,N,K, D)(I,L) (SEQ ID NO: 82), wherein X=any but preferably P,F,R,W,Y,H,K,L,M,E,I,S,N,G,C
xxiv. IE(T,A)(D,G,A)XL(A,G,E)(Q,R,E)I (SEQ ID NO: 83), wherein X=any but preferably P,F,R,W,Y,H,K,L,M,E,I,S,N,G,C
xxv. IE(T,A)(D,G)XL(A,G)EI (SEQ ID NO: 84), wherein X=any but preferably P,F,R,W,Y,H,K,L,M,E,I,S,N,G,C
xxvi. V(L,I)(Y,F)(I,A,V)S(L,I,F)G(T,S)X(A,V)(S,N,T,G,A) (SEQ ID NO: 85), wherein X=any but preferably V,W,F,I
xxvii. VLYVSFGSXAA (SEQ ID NO: 86), wherein X=any but preferably V,W,F,I
xxviii. V(L,I)(Y,F)(I,A,V)S(L,I,F)G(T,S)(M,L,I,V)(A,V)X (SEQ ID NO: 87), wherein X=any but preferably Q,K,R,L,V,M,C,T,S
xxix. VLYVSFGSMAX (SEQ ID NO: 88), wherein X=any but preferably Q,K,R,L,V,M,C,T,S
xxx. (V,I)(V,I)XWAPQ(E,Q,D)(E,K,D)(V,A)L (SEQ ID NO: 89), wherein X=any but preferably R,K
xxxi. (V,I)VXWAPQEEVL (SEQ ID NO: 90), wherein X=any but preferably R,K
xxxii. GWNS(A,M,T)(V,I,M,L,T,A)E(A,S,G)X(S,A,L,C,G)(E,Q,R,G,A,D)(T,G)(V,H,L)P (SEQ ID NO: 91), wherein X=any but preferably S,M,Q,W,T,F,A,V,L
xxxiii. TVEAX(S,A)EGV (SEQ ID NO: 92), wherein X=any but preferably S,M,Q,W,T,F,A,V,L
xxxiv. (E,Q,R,G,A,D)(T,G)(V,H,L)P(M,V)X(C,A,S) (SEQ ID NO: 93), wherein X=any but preferably G,S,T,A,F,Y,N,I,A
xxxv. EGVPMXC (SEQ ID NO: 94), wherein X=any but preferably G,S,T,A,F,Y,N,I,A
xxxvi. (C,S)(C,H,R,L,K)P(R,L,F,C,S,Y,H,Q)(H,G,F,S)XDQ (SEQ ID NO: 95), wherein X=any but preferably L
xxxvii. C(C,H)P(R,L)HXDQ (SEQ ID NO: 96), wherein X=any but preferably L
xxxviii. K(I,M)AX(A,D,E)(K,D)G (SEQ ID NO: 97), wherein X=any but preferably L,V,H,Q,P,T,F,Y,D,E,R,K,N
xxxix. KIAX(A,D)KG (SEQ ID NO: 98), wherein X=any but preferably L,V,H,Q,P,T,F,Y,D,E,R,K,N
xl. (R,K,G)(A,M,I,V,S)(E,K,M,L,I,R,G,S,N,H)(E,N,G,D,A,H,V,K,S,Q,I)(L,F,M)(K,G,R,Q, E,M)(S,D,E,Q,G,K,L,N,H,I,M)(R,A,K,V,E,M,I,Q,S)(A,V,S,M)(A,D,E,G,T,S,V,K,E,L,I, Y,R,N)(K,R,L,V,F,Q,S,D,E,A)(G,C,S,A,T)(I,T,A,L,V,F,M,S) (SEQ ID NO: 99), immediately upstream of and adjacently linked to a following peptide that either consists of or comprises at its N terminus a sequence selected from the group of GIGVD (SEQ ID NO: 102), GIGVDV (SEQ ID NO: 103), GIGVDVD (SEQ ID NO: 104), or GIGVDVDE (SEQ ID NO: 105)
xli. R(A,M)(K,M,L,I,R,G,S,N,H)(E,N,G,D,A,H,I)(L,F,M)(K,G,R,Q)(S,D,E,Q,G,K,L,N,H,I, M)(R,A,K,V,E,M,I,S)(A,V,S,M)(A,D,E,G,T,S,V,K,E,L,I)(K,R,Q,S,D,E,A)(G,C,S,A,T)(I, T,A,L,V,M,S) (SEQ ID NO: 100) immediately upstream of and adjacently linked to a following peptide consisting of or comprising at its N terminus a sequence selected from the group of GIGVD (SEQ ID NO: 102), GIGVDV (SEQ ID NO: 103), GIGVDVD (SEQ ID NO: 104), or GIGVDVDE (SEQ ID NO: 105) or any conservative variant of these sequences.
xlii. R(A,M)(K,M,L,I,G,N,H)(E,N,G,D,A,H)(L,M)(K,G,R,Q)(S,D,E,Q,G,K,L,N,H,I,M)(R,A, K,V,E,M,I)(A,V)(A,D,E,G,S,V,L)(K,R,Q,D,E)(G,C,S,A)(I,T,A,V) (SEQ ID NO: 101) immediately upstream of and adjacently linked to a following peptide consisting of or comprising at its N terminus a sequence selected from the group of GIGVD (SEQ ID NO: 102), GIGVDV (SEQ ID NO: 103), GIGVDVD (SEQ ID NO: 104), or GIGVDVDE (SEQ ID NO: 105) or any conservative variant of these sequences.

In a further embodiment, the above described compositions, processes and methods of the invention comprise or utilize a mutant bx-type UDP glucosyl transferase peptide comprising one or more amino acid residues at the amino acid position corresponding to the identified position relative to SEQ ID NO: 1, selected from the group consisting of:
a. Position 19—M
b. Position 21—Y
c. Position 22—any, preferably H,I,P,C or M
d. Position 78—any, preferably F or Y
e. Position 79—any, preferably G,M,E,H,L,F,S,N or Q
f. Position 86—any, preferably D
g. Position 117—any, preferably T,C,I,V or G
h. Position 135—any, preferably S,T,C,H,A,I,L or V
i. Position 138—any, preferably S
j. Position 143—any, preferably Y,F or W
k. Position 153—any, preferably T,Q,K,R,V, L, H or F
l. Position 194—any, preferably V,I,T,C,N,A,D,G or Q
m. Position 220—any, preferably P,F,R,W,Y,H,K,L,M,E,I,S,N,G or C
n. Position 279—any, preferably I,V,W or F
o. Position 281—any, preferably Q,K,R,L,V,M,C,T or S
p. Position 334—any, preferably R or K
q. Position 363—any, preferably S,M,Q,W,T,F,A,V or L
r. Position 370—any, preferably G,S,T,A,F,Y,N,I,A
s. Position 372—any, preferably E or Q
t. Position 376—any, preferably L
u. Position 432—any, preferably L,V,H,Q,P,T,F,Y,D,E,R,K,N
v. Position 437—a short peptide consisting of or comprising a sequence selected from the group of GIGVD (SEQ ID NO: 102), GIGVDV (SEQ ID NO: 103), GIGVDVD (SEQ ID NO: 104), or GIGVDVDE (SEQ ID NO: 105) or any conservative variant of these sequences.

It is clear from the above described mutant positions relative to SEQ ID NO: 1 and the above described mutant motifs that in some cases, the mutant position is found in multiple motifs. When this occurs, the skilled person will understand that the mutants can be stacked together, and that it is often desirable to do so. For example, the mutant positions 21 and 22 described above are both found in SEQ ID NOS: 61-64. SEQ ID NOs: 61 and 62 are directed to the motif surrounding position 21 and SEQ ID NOs: 63-64 are directed to the motif surrounding position 22.

Further methods of the invention also include the use of mutagenesis and recombination (for example directed using chimeric oligonucleotides, Meganucleases, Zinc Fingers, TALEN or CRISPR) to introduce specific strand breaks, recombinational insertions and mutations so as to engineer in situ changes in plant genomes so that the thus mutated plant genome is then altered so that it is able to express one or more of the mutant bx-type UDP glucosyl transferase polypeptides of the current invention and is thus made herbicide-tolerant. Thus the invention also includes mutated herbicide tolerant plants, varieties and their seed and progeny that are derived from the product of application of the above methods of the invention.

Exemplary mutant bx-type UDP glucosyl transferase polypeptides according to the invention correspond to the amino acid sequences set forth in SEQ ID NOS: 16-59, and variants thereof. Nucleic acid molecules comprising polynucleotide sequences that encode the wild type and mutant glucosyl transferase polypeptides of the invention are inherent in the disclosure of the polypeptide sequences. Compositions also include expression cassettes comprising a promoter operably linked to a nucleotide sequence that encodes a polypeptide of the invention, alone or in combination with one or more additional nucleic acid molecules encoding polypeptides that confer desirable traits. Transformed plants, plant cells, and seeds comprising an expression cassette of the invention are further provided.

In other embodiments, methods are also provided for the assay, characterization, identification, and selection of the herbicide-active glucosyl transferases of the current invention.

### BRIEF DESCRIPTION OF THE DRAWINGS

FIG. 1—Alignment of wild type bx glucosyl transferase amino acid sequences SEQ ID NO 1-10.

FIG. 2—depicts a UDP/luminescence standard curve

FIG. 3—Km and kcat estimations (see Table 11) for DIMBOA and herbicides V, VI and IX in respect of C-terminally his-tagged SEQ ID NO: 1

FIG. 4—Km and kcat determinations for certain C-terminally his tagged mutants of Zea mays bx9 glucosyl transferase in respect of herbicide VI

FIG. 5—Example of a binary vector used to transform tobacco to express the glucosyl transferases corresponding to SEQ ID NO: 1

FIG. 6—Transgenic and wild type tobacco plants14 DAT after treatment with herbicide V and VI.

FIG. 7A-7B—O-glucosides of structures V and VI

FIG. 8A-8H—Examples of LC/MS chromatograms and spectra of herbicide glucosides

FIG. 9 Km and kcat determinations for the C-terminally his tagged Zea mays bx9 glucosyl transferase SEQ ID NO:1 having three mutations M279F, H375Y and E339A and with metribuzin as acceptor substrate.

FIG. 10 Schematic drawing of CRISPR-Cas9 vector 23935 expressing sgRNAs with targeting sequence xZmBx9V1, xZmBx9V2, xZmBx9V3, and xZmBx9V4

FIG. 11 Schematic drawing of targeted gene replacement donor vector 23939 with homology sequences xJHAXBx9-01 and xJHAXBx9-02 flanking the desired DNA fragment xB73Bx9-01

FIG. 12 Schematic drawing of CRISPR-Cas9 vector 23935 and donor 23939 combinations for biolistic co-delivery .Green bar represent 6 amino acids change from the wilde type genomic sequence.

FIG. 13 Schematic drawing of targeted gene replacement donor vector 23984 with homology sequences xJHAXBx9 and cZmUGTBx9 flanking the desired DNA fragment

FIG. 14 Schematic drawing of CRISPR-Cas9 vector 23792 expressing sgRNAs with targeting sequence xZmBx9-M279F

FIG. 15 Schematic drawing of CRISPR-Cas9 vector 24001 expressing sgRNAs with targeting sequence xZmBx9-M279F

FIG. 16 Schematic drawing of CRISPR-Cas9 vector 23792 or 24001 and donor 23984 combinations for biolistic co-delivery. Green bars represent 6 amino acids change from the wilde type genomic sequence.

FIG. 17 Schematic drawing of CRISPR-Cas9 vector 24096 expressing gRNAs with targeting sequence xZmBx9 Target3r

FIG. 18 Schematic drawing of CRISPR-Cas9 vector 24098 expressing gRNAs with targeting sequence xZmBx9Target4r

FIG. 19 Schematic drawing of CRISPR-Cas9 vector 24099 expressing gRNAs with targeting sequence xZmBx9Target7

FIG. 20 Schematic drawing of CRISPR-Cas9 vector 24100 expressing gRNA with targeting sequence xZmBx9Target2

FIG. 21 Schematic drawing of targeted gene replacement donor vector 24101 with homology sequences xJHAXBx9-05 and xJHAXBx9-02 flanking the desired DNA fragment xZmUGTBx9-17

FIG. 22 Schematic drawing of CRISPR-Cpf1 vector and donor combinations for biolistic co-delivery. Green bars represent 6 amino acids change from the wild type genomic sequence.

### LISTING OF THE TABLES

Table 1 Mutations in SEQ ID NO: 1 (maize bx9) useful for providing enhanced glucosyl transferase activity to herbicides
Table 2 Mutations in SEQ ID NO: 2 (maize bx8) useful for providing enhanced glucosyl transferase activity to herbicides
Table 3 Mutations in SEQ ID NO: 3 (Echinocloa bx) useful for providing enhanced glucosyl transferase activity to herbicides
Table 4 Mutations in SEQ ID NO: 4 (wheat bx) useful for providing enhanced glucosyl transferase activity to herbicides
Table 5 Mutations in SEQ ID NO: 5 (sorghum bx) useful for providing enhanced glucosyl transferase activity to herbicides
Table 6 Mutations in SEQ ID NO: 6 (barley bx) useful for providing enhanced glucosyl transferase activity to herbicides
Table 7 Mutations in SEQ ID NO: 7 (alopecurus bx) useful for providing enhanced glucosyl transferase activity to herbicides
Table 8 Mutations in SEQ ID NO: 8 (avena bx) useful for providing enhanced glucosyl transferase activity to herbicides
Table 9 Mutations in SEQ ID NO: 9 (rice bx) useful for providing enhanced glucosyl transferase activity to herbicides
Table 10. Estimates of kinetic parameters for Zea mays bx9 (C-terminally his tagged SEQ ID NO: 1) assayed with DIMBOA and herbicides V, VI and IX as acceptor substrates
Table 11 Preferred and most preferred amino acid substitutions at a range of positions within the polypeptide sequence of SEQ ID NO: 1.
Table 12 Estimated kinetic parameters of the w/t and of various mutants of Zea mays bx9 glucosyl transferase assayed versus a range of herbicides
Table 13 Activities with various alcohol and aminal herbicides tested as substrates of w/t and mutant forms of Zea mays bx9 glucosyl transferase.
Table 14 Activities with various alcohol and aminal herbicides tested as substrates of w/t bx glucosyl transferases from various species.
Table 15 Relative activities with various alcohol and aminal herbicides tested as substrates of w/t and mutant forms of various bx-type glucosyl transferases
Table 16a Luminescence assay results for mutants at positions 19, 117, 135, 279 and 334 of SEQ ID No: 1 assayed with 2 mM metribuzin
Table 16b Luminescence assay results for mutants at various positions of SEQ ID No: 1 assayed with 2 mM metribuzin
Table 17 Luminescence assay results for mutants at various positions of SEQ ID No: 17 assayed with 2 mM metribuzin
Table 18 GH evaluation of percent damage to w/t/ and transgenic tobacco plants expressing either SEQ ID No 1 or SEQ ID No 2 at 14 DAT with 30 g/ha of compound VI
Table 19 GH evaluation of percent damage to tobacco plant lines expressing mutant forms of Zea mays bx9 glucosyl transferase after treatment with different herbicides
Table 20 Targeted allele replacement with different donor size
Table 21 Targeted allele replacement efficiency comparison with single or double cleavage
Table 22 Comparison of targeted large gene replacement efficiency with Cpf1 and Cas9 system.

### DETAILED DESCRIPTION OF THE INVENTION

It is to be understood that this invention is not limited to the particular methodology, protocols, cell lines, plant species or genera, constructs, and reagents described herein as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a plant” is a reference to one or more plants and includes equivalents thereof known to those skilled in the art, and so forth. As used herein, the word “or” means any one member of a particular list and also includes any combination of members of that list (i.e., includes also “and”).

The present invention provides compositions and methods directed to conferring herbicide resistance or tolerance to plants. Compositions include amino acid sequences for polypeptides having herbicide glucosylating activity, variants and fragments thereof. Nucleic acids that encode the polypeptides of the invention are inherently disclosed. Methods for conferring herbicide resistance or tolerance to plants, particularly resistance or tolerance to certain classes of herbicides such as certain amine, alcohol and aminal PSII herbicides that are substrates for certain glucosyl transferases are further provided. Methods are also provided for selectively controlling weeds in a field at a crop locus and for the assay, characterization, identification and selection of the glucosyl transferase polypeptides that provide herbicide tolerance.

Methods are also provided for selectively controlling weeds in a field at a crop locus wherein the herbicides that are substrates for the glucosylating polypeptides of the invention are used alone or in combination with other herbicides and in particular in combination with HPPD herbicides.

Within the context of the present invention the terms photosystem II (PSII) herbicide and D1-protein binding herbicide are synonymous. “PSII herbicides” are herbicides whose primary site of action is PSII. They bind at the plastoquinone binding site of the D1 protein of the photosystem II complex and thereby block the flow of electrons to plastoquinone and thence to cytochrome b6f, PS1 and to NADP⁺. PSII herbicides prevent the conversion of absorbed light energy into electrochemical energy which results in the production of triplet chlorophyll and singlet oxygen which induce the peroxidation of membrane lipids. (E. Patrick Fuerst and Michael A. Norman, Weed Science (1991), Vol. 39, No. 3 pp. 458-464). Many PSII herbicide types are well known and described elsewhere herein and in the literature and, for example, current commercial types are listed in the HRAC “world of herbicides” chart at www.hracglobal.com. As used herein, the term “PSII herbicides” refers to herbicides where inhibition of electron transport from PSII is at least part of the herbicide's mode of action on plants.

Within the context of the present invention the terms hydroxy phenyl pyruvate dioxygenase (HPPD), 4-hydroxy phenyl pyruvate dioxygenase (4-HPPD) and p-hydroxy phenyl pyruvate dioxygenase (p-HPPD) are synonymous.

“HPPD herbicides” are herbicides that are bleachers and whose primary site of action is HPPD. Many are well known and described elsewhere herein and in the literature (Hawkes “Hydroxyphenylpyruvate Dioxygenase (HPPD)—The Herbicide Target.” In Modern Crop Protection Compounds. 2^{nd} Edition. Eds. Krämer, Schirmer, Jeschke and Witschel Eds., Germany: Wiley-VCH, 2012. Ch. 4.2, pp. 225-235; Edmunds and Morris “Hydroxyphenylpyruvate dioxygenase (HPPD) Inhibitors: Triketones.” In Modern Crop Protection Compounds. 2^{nd} Edition. Eds. Krämer, Schirmer, Jeschke and Witschel. Weinheim, Germany: Wiley-VCH, 2012. Ch. 4.3, pp. 235-262). As used herein, the term “HPPD herbicides” refers to herbicides that act either directly or indirectly to inhibit HPPD, where the herbicides are bleachers or where inhibition of HPPD is at least part of the herbicide's mode of action on plants.

As used herein, plants which are substantially “tolerant” to a herbicide exhibit, when treated with said herbicide, a dose/response curve which is shifted to the right when compared with that exhibited by similarly subjected non tolerant like plants. Such dose/response curves have “dose” plotted on the x-axis and “percentage kill or damage”, “herbicidal effect” etc. plotted on the y-axis. Tolerant plants will typically require at least twice as much herbicide as non-tolerant like plants in order to produce a given herbicidal effect. Plants which are substantially “resistant” to the herbicide exhibit few, if any, necrotic, lytic, chlorotic or other lesions or, at least, none that impact significantly on yield, when subjected to the herbicide at concentrations and rates which are typically employed by the agricultural community to kill weeds in the field.

As used herein, the term “confer” refers to providing a characteristic or trait, such as herbicide tolerance or resistance and/or other desirable traits to a plant.

As used herein, the term “heterologous” when used in reference to a gene or nucleic acid refers to a gene encoding a factor that is not in its natural environment (i.e., has been altered by the hand of man). For example, a heterologous gene may include a gene from one species introduced into another species. A heterologous gene may also include a gene native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the invention, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, in embodiments, a “heterologous” polynucleotide is a polynucleotide not naturally associated with a host cell into which it is introduced, including non-naturally occurring multiple copies of a naturally occurring polynucleotide. For example, in the present application a maize glucosyl transferase gene that was transgenically expressed back into a maize plant would still be described as “heterologous” DNA.

A variety of additional terms are defined or otherwise characterized herein.

### Glucosyl Transferase Sequences

The compositions of the invention include isolated or substantially purified glucosyl transferase polynucleotides and polypeptides as well as host cells comprising the polynucleotides.

The polypeptides of the invention are glucosyl transferases that are capable of catalyzing the transfer of glucose to certain herbicides and that, thereby, when expressed in plants, confer resistance or tolerance in plants to the said herbicides. Particularly, polypeptides of the invention include mutant or wild-type benzoxazinoid (bx)-type UDP glucosyl transferases.

Benzoxazinoids are protective secondary metabolites found in numerous species of the Poaceae family of monocotyledenous plants as well as in single species within some families of dicotyledenous plants. The pathway of benzoxazinoid biosynthesis in Poaceae is thought to be monophyletic whereas benzoxazinoid biosynthesis is thought to have evolved independently in dicots. The genes, enzymes and pathway of benzoxazinoid biosynthesis and, more particularly, the glucosyl transferases involved are described in some considerable detail in the literature (Frey et al. (2009) Phytochemistry 70, 1645-1651; Dutartre et al (2012) BMC Evol. Biol. 12, 64; Dick et al (2012) Plant Cell 24, 915-928; Makowska et al (2015) Acta. Physiol. Plant (2015) 37, 176).

In the current application polypeptide sequences are defined as being “bx-type UDP glucosyl transferases” if they are capable of catalyzing glucosylation of either or both of 2,4-dihydroxy-1,4-benzoxazin-3-one (DIBOA) and 2,4-dihydroxy-7-methoxy-1,4-benzoxazin-3-one (DIMBOA) and have amino acid sequences that comprise all three of the polypeptide sequences (V,L,I,A)(R,K,Q,G)D(L,M) (SEQ ID 106), (P,T)(F,L,M,A,I)(P,A)(F,Y,L,A) (Q,L,P)GH (SEQ ID 107) and A(W,R)(G,A,S)(L,I)A (SEQ ID 108). In addition mutants, homologues and paralogues of these sequences that, on the basis of sequence alignments, the skilled man would annotate as bx-type UDP-glucosyl transferases are also included in this definition.

It is to be understood that throughout the description of the invention herein that a wild-type or mutant bx-type UDP-glucosyl transferase is a glucosyl transferase and that statements made regarding either wild-type or mutant glucosyl transferases apply equally to bx-type UDP-glucosyl transferases. Similarly, wild-type and mutant glucosyl transferases and/or wild-type and mutant bx-type UDP-glucosyl transferases are interchangeable in the various embodiments described herein, such as their use in expression cassettes, in transgenic plants and the methods of the invention.

Mutant glucosyl transferase polypeptides of the current invention have amino acid changes at one or more positions relative to the starting wild type sequence from which they are derived, and exhibit an enhanced ability to confer tolerance to one or more amine, alcohol or aminal PSII herbicides. Mutant glucosyl transferase enzymes that confer enhanced tolerance to a given herbicide may, for example, do so by virtue of exhibiting, relative to the like unmutated starting enzyme, under normal physiological conditions of temperature, pH and concentrations of UDP glucose
a) a lower Km value for the herbicide;
b) a higher kcat value for converting the herbicide to a glucose conjugate of the herbicide;
c) a higher catalytic efficiency (i.e. a higher value of kcat/Km) for converting herbicide to a glucose conjugate of the herbicide.

Here physiological concentrations of UDP-glucose are taken to be in the range from about 0.1 to about 2 mM UDP glucose and, preferably, about 0.5 mM. Similarly, physiological conditions of pH are from 7 to 7.5 and of temperature from 10 to 35 C but, preferably, for standard comparative measurement are fixed here as about pH 7.5 and 25 C.

Exemplary mutations that provide improved kcat and kcat/Km values versus various herbicides within the context of glucosyl transferase polypeptides SEQ ID NO: 1-9 are listed in Tables 1-9. Nucleic acids that encode the bx-type UDP glucosyl transferase polypeptides of the invention and fragments thereof are implicit in the provided polypeptide sequences.

DNA sequences encoding improved mutated glucosyl transferases of the current invention are used in the provision of transgenic plants, crops, plant cells and seeds that offer enhanced tolerance or resistance to one or more herbicides, and especially to amine, alcohol and aminal PSII herbicides, as compared to like, non-transgenic, plants.

Knowledge of the DNA sequences that encode improved mutated glucosyl transferases of the current invention is also used in the directed design and provision, for example by targeted genome editing, of mutant plants, crops, plant cells and seeds that offer enhanced tolerance or resistance to one or more herbicides, and especially to certain PSII herbicides, as compared to like non-mutated plants.

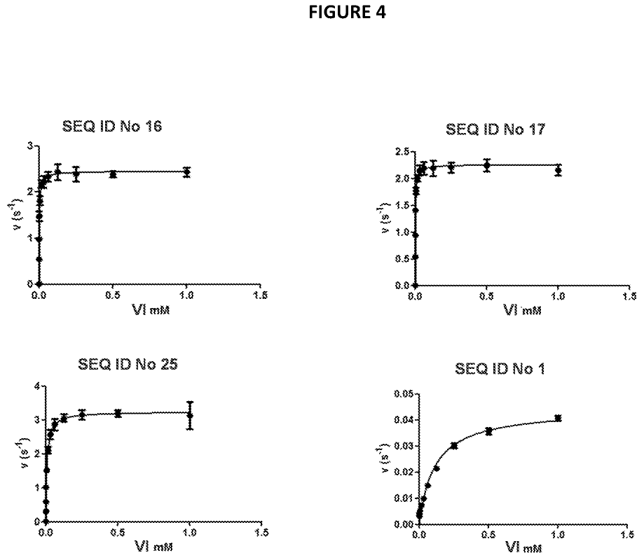

Increases in the value of kcat/Km in respect of an herbicide are of particular value in improving the ability of a glucosyl transferase to confer resistance to the said herbicide. So, for example, C terminally his tagged SEQ ID NO: 1 (Zea mays bx9 glucosyl transferase) which exhibits a relatively modest value of kcat/Km (Table 10) in respect of, for example, compound VI (in the range ˜0.3/mM/s) exhibits much increased values of kcat/Km when various mutations of the current invention are incorporated into the sequence (see for example Table 12 and FIG. 4). Accordingly transgenic (Table 16) expression of the polypeptide of SEQ ID No: 17 in tobacco confers a considerably higher level of resistance to compound VI than does like expression of SEQ ID NO 1.

Site-directed mutations of genes encoding plant-derived glucosyl transferases are selected so as to encode, for example, the amino acid changes listed in tables 1-9 and, for example, are as listed elsewhere herein and are applied either singly or in combination. Genes encoding such mutant forms of plant glucosyl transferases are useful for making crop plants resistant to herbicides that are substrates of these enzymes Plant glucosyl transferase genes so modified are especially suitable in the context of both in situ-mutated (genome-edited) and transgenic plants in order to confer herbicide tolerance or resistance upon crop plants.

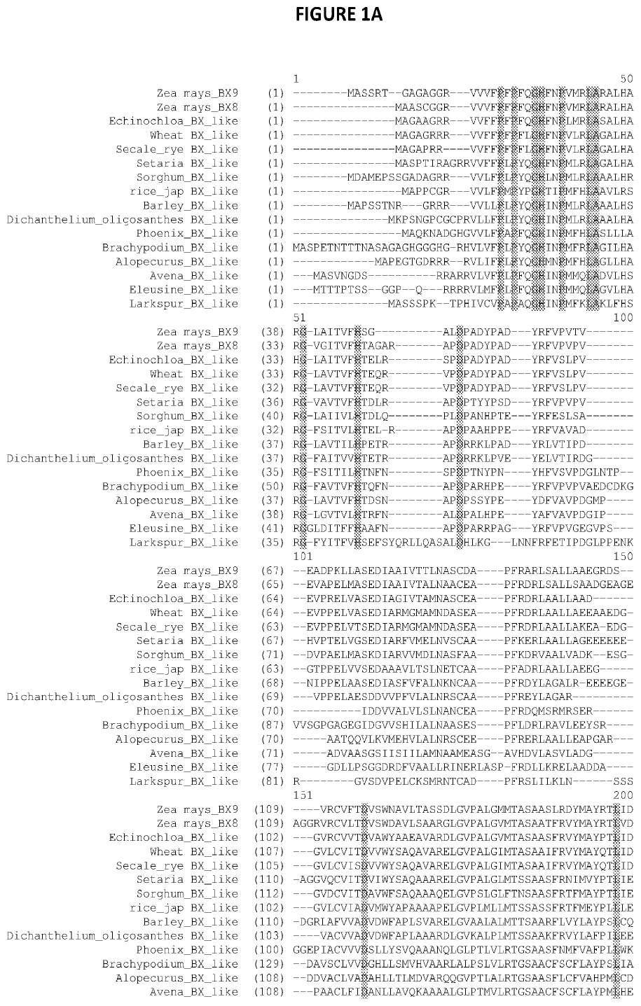

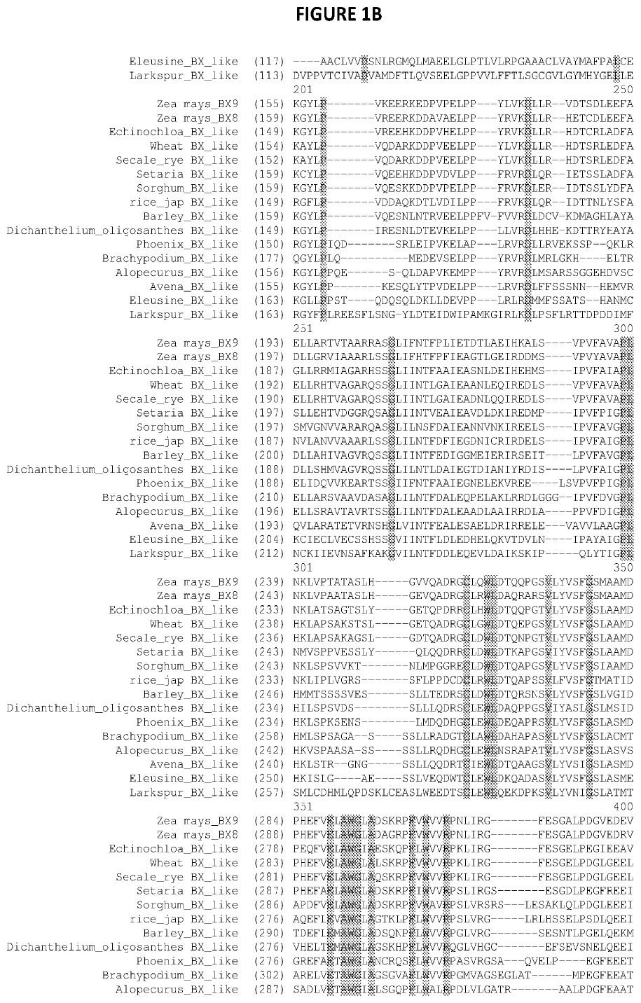

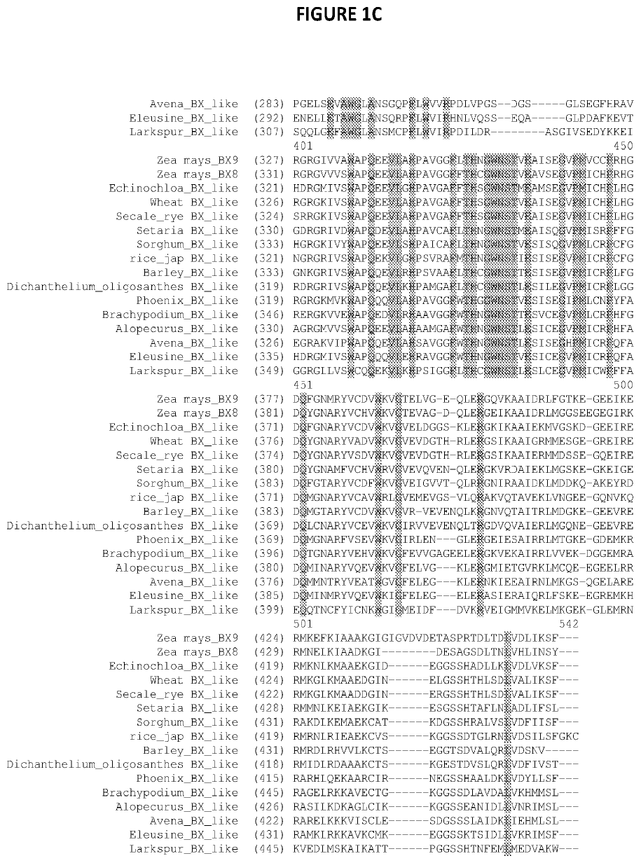

Many glucosyl transferase sequences are known in the art and can be used to generate mutant glucosyl transferase sequences by making the corresponding amino acid substitutions, deletions, and additions described herein. For example, a known or suspected glucosyl transferase reference sequence can be aligned with, for example, SEQ ID NO: 1-9 using standard sequence alignment tools (e.g. Align X using standard settings in Vector NTI and as depicted for example in FIG. 1) and the corresponding amino acid substitutions, deletions, and/or additions described herein with respect to, for example, SEQ ID NO: 1 can be made in the reference sequence.

In one embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:1 (the bx9 glucosyl transferase amino acid sequence of Zea mays) wherein the polypeptide contains one or more substitution(s), additions, or deletion(s) corresponding to the amino acid positions listed in column 1 of Table 1. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 1. In yet another embodiment, the polypeptide comprises one or more substitutions corresponding to a conservative variant of the amino acids listed in column 2 of Table 1. For example, the polypeptide may comprise a mutation corresponding to amino acid position 279 of SEQ ID NO: 1, wherein that amino acid is replaced with a phenylalanine or a conservative substitution of phenylalanine.

In a further embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:2 (the bx8 glucosyl transferase amino acid sequence of Zea mays) wherein the polypeptide contains one or more substitution(s), additions, or deletion(s) corresponding to the amino acid positions listed in column 1 of Table 2. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 2. In yet another embodiment, the polypeptide comprises one or more substitutions corresponding to a conservative variant of the amino acids listed in column 2 of Table 2. For example, the polypeptide may comprise a mutation corresponding to amino acid position 121 of SEQ ID NO: 2, wherein that amino acid is replaced with a valine or a conservative substitution of valine.

In a further embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:3 (the bx glucosyl transferase amino acid sequence of Echinocloa) wherein the polypeptide contains one or more substitution(s), additions, or deletion(s) corresponding to the amino acid positions listed in column 1 of Table 3. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 3. In yet another embodiment, the polypeptide comprises one or more substitutions corresponding to a conservative variant of the amino acids listed in column 2 of Table 3. For example, the polypeptide may comprise a mutation corresponding to amino acid position 273 of SEQ ID NO: 3, wherein that amino acid is replaced with a phenylalanine or a conservative substitution of phenylalanine.

In a further embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:4 (a bx glucosyl transferase amino acid sequence of wheat) wherein the polypeptide contains one or more substitution(s), additions, or deletion(s) corresponding to the amino acid positions listed in column 1 of Table 4. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 4. In yet another embodiment, the polypeptide comprises one or more substitutions corresponding to a conservative variant of the amino acids listed in column 2 of Table 4. For example, the polypeptide may comprise a mutation corresponding to amino acid position 278 of SEQ ID NO: 4, wherein that amino acid is replaced with a phenylalanine or a conservative substitution of phenylalanine.

In a further embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:5 (the bx glucosyl transferase amino acid sequence of Sorghum) wherein the polypeptide contains one or more substitution(s), additions, or deletion(s) corresponding to the amino acid positions listed in column 1 of Table 4. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 4. In yet another embodiment, the polypeptide comprises one or more substitutions corresponding to a conservative variant of the amino acids listed in column 2 of Table 4. For example, the polypeptide may comprise a mutation corresponding to amino acid position 281 of SEQ ID NO: 5, wherein that amino acid is replaced with a phenylalanine or a conservative substitution of phenylalanine.

In a further embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:6 (the bx glucosyl transferase amino acid sequence of barley) wherein the polypeptide contains one or more substitution(s), additions, or deletion(s) corresponding to the amino acid positions listed in column 1 of Table 6. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 6. In yet another embodiment, the polypeptide comprises one or more substitutions corresponding to a conservative variant of the amino acids listed in column 2 of Table 6. For example, the polypeptide may comprise a mutation corresponding to amino acid position 285 of SEQ ID NO: 6, wherein that amino acid is replaced with a phenylalanine or a conservative substitution of phenylalanine.

In a further embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:7 (the bx glucosyl transferase amino acid sequence of Alopecurus) wherein the polypeptide contains one or more substitution(s), additions, or deletion(s) corresponding to the amino acid positions listed in column 1 of Table 7. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 7. In yet another embodiment, the polypeptide comprises one or more substitutions corresponding to a conservative variant of the amino acids listed in column 2 of Table 7. For example, the polypeptide may comprise a mutation corresponding to amino acid position 282 of SEQ ID NO: 7, wherein that amino acid is replaced with a phenylalanine or a conservative substitution of phenylalanine.

In a further embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:8 (the bx glucosyl transferase amino acid sequence of Avena) wherein the polypeptide contains one or more substitution(s), additions, or deletion(s) corresponding to the amino acid positions listed in column 1 of Table 8. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 8. In yet another embodiment, the polypeptide comprises one or more substitutions corresponding to a conservative variant of the amino acids listed in column 2 of Table 8. For example, the polypeptide may comprise a mutation corresponding to amino acid position 278 of SEQ ID NO: 8, wherein that amino acid is replaced with a phenylalanine or a conservative substitution of phenylalanine.

In a further embodiment, the compositions of the invention comprise a mutant bx-type UDP-glucosyl transferase polypeptide having at least about 60%, 65%, 70%, 75%, 80%, 85%, 90%, 91%, 92%, 93%, 94%, 95%, 96%, 97%, 98%, 99% or more sequence identity to SEQ ID NO:9 (rice) wherein the polypeptide contains one or more substitution(s), additions, or deletion(s) corresponding to the amino acid positions listed in column 1 of Table 9. In various embodiments, an amino acid at one or more position(s) listed in column 1 is replaced with any other amino acid. In another embodiment, the polypeptide comprises one or more amino acid substitutions corresponding to the amino acid substitution(s) listed in column 2 of Table 9. In yet another embodiment, the polypeptide comprises one or more substitutions corresponding to a conservative variant of the amino acids listed in column 2 of Table 9. For example, the polypeptide may comprise a mutation corresponding to amino acid position 271 of SEQ ID NO:

9, wherein that amino acid is replaced with a phenylalanine or a conservative substitution of phenylalanine.

In particular embodiments, the amino acid sequence of the mutant bx-type glucosyl transferase polypeptides of the invention are selected from the group consisting of SEQ ID NO: 16-59.

TABLE 1
Exemplary glucosyl transferase mutations
in Zea maize bx9 (SEQ ID No 1)

| Mutable amino | |
|---|---|
| acid position | |
| relative to | |
| SEQ ID NO: 1 | Substitution or addition* |
| 19(F) | M |
| 21(F) | Y |
| 22(Q) | H, I, M, C, P |
| 76(E) | M, L, I |
| 78(I) | F, Y |
| 79(A) | G, E, M, F, L, H, Q, N, S |
| 81(I) | W, C, V |
| 82(V) | A, C, P |
| 86(N) | D |
| 116(V) | L, I |
| 117(S) | T, C, I, V, G |
| 118(W) | Y, F |
| 135(M) | H, S, T, I, L, A, C, V |
| 136(M) | P |
| 138(A) | S |
| 143(L) | M, Y, K, F, W |
| 153(I) | T, Q, K, R, V, L, F, H |
| 181(L) | C, I, M |
| 191(F) | M, T, I, L |
| 194(L) | T, C, N, A, G, Q, I, V, D |
| 195(L) | I |
| 198(T) | V |
| 199(V) | M, N, H, Y |
| 210(F) | M, W |
| 220(T) | P, F, W, Y, H, K, L, M, S, N, R, G, C, I, E |
| 279(M) | V, W, F, I |
| 280(A) | V |
| 281(A) | C, Q, K, R, L, M, V, T, S |
| 334(A) | R, K |
| 363(I) | S, Q, W, A, V, L, F, T, M |
| 370(V) | S, T, N, H, F, T, A, I, G, Y |
| 372(C) | I |
| 376(G) | L, C, M |
| 432(A) | L, V, H, Q, P, T, F, Y, D, E, R, K, N |

*Unless otherwise denoted, the amino acids and peptides listed in this column represent some potential substitutions at the indicated position.

TABLE 2
Exemplary glucosyl transferase mutations
in Zea maize bx8 (SEQ ID No 2)

| Mutable amino | |
|---|---|
| acid position | |
| relative to | |
| SEQ ID NO: 2 | Substitution or addition* |
| 14(F) | M |
| 16(F) | Y |
| 17(Q) | H, I, M, C, P |
| 74(E) | M, L, I |
| 76(I) | F, Y |
| 77(A) | G, E, M, F, L, H, Q, N, S |
| 79(I) | W, C, V |
| 80(V) | A, C, P |
| 84(N) | D |
| 120(V) | L, I |
| 121(S) | T, C, I, V, G |
| 122(W) | Y, F |
| 139(V) | H, S, T, I, L, A, C, M, V |
| 140(M) | P |
| 142(A) | S |
| 147(F) | M, Y, K, L, W, F |
| 157(V) | T, Q, K, R, I, L, F, H |
| 185(L) | C, I, M |
| 195(F) | M, T, I, L |
| 198(L) | T, C, N, A, G, Q, I, V, D |
| 199(L) | I |
| 202(V) | V, T |
| 203(I) | M, N, H, Y, V |
| 214(F) | M, W |
| 224(T) | P, F, W, Y, H, K, L, M, S, N, R, G, C, I, E |
| 283(M) | V, W, F, I |
| 284(A) | V |
| 285(A) | C, Q, K, R, L, M, V, T, S |
| 338(S) | R, K, A |
| 367(V) | S, I, Q, W, A, L, F, T, M |
| 374(I) | S, T, N, H, F, T, A, V, G, Y |
| 376(H) | I, C |
| 380(G) | L, C, M |
| 437(A) | L, V, H, Q, P, T, F, Y, D, E, R, K, N |
| 442(D) | peptides, GIGVD, GIGVDV, |
| | GIGVDVD or GIGVDVDE |

*Unless otherwise denoted, the amino acids and peptides listed in this column represent some potential substitutions at the indicated position.

TABLE 3
Exemplary glucosyl transferase mutations
in Echinocloa bx (SEQ ID No 3)

| Mutable amino | |
|---|---|
| acid position | |
| relative to | |
| SEQ ID NO: 3 | Substitution or addition* |
| 14(F) | M |
| 16(F) | Y |
| 17(Q) | H, I, M, C, P |
| 73(E) | M, L, I |
| 75(I) | F, Y |
| 76(A) | G, E, M, F, L, H, Q, N, S |
| 78(I) | W, C, V |
| 79(V) | A, C, P |
| 83(N) | D |
| 110(V) | L, I |
| 111(A) | T, C, I, V, S, G |
| 112(W) | Y, F |
| 129(V) | M, H, S, T, I, L, A, C |
| 130(M) | P |
| 132(A) | S |
| 137(F) | L, M, Y, K, W, F |
| 147(I) | T, Q, K, R, V, L, F, H |
| 175(L) | C, I, M |
| 185(F) | M, T, I, L |
| 188(L) | T, C, N, A, G, Q, I, V, D |
| 189(L) | I |
| 192(M) | T, V |
| 193(I) | V, M, N, H, Y |
| 204(I) | F, M, W |
| 214(N) | P, F, W, Y, H, K, L, M, S, T, R, G, C, I, E |
| 273(L) | V, W, F, I, M |
| 274(A) | V |
| 275(A) | C, Q, K, R, L, M, V, T, S |
| 328(S) | A, R, K |
| 357(M) | S, Q, W, A, V, L, F, T, I |
| 364(I) | S, T, N, H, F, T, A, V, G, Y |
| 366(H) | I, C |
| 370(G) | L, C, M |
| 427(A) | L, V, H, Q, P, T, F, Y, D, E, R, K, N |
| 432(D) | peptides, GIGVD, GIGVDV, GIGVDVD |
| | or GIGVDVDE |

*Unless otherwise denoted, the amino acids and peptides listed in this column represent some potential substitutions at the indicated position.

TABLE 4
Exemplary glucosyl transferase mutations wheat bx (SEQ ID No 4)

| Mutable amino | |
|---|---|
| acid position | |
| relative to | |
| SEQ ID NO: 4 | Substitution or addition* |
| 14(F) | M |
| 16(F) | Y |
| 17(L) | H, I, M, C, P, Q |
| 73(E) | M, L, I |
| 75(I) | F, Y |
| 76(A) | G, E, M, F, L, H, Q, N, S |
| 78(M) | W, C, V, I |
| 79(G) | A, C, P, V |
| 83(N) | D |
| 115(V) | L, I |
| 116(V) | T, C, I, S, V, G |
| 117(W) | Y, F |
| 134(I) | H, S, T, L, A, C, M, V |
| 135(M) | P |
| 137(A) | S |
| 142(F) | M, Y, K, L, F, W |
| 152(I) | T, Q, K, R, V, L, F, H |
| 180(L) | C, I, M |
| 190(F) | M, T, I, L |
| 193(L) | T, C, N, A, G, Q, I, V, D |
| 194(L) | I |
| 197(T) | V |
| 198(V) | M, N, H, Y |
| 209(I) | M, W, F |
| 219(N) | P, F, W, Y, H, K, L, M, S, T, R, G, C, I, E |
| 278(L) | V, W, F, I, M |
| 279(A) | V |
| 280(A) | C, Q, K, R, L, M, V, T, S |
| 333(S) | R, K, A |
| 362(I) | S, Q, W, A, V, L, F, T, M |
| 369(I) | S, T, N, H, F, T, A, V, G, Y |
| 371(H) | I, C |
| 375(G) | L, C, M |
| 432(A) | L, V, H, Q, P, T, F, Y, D, E, R, K, N |
| 437(G) | peptides, GIGVD, GIGVDV, GIGVDVD |
| | or GIGVDVDE |

*Unless otherwise denoted, the amino acids and peptides listed in this column represent some potential substitutions at the indicated position.

TABLE 5
Exemplary glucosyl transferase mutations
in Sorghum maize bx (SEQ ID No 5)

| Mutable amino | |
|---|---|
| acid position | |
| relative to | |
| SEQ ID NO: 5 | Substitution or addition* |
| 21(L) | M, F |
| 23(Y) | F, Y |
| 24(Q) | H, I, M, C, P |
| 80(K) | M, L, I, E |
| 82(I) | F, Y |
| 83(A) | G, E, M, F, L, H, Q, N, S |
| 85(V) | W, C, V, I |
| 86(V) | A, C, P |
| 90(N) | D |
| 120(A) | L, I, V |
| 121(V) | T, C, I, S, V, G |
| 122(W) | Y, F |
| 139(L) | H, S, T, I, L, A, C, M, V |
| 140(F) | P, M |
| 142(N) | S, A |
| 147(F) | M, Y, K, L, F, W |
| 157(I) | T, Q, K, R, V, L, F, H |
| 185(E) | C, I, M, L |
| 195(F) | M, T, I, L |
| 198(M) | T, C, N, A, G, Q, I, V, D, L |
| 199(V) | I, L |
| 202(V) | V, T |
| 203(V) | M, N, H, Y |
| 214(L) | F, M, W |
| 224(N) | P, F, W, Y, H, K, L, M, S, T, R, G, C, I, E |
| 281(I) | V, W, F, M |
| 282(A) | V |
| 283(A) | C, Q, K, R, L, M, V, T, S |
| 340(Y) | R, K, A |
| 369(I) | S, Q, W, A, V, F, T, M |
| 376(L) | S, T, N, H, F, T, A, I, G, V, Y |
| 378(R) | I, C |
| 382(G) | L, C, M |
| 439(A) | L, V, H, Q, P, T, F, Y, D, E, R, K, N |
| 444(T) | peptides, GIGVD, GIGVDV, GIGVDVD |
| | or GIGVDVDE |

*Unless otherwise denoted, the amino acids and peptides listed in this column represent some potential substitutions at the indicated position.

TABLE 6
Exemplary glucosyl transferase mutations
in barley bx (SEQ ID No 6)

| Mutable amino | |
|---|---|
| acid position | |
| relative to | |
| SEQ ID NO: 6 | Substitution or addition* |
| 18(L) | M, F |
| 20(Y) | Y, F |
| 21(Q) | H, I, M, C, P |
| 77(E) | M, L, I |
| 79(I) | F, Y |
| 80(A) | G, E, M, F, L, H, Q, N, S |
| 82(F) | W, C, V, I |
| 83(V) | A, C, P |
| 87(N) | D |
| 120(V) | L, I |
| 121(D) | T, C, I, V, S, G |
| 122(W) | Y, F |
| 139(L) | H, S, T, I, A, C, M, V |
| 140(M) | P |
| 142(T) | S, A |
| 147(F) | M, Y, K, L, F, W |
| 157(C) | T, Q, K, R, V, L, F, H, I |
| 187(D) | C, I, M, L |
| 198(Y) | M, T, I, L, F |
| 201(L) | T, C, N, A, G, Q, I, V, D |
| 202(L) | I |
| 205(I) | V, T |
| 206(V) | M, N, H, Y |
| 217(I) | M, W, F |
| 227(E) | P, F, W, Y, H, K, L, M, S, T, N, R, G, C, I, E |
| 285(L) | V, W, F, I, M |
| 286(V) | No change, A |
| 287(G) | C, Q, K, R, L, M, A, V, T, S |
| 340(S) | R, K, A |
| 369(I) | S, Q, W, A, V, L, F, T, M |
| 376(I) | S, T, N, H, F, T, A, V, G, Y |
| 378(R) | I, C |
| 382(G) | L, C, M |
| 439(A) | L, V, H, Q, P, T, F, Y, D, E, R, K, N |
| 444(S) | peptides, GIGVD, GIGVDV, GIGVDVD |
| | or GIGVDVDE |

*Unless otherwise denoted, the amino acids and peptides listed in this column represent some potential substitutions at the indicated position.

TABLE 7
Exemplary glucosyl transferase mutations
in Alopecurus bx (SEQ ID No 7)

| Mutable amino | |
|---|---|
| acid position | |
| relative to | |
| SEQ ID NO: 7 | Substitution or addition* |
| 18(L) | M, F |
| 20(Y) | Y, F |
| 21(Q) | H, I, M, C, P |
| 76(L) | M, E, I |
| 78(V) | F, Y, I |
| 79(M) | G, E, F, L, H, Q, N, S, A |
| 81(H) | W, C, V |
| 82(V) | A, C, P |
| 86(N) | D |
| 117(A) | L, I, V |
| 118(H) | T, C, I, V, S, G |
| 119(L) | Y, F, W |
| 136(L) | H, S, T, I, A, C, M, V |
| 137(R) | P, M |
| 139(G) | S, A |
| 144(F) | M, Y, K, L, F, W |
| 154(C) | T, Q, K, R, V, L, F, H, I |
| 182(M) | C, I, L, M |
| 194(S) | M, T, I, L, F |
| 197(L) | T, C, N, A, G, Q, I, V, D |
| 198(L) | I, T |
| 201(A) | V, T |
| 202(V) | M, N, H, Y |
| 213(L) | M, W, F |
| 223(D) | P, F, W, Y, H, K, L, M, S, N, R, G, C, I, T, E |
| 282(L) | V, W, F, I, M |
| 283(A) | V |
| 284(S) | C, Q, K, R, L, M, A, V, T |
| 337(S) | R, K, A |
| 366(I) | S, Q, W, A, V, L, F, T, M |
| 373(I) | S, T, N, H, F, T, A, V, G, Y |
| 375(R) | I, C |
| 379(A) | L, C, M, G |
| 434(A) | L, V, H, Q, P, T, F, Y, D, E, R, K, N |
| 439(K) | peptides, GIGVD, GIGVDV, GIGVDVD |
| | or GIGVDVDE |

*Unless otherwise denoted, the amino acids and peptides listed in this column represent some potential substitutions at the indicated position.

TABLE 8
Exemplary glucosyl transferase mutations
in Avena bx9 (SEQ ID No 8)

| Mutable amino | |
|---|---|
| acid position | |
| relative to | |
| SEQ ID NO: 8 | Substitution or addition* |
| 19(L) | F, M |
| 21(F) | Y |
| 22(Q) | H, I, M, C, P |
| 77(G) | M, L, I, D, E |
| 79(I) | F, Y |
| 80(I) | G, E, M, F, L, H, Q, N, S, A |
| 82(I) | W, C, V |
| 83(I) | A, C, P, V |
| 87(N) | D |
| 116(A) | L, I, V |
| 117(N) | T, C, I, V, S, G |
| 118(L) | Y, F, W |
| 135(L) | H, S, T, I, A, C, M, V |
| 136(R) | P, M |
| 138(G) | S, A |
| 143(F) | M, Y, K, L, F, W |
| 153(H) | T, Q, K, R, V, L, F, I |
| 181(F) | C, I, M, L |
| 191(V) | M, T, I, L, F |
| 194(V) | T, C, N, A, G, Q, I, L, D |
| 195(L) | I |
| 198(A) | V, T |
| 199(T) | M, N, H, Y, V |
| 210(I) | M, W, F |
| 220(E) | P, F, W, Y, H, K, L, M, S, N, R, G, C, I, T |
| 278(L) | V, W, F, I, M |
| 279(A) | V |
| 280(S) | C, Q, K, R, L, M, A, V, T |
| 333(P) | R, K, A |
| 362(I) | S, Q, W, A, V, L, F, T, M |
| 369(I) | S, T, N, H, F, T, A, V, G, Y |
| 371(R) | I, C |
| 375(A) | L, C, M, G |
| 430(A) | L, V, H, Q, P, T, F, Y, D, E, R, K, N |
| 435(E) | peptides, GIGVD, GIGVDV, GIGVDVD |
| | or GIGVDVDE |

*Unless otherwise denoted, the amino acids and peptides listed in this column represent some potential substitutions at the indicated position.

TABLE 9
Exemplary glucosyl transferase mutations
in rice bx (Q53K20 SEQ ID No 9)

| Mutable amino | |
|---|---|
| acid position | |
| relative to | |
| SEQ ID NO: 9 | Substitution or addition* |
| 13(M) | M, F |
| 15(Y) | Y, F |
| 16(P) | H, I, M, C, Q |
| 72(E) | M, L, I |
| 74(A) | F, Y, I |
| 75(A) | G, E, M, F, L, H, Q, N, S |
| 77(V) | W, C, I |
| 78(L) | A, C, P, V |
| 82(N) | D |
| 110(V) | L, I |
| 111(M) | T, C, I, V, S, G |
| 112(W) | Y, F |
| 129(L) | H, S, T, I, M, A, C, V |
| 130(M) | P |
| 132(S) | A |
| 137(F) | M, Y, K, L, F, W |
| 147(L) | T, Q, K, R, V, I, F, H |
| 175(Q) | C, I, M, L |
| 185(F) | M, T, I, L |
| 188(V) | T, C, N, A, G, Q, I, L, D |
| 189(L) | I |
| 192(V) | V, T |
| 193(V) | M, N, H, Y |
| 204(L) | M, W, F |
| 214(N) | P, F, W, Y, H, K, L, M, S, T, R, G, C, I, E |
| 271(M) | V, W, F, I |
| 272(A) | V |
| 273(I) | C, Q, K, R, L, M, A, V, T, S |
| 328(S) | R, K, A |
| 357(I) | S, Q, W, A, V, L, F, T, M |
| 364(I) | S, T, N, H, F, T, A, V, G, Y |
| 366(R) | I, C |
| 370(G) | L, C, M |
| 427(A) | L, V, H, Q, P, T, F, Y, D, E, R, K, N |
| 432(S) | peptides, GIGVD, GIGVDV, GIGVDVD |
| | or GIGVDVDE |

*Unless otherwise denoted, the amino acids and peptides listed in this column represent some potential substitutions at the indicated position.

The terms “polypeptide,” “peptide,” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. Polypeptides of the invention can be produced either from a nucleic acid disclosed herein, or by the use of standard molecular biology techniques. For example, a truncated protein of the invention can be produced by expression of a recombinant nucleic acid of the invention in an appropriate host cell, or alternatively by a combination of ex vivo procedures, such as protease digestion and purification. Accordingly, the present invention also provides nucleic acid molecules comprising polynucleotide sequences that encode glucosyl transferase polypeptides having at least about 60%, 65%, 70%, 75%, 80%, 85%, 90%, 91%, 92%, 93%, 94%, 95%, 96%, 97%, 98%, 99% or more sequence identity to a sequence selected from the group consisting of : SEQ ID NO:1 (bx9), SEQ ID NO:2 (bx8), SEQ ID NO:3 (Echinocloa), SEQ ID NO:4 (wheat), SEQ ID NO:5 (sorghum), SEQ ID NO:6 (barley), SEQ ID NO:7 (Alopecurus) SEQ ID NO:8 (Avena) and SEQ ID NO:9 (rice) as well as variants and fragments thereof capable of exhibiting glucosyl transferase enzymatic activity in respect of certain herbicides selected from the group consisting of structures: III, IV, V, VI, VII, VIII, IX, X, XI, XII and metribuzin. The present invention also provides nucleic acid molecules that encode certain mutant glucosyl transferase polypeptides having at least 60%, 65%, 70%, 75%, 80%, 85%, 90%, 91%, 92%, 93%, 94%, 95%, 96%, 97%, 98%, 99% or more sequence identity to a sequence selected from the group consisting of : SEQ ID NO:1 (bx9), SEQ ID NO:2 (bx8), SEQ ID NO:3 (Echinocloa), SEQ ID NO:4 (wheat), SEQ ID NO:5 (sorghum), SEQ ID NO:6 (barley), SEQ ID NO:7 (Alopecurus) SEQ ID NO:8 (Avena) and SEQ ID NO:9 (rice) that are capable of catalyzing the transfer of glucose from UDP glucose to a herbicide selected from the group consisting of structures: III, IV, V, VI, VII, VIII, IX, X, XI, XII and metribuzin wherein, relative to the wild type, the said polypeptide comprises one or more of the amino acid substitutions selected from the group that is set out elsewhere herein.

In general, the invention also includes any polynucleotide sequence that encodes any of the mutant glucosyl transferase polypeptides described herein, as well as any polynucleotide sequence that encodes glucosyl transferase polypeptides having one or more conservative amino acid substitutions relative to the mutant glucosyl transferase polypeptides described herein. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine I, Lysine (K), Histidine (H); Acidic: Aspartic acid (D), Glutamic acid (E), Asparagine (N), Glutamine (Q).

In one embodiment, the present invention provides a polynucleotide sequence encoding an amino acid sequence having at least about 60%, 65%, 70%, 75%, 80%, 85%, 90%, 91%, 92%, 93%, 94%, 95%, 96%, 97%, 98%, 99% or more sequence identity to SEQ ID NO:1 or to SEQ ID NO:2 or to SEQ ID NO:3 or to SEQ ID NO:4 or to SEQ ID NO:5 or to SEQ ID NO:6 or to SEQ ID NO:7 or to SEQ ID NO:8 or to SEQ ID NO:9 where the glucosyl transferase amino acid sequence derives from a plant, where the polypeptide has enzymatic activity, and where the polypeptide contains one or more substitutions, additions or deletions as discussed infra. In particular embodiments, the polynucleotide sequence encodes a mutant glucosyl transferase polypeptide having an amino acid sequence selected from the group consisting of SEQ IDs NO: 16-54.

As used herein, “nucleic acid” includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to naturally occurring nucleotides.

As used herein, the terms “encoding” or “encoded” when used in the context of a specified nucleic acid mean that the nucleic acid comprises the requisite information to direct translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

The invention encompasses isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of interfering enzyme activities and that is capable being characterized in respect of its catalytic, kinetic and molecular properties includes quite crude preparations of protein (for example recombinantly produced in cell extracts) having less than about 98%, 95% 90%, 80%, 70%, 60% or 50% (by dry weight) of contaminating protein as well as preparations further purified by methods known in the art to have 40%, 30%, 20%, 10%, 5%, or 1% (by dry weight) of contaminating protein.

The proteins of the invention may be altered in various ways including amino acid substitutions, deletions, truncations, and insertions. Methods for such manipulations are generally known in the art. For example, amino acid sequence variants and fragments of the mutant glucosyl transferase proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that often do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The polynucleotides of the invention can also be used to isolate corresponding sequences from other organisms, particularly other plants. In this manner, methods such as PCR, hybridization, and the like can be used to identify such sequences based on their sequence homology to the sequences set forth herein.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art. See, for example, Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

By “hybridizing to” or “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

Fragments and variants of the disclosed nucleotide sequences and proteins encoded thereby are also encompassed by the present invention. “Fragment” is intended to mean a portion of the nucleotide sequence or a portion of the amino acid sequence and hence protein encoded thereby. Fragments of a nucleotide sequence may encode protein fragments that retain the biological activity of the mutant glucosyl transferase protein and hence have glucosyl transferase enzymatic activity. Alternatively, fragments of a nucleotide sequence that are useful as hybridization probes or in mutagenesis and shuffling reactions to generate yet further glucosyl transferase variants generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the polypeptides of the invention.

A fragment of a nucleotide sequence that encodes a biologically active portion of a mutant glucosyl transferase protein of the invention will encode at least 15, 25, 30, 40, 50, 60, 70, 80, 90, 100, 110, 120, 150, 180, 200, 250, 300, 350 contiguous amino acids, or up to the total number of amino acids present in a full-length mutant glucosyl polypeptide of the invention. Fragments of a nucleotide sequence that are useful as hybridization probes or PCR primers generally need not encode a biologically active portion of a glucosyl transferase protein.

As used herein, “full-length sequence” in reference to a specified polynucleotide means having the entire nucleic acid sequence of a native or mutated glucosyl transferase sequence. “Native sequence” is intended to mean an endogenous sequence, i.e., a non-engineered sequence found in an organism's genome.

Thus, a fragment of a nucleotide sequence of the invention may encode a biologically active portion of a mutant glucosyl transferase polypeptide, or it may be a fragment that can be used as a hybridization probe etc. or PCR primer using methods disclosed below. A biologically active portion of a mutant glucosyl transferase polypeptide can be prepared by isolating a portion of one of the nucleotide sequences of the invention, expressing the encoded portion of the mutant glucosyl transferase protein (e.g., by recombinant expression in vitro), and assessing the activity of the encoded portion of the mutant glucosyl transferase protein. Nucleic acid molecules that are fragments of a nucleotide sequence of the invention comprise at least 15, 20, 50, 75, 100, 150, 200, 300, 400, 500, 600, 700, 800, 900, 1000, 1100, 1200, or 1300 contiguous nucleotides, or up to the number of nucleotides present in a full-length nucleotide sequence disclosed herein.

“Variants” is intended to mean substantially similar sequences. For polynucleotides, a variant comprises a deletion and/or addition of one or more nucleotides at one or more internal sites within the reference polynucleotide and/or a substitution of one or more nucleotides at one or more sites in the mutant glucosyl transferase polynucleotide. As used herein, a “reference” polynucleotide or polypeptide comprises a glucosyl transferase nucleotide sequence or amino acid sequence, respectively. As used herein, a “native” polynucleotide or polypeptide comprises a naturally occurring nucleotide sequence or amino acid sequence, respectively. One of skill in the art will recognize that variants of the nucleic acids of the invention will be constructed such that the open reading frame is maintained. For polynucleotides, conservative variants include those sequences that, because of the degeneracy of the genetic code, encode the amino acid sequence of one of the mutant glucosyl transferase polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode a mutant glucosyl transferase protein of the invention. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptides of SEQ ID NOS: 1-14 and 32-49 is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity across the entirety of the glucosyl transferase sequences described herein.

“Variant” protein is intended to mean a protein derived from the reference protein by deletion or addition of one or more amino acids at one or more internal sites in the glucosyl transferase protein and/or substitution of one or more amino acids at one or more sites in the glucosyl transferase protein. Variant proteins encompassed by the present invention are biologically active, that is they continue to possess the desired biological activity of the glucosyl transferase protein, that is, glucosyl transferase enzymatic activity as described herein. Such variants may result from, for example, genetic polymorphism or from human manipulation. Biologically active variants of a mutant glucosyl transferase protein of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity across the entirety of the amino acid sequence for the mutant glucosyl transferase protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

Methods of alignment of sequences for comparison are well known in the art and can be accomplished using mathematical algorithms such as the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; and the algorithm of Karlin and Altschul (1990) Proc. Natl. Acad. Sci. USA 872264, modified as in Karlin and Altschul (1993) Proc. Natl. Acad. Sci. USA 90:5873-5877. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA).

### Genome Editing

As an alternative to the use of a transgene, the herbicide tolerance trait associated with expression of the mutant glucosyl transferase polypeptide sequences of the current invention may be obtained via genome editing and/or mutagenesis technologies that are well known in the art. As well, introduction may be accomplished by any manner known in the art, including: introgression, transgenic, or site-directed nucleases (SDN). Particularly, the modification to the nucleic acid sequence is introduced by way of site-directed nuclease (SDN). More particularly, the SDN is selected from: meganuclease, zinc finger, transcription activator-like effector nucleases system (TALEN) or Clustered Regularly Interspaced Short Palindromic Repeats system (CRISPR) system.

SDN is also referred to as “genome editing”, or genome editing with engineered nucleases (GEEN). This is a type of genetic engineering in which DNA is inserted, deleted or replaced in the genome of an organism using engineered nucleases that create site-specific double-strand breaks (DSBs) at desired locations in the genome. The induced double-strand breaks are repaired through nonhomologous end-joining (NHEJ) or homologous recombination (HR), resulting in targeted mutations (‘edits’). Particularly SDN may comprises techniques such as: Meganucleases, Zinc finger nucleases (ZFNs), Transcription Activator-Like Effector-based Nucleases (TALEN) (Joung & Sander 2013), and the Clustered Regularly Interspaced Short Palindromic Repeats (CRISPR-Cas) system.

Most particularly, introduction of the nucleic acid is accomplished by heterologous or transgenic gene expression. For example, as well as random mutagenesis, directed methods using chimeric oligonucleotide-directed repair mutagenesis, CRISPR, TALEN or Zinc finger technology and similar technologies designed to produce DNA strand breaks at directed positions and thereby to induce mutations and/or specific insertions of DNA via homologous recombination are now available. These methods provide ways of targeting mutagenesis to a particular endogenous gene of choice (which for the current example might be, for example, the bx9 gene of maize in maize crop plants) so as to obtain desirable mutations and therefore expression of desirable mutant proteins in plant cells (which, in the current context, means the mutant glucosyl transferase polypeptides described herein).

In particular embodiments one or more of the mutations of the current invention (see Tables 1 to 9) are, for example, directly introduced into the endogenous bx gene sequences of various crops such as maize, wheat, barley, rye, rice and sorghum. For example, in one particular embodiment regenerable maize callus is genome edited by CRISPR so, for example, as to introduce the desired mutational changes A334R, S117V and M279F into the endogenous maize bx9 gene (which encodes the polypeptide of SEQ ID No 1) in order to regenerate plantlets selectable and useful on the basis of their improved herbicide tolerance to certain alcohol and aminal PSII herbicides. Alternatively, in other embodiments, CRISPR genome editing is used to generate corn having, for example, a M279F, A432P double mutation or a M279W, A432F, S117G, F19M quadruple mutation in the endogenous maize gene in order to regenerate plantlets selectable and useful on the basis of their improved tolerance to the amine herbicide, metribuzin.

Similarly the same methods of directed mutagenesis may also be used to further genome edit transgenic seeds, callus and plants that are the product of application of methods of the current invention so as to add yet further desired mutations to transgenic events in crops. Such mutations may optionally introduce mutations (or additional mutations) into the glucosyl transferase genes of the current invention and be similarly directed toward improving herbicide tolerance or be directed to other genes and directed to the improvement of other traits or aspects of plant performance.

### Gene Stacking

In certain embodiments the polynucleotides of the invention encoding polypeptides with glucosyl transferase to an amine, alcohol or aminal herbicide (e.g., a polynucleotide sequence encoding an amino acid sequence selected from the group consisting of SEQ ID NO:1-54) are stacked with any combination of polynucleotide sequences of interest in order to create plants with a combination of desired traits. A trait, as used herein, refers to the phenotype derived from a particular sequence or groups of sequences. For example, a polynucleotide which encodes a mutant glucosyl transferase polypeptide or variant thereof with herbicide glucosyl transferase enzymatic activity may be stacked with any other polynucleotide or polynucleotides encoding polypeptides that confer a desirable trait, including but not limited to resistance to diseases, insects, further herbicide tolerances, tolerance to heat and drought, reduced time to crop maturity, improved industrial processing, such as for the conversion of starch or biomass to fermentable sugars, and improved agronomic quality, such as high oil content and high protein content.

Exemplary polynucleotides that may be stacked with polynucleotides of the current invention include polynucleotides encoding polypeptides conferring resistance to pests/pathogens such as viruses, nematodes, insects or fungi, and the like. Exemplary polynucleotides that may be stacked with polynucleotides of the invention include polynucleotides encoding: polypeptides having pesticidal and/or insecticidal activity, such as other Bacillus thuringiensis toxic proteins (described in U.S. Pat. Nos. 5,366,892; 5,747,450; 5,737,514; 5,723,756; 5,593,881; and Geiser et al. (1986) Gene 48:109), lectins (Van Damme et al. (1994) Plant Mol. Biol. 24:825, pentin (described in U.S. Pat. No. 5,981,722), and the like; traits desirable for disease or herbicide resistance (e.g., fumonisin detoxification genes (U.S. Pat. No. 5,792,931; resistance to HPPD inhibitor herbicides e.g. WO 2010/085705; WO 2011/068567); resistance to protoporphyrinogen oxidase-inhibiting herbicides e.g. WO15092706; WO2010143743, avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; glyphosate resistance (e.g., 5-enol-pyrovyl-shikimate-3-phosphate-synthase (EPSPS) gene, described in U.S. Pat. Nos. 4,940,935 and 5,188,642; or the glyphosate N-acetyltransferase (GAT) gene, described in Castle et al. (2004) Science, 304:1151-1154; and in U.S. Patent App. Pub. Nos. 20070004912, 20050246798, and 20050060767)); glufosinate resistance (e.g., phosphinothricin acetyl transferase genes PAT and BAR, described in U.S. Pat. Nos. 5,561,236 and 5,276,268); a cytochrome P450 or variant thereof that confers herbicide resistance or tolerance to, inter alia, HPPD herbicides (U.S. patent application Ser. No. 12/156,247; U.S. Pat. Nos. 6,380,465; 6,121,512; 5,349,127; 6,649,814; and 6,300,544; and PCT Patent App. Pub. No. WO2007000077); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5.602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)); the disclosures of which are herein incorporated by reference.

Thus, in one embodiment, the polynucleotide encoding a polypeptide with glucosyl transferase to an amine, alcohol or aminal herbicide is stacked with one or more polynucleotides encoding polypeptides that confer resistance or tolerance to one or more further herbicides. In a particular such embodiment, the desirable stack of traits is resistance or tolerance to an amine, alcohol or aminal PSII herbicide combined with resistance to an HPPD herbicide. In another embodiment, the desirable stack of traits is resistance or tolerance to an amine, alcohol or aminal PSII herbicide combined with resistance to glyphosate and/or to one or more auxin herbicides and/or to one or more protoporphyrinogen oxidase inhibitor herbicides. In a further embodiment, the amine, alcohol or aminal PSII resistance trait is stacked with resistance to an auxin herbicide and/or with resistance or tolerance to glufosinate.

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

Alternatively, the herbicide tolerance trait based on expression of the mutant glucosyl transferase polypeptide sequences described herein may be obtained in a plant via genome editing and directed in situ mutagenesis using, for example, chimeric oligonucleotides, CRISPR, TALEN or Zn finger technology as described in the various patents and patent applications which are incorporated herein. Similarly many of the herbicide tolerances, e.g. to ALS or ACCase herbicides that may optionally be stacked with the glucosyl transferases of the current invention may themselves also be derived via random or directed in situ mutagenesis of the plant genome rather than be conferred by a transgene.

### Plant Expression Cassettes

The compositions of the invention may additionally contain nucleic acid sequences for transformation and expression in a plant of interest. The nucleic acid sequences may be present in DNA constructs or expression cassettes. “Expression cassette” as used herein means a nucleic acid molecule capable of directing expression of a particular nucleotide sequence in an appropriate host cell, comprising a promoter operatively linked to the nucleotide sequence of interest (i.e., a polynucleotide encoding a mutant glucosyl transferase polypeptide or variant thereof that retains glucosyl transferase enzymatic activity, alone or in combination with one or more additional nucleic acid molecules encoding polypeptides that confer desirable traits) which is operatively linked to termination signals. It also typically comprises sequences required for proper translation of the nucleotide sequence. The coding region usually codes for a protein of interest but may also code for a functional RNA of interest, for example antisense RNA or a non-translated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. Additionally, the promoter can also be specific to a particular tissue or organ or stage of development.

The present invention encompasses the transformation of plants with expression cassettes capable of expressing a polynucleotide of interest, i.e., a polynucleotide encoding a mutant glucosyl transferase polypeptide or variant thereof that retains glucosyl transferase enzymatic activity in respect of certain herbicide classes, alone or in combination with one or more additional nucleic acid molecules encoding polypeptides that confer desirable traits. The expression cassette will include in the 5′-3′ direction of transcription, a transcriptional and translational initiation region (i.e., a promoter) and a polynucleotide open reading frame. The expression cassette may optionally comprise a transcriptional and translational termination region (i.e. termination region) functional in plants. In some embodiments, the expression cassette comprises a selectable marker gene to allow for selection for stable transformants. Expression constructs of the invention may also comprise a leader sequence and/or a sequence allowing for inducible expression of the polynucleotide of interest. See, Guo et al. (2003) Plant J. 34:383-92 and Chen et al. (2003) Plant J. 36:731-40 for examples of sequences allowing for inducible expression.

The regulatory sequences of the expression construct are operably linked to the polynucleotide of interest. By “operably linked” is intended a functional linkage between a promoter and a second sequence wherein the promoter sequence initiates and mediates transcription of the DNA sequence corresponding to the second sequence. Generally, operably linked means that the nucleotide sequences being linked are contiguous.

Any promoter capable of driving expression in the plant of interest may be used in the practice of the invention. The promoter may be native or analogous or foreign or heterologous to the plant host. The terms “heterologous” and “exogenous” when used herein to refer to a nucleic acid sequence (e.g. a DNA or RNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA or RNA) sequence naturally associated with a host cell into which it is introduced.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to that sequence. The promoters that are used for expression of the transgene(s) can be a strong plant promoter, a viral promoter, or a chimeric promoters composed of elements such as: TATA box from any gene (or synthetic, based on analysis of plant gene TATA boxes), optionally fused to the region 5′ to the TATA box of plant promoters (which direct tissue and temporally appropriate gene expression), optionally fused to 1 or more enhancers (such as the 35S enhancer, FMV enhancer, CMP enhancer, RUBISCO SMALL SUBUNIT enhancer, PLASTOCYANIN enhancer).

Exemplary constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Appropriate plant or chimeric promoters are useful for applications such as expression of transgenes in certain tissues, while minimizing expression in other tissues, such as seeds, or reproductive tissues. Exemplary cell type- or tissue-preferential promoters drive expression preferentially in the target tissue, but may also lead to some expression in other cell types or tissues as well. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

In other embodiments of the present invention, inducible promoters may be desired. Inducible promoters drive transcription in response to external stimuli such as chemical agents or environmental stimuli. For example, inducible promoters can confer transcription in response to hormones such as gibberellic acid or ethylene, or in response to light or drought.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tml terminator, the nopaline synthase terminator and the pea rbcs E9 terminator. These can be used in both monocotyledons and dicotyledons. In addition, a gene's native transcription terminator may be used.

Generally, the expression cassette will comprise a selectable marker gene for the selection of transformed cells. Selectable marker genes are utilized for the selection of transformed cells or tissues.

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize Adhl gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1:1183-1200 (1987)). In the same experimental system, the intron from the maize bronze 1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picomavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Samow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

The present invention also relates to nucleic acid constructs comprising one or more of the expression cassettes described above. The construct can be a vector, such as a plant transformation vector. In some preferred embodiments, the vector is a plant transformation vector comprising a polynucleotide encoding the polypeptide sequences set forth in SEQ ID NO: 16, 17, 18, 19, 20, 21, 22, 23, 24, 25, 26, 27, 28, 29 or 30.

### Plants

As used herein, the term “plant part” or “plant tissue” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like.

Plants useful in the present invention include plants that are transgenic for a polynucleotide encoding a polypeptide with glucosyl transferase activity to an amine, alcohol or aminal PSII herbicide where this polynucleotide may be present alone or in combination with one or more additional nucleic acid molecules encoding polypeptides that confer further desirable traits. Plants useful in the present invention further include plants with mutations in an endogenous glucosyl transferase gene leading to expression of a mutant glucosyl transferase polypeptide or variant thereof that confers improved glucosyl transferase to an amine, alcohol or aminal PSII herbicide where these mutations may be present alone in a plant or in combination with one or more additional nucleic acid molecules or further mutations encoding polypeptides that confer further desirable and/or improved traits. The type of plant selected depends on a variety of factors, including for example, the downstream use of the harvested plant material, amenability of the plant species to transformation, and the conditions under which the plants will be grown, harvested, and/or processed. One of skill will further recognize that additional factors for selecting appropriate plant varieties for use in the present invention include high yield potential, good stalk strength, resistance to specific diseases, drought tolerance, rapid dry down and grain quality sufficient to allow storage and shipment to market with minimum loss.

Plants according to the present invention include any plant that is cultivated for the purpose of producing plant material that is sought after by man or beast for either oral consumption, or for utilization in an industrial, pharmaceutical, or commercial process. The invention may be applied to any of a variety of plants, including, but not limited to maize, wheat, rice, barley, soybean, cotton, sorghum, beans in general, rape/canola, alfalfa, flax, mangelwurzels, sunflower, safflower, millet, rye, sugarcane, sugar beet, cocoa, tea, Brassica, cotton, coffee, sweet potato, flax, peanut, clover; vegetables such as lettuce, tomato, cucurbits, cassava, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, and coconut; and flowers such as orchids, carnations and roses. Other plants useful in the practice of the invention include perennial grasses, such as switchgrass, prairie grasses, Indiangrass, Big bluestem grass and the like. It is recognized that mixtures of plants may be used.

In addition, the term “crops” is to be understood as also including crops that have been rendered tolerant to herbicides or classes of herbicides (such as, for example, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors) as a result of conventional methods of breeding or genetic engineering. Examples of crops that have been rendered tolerant to herbicides or classes of herbicides by genetic engineering methods include glyphosate- and glufosinate-resistant crop varieties commercially available under the trade names RoundupReady® and LibertyLink®. The method according to the present invention is especially suitable for the protection of soybean crops or of maize crops which have also been rendered tolerant to glyphosate and/or glufosinate and where these herbicides are used in a weed control program along with other herbicides (e.g. HPPD herbicides) but where it is desirable to also further use a potent PSII herbicide in order to provide more complete weed control and/or to control resistant biotypes.

It is further contemplated that the constructs of the invention may be introduced into plant varieties having improved properties suitable or optimal for a particular downstream use. For example, naturally-occurring genetic variability results in plants with resistance or tolerance to PSII inhibitors or other herbicides, and such plants are also useful in the methods of the invention. The method according to the present invention can be further optimized by crossing the transgenes that provide a level of tolerance, with soybean and maize cultivars that exhibit an enhanced level of tolerance to PSII inhibitors that is found in a small percentage of lines.

### Plant Transformation

Once an herbicide-resistance conferring glucosyl transferase polynucleotide, alone or in combination with one or more additional nucleic acid molecules encoding polypeptides that confer desirable traits, has been cloned into an expression system, it is transformed into a plant cell. The receptor and target expression cassettes of the present invention can be introduced into the plant cell in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide is intended to mean that a polynucleotide is introduced into the plant and does not integrate into the genome of the plant.

By “stably introducing” or “stably introduced” in the context of a polynucleotide introduced into a plant is intended the introduced polynucleotide is stably incorporated into the plant genome, and thus the plant is stably transformed with the polynucleotide.

“Stable transformation” or “stably transformed” is intended to mean that a polynucleotide, for example, a nucleotide construct described herein, introduced into a plant integrates into the genome of the plant and is capable of being inherited by the progeny thereof, more particularly, by the progeny of multiple successive generations.

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptll gene, which confers resistance to kanamycin and related antibiotics (Messing & Vierra Gene 19: 259-268 (1982); Bevan et al., Nature 304:184-187 (1983)), the pat and bar genes, which confer resistance to the herbicide glufosinate (also called phosphinothricin; see White et al., Nucl. Acids Res 18: 1062 (1990), Spencer et al. Theor. Appl. Genet 79: 625-631 (1990) and U.S. Pat. Nos. 5,561,236 and 5,276,268), the hph gene, which confers resistance to the antibiotic hygromycin (Blochinger & Diggelmann, Mol. Cell Biol. 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), the glyphosate N-acetyltransferase (GAT) gene, which also confers resistance to glyphosate (Castle et al. (2004) Science, 304:1151-1154; U.S. Patent App. Pub. Nos. 20070004912, 20050246798, and 20050060767); and the mannose-6-phosphate isomerase gene, which provides the ability to metabolize mannose (U.S. Pat. Nos. 5,767,378 and 5,994,629). Alternatively, and in one preferred embodiment the glucosyl transferase gene of the current invention is, in combination with the use of a suitable substrate PSII herbicide as selection agent, itself used as the selectable marker.

Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens. These typically carry at least one T-DNA border sequence and include vectors such as pBIN19 (Bevan, Nucl. Acids Res. (1984)). For the construction of vectors useful in Agrobacterium transformation, see, for example, US Patent Application Publication No. 2006/0260011, herein incorporated by reference.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. For the construction of such vectors, see, for example, US Application No. 20060260011, herein incorporated by reference.

For expression of a nucleotide sequence of the present invention in plant plastids, plastid transformation vector pPH143 (WO 97/32011, See Example 36) is used. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J. 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both of these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8:736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology 11:1553-1558 (1993)) and Weeks et al. (Plant Physiol. 102:1077-1084 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSOG35) is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 hours (still on osmoticum). After 24 hours, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference.

For example, rice (Oryza sativa) can be used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in quantity or substituted. Embryogenic responses are initiated and/or cultures are established from mature embryos by culturing on MS-CIM medium (MS basal salts, 4.3 g/liter; B5 vitamins (200×), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for about2 days at 28° C. Agrobacterium is re-suspended in liquid MS-CIM medium. The Agrobacterium culture is diluted to an OD600 of 0.2-0.3 and acetosyringone is added to a final concentration of 200 uM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2, 4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (To generation) grown to maturity, and the T₁ seed is harvested.

The plants obtained via transformation with a nucleic acid sequence of interest in the present invention can be any of a wide variety of plant species, including those of monocots and dicots; however, the plants used in the method of the invention are preferably selected from the list of agronomically important target crops set forth elsewhere herein. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

For the transformation of plastids, seeds of Nicotiana tabacum c.v. “Xanthienc” are germinated seven per plate in a 1″ circular array on T agar medium and bombarded 12-14 days after sowing with 1 um tungsten particles (M10, Biorad, Hercules, Calif.) coated with DNA from plasmids pPH143 and pPH145 essentially as described (Svab, Z. and Maliga, P. (1993) PNAS 90, 913-917). Bombarded seedlings are incubated on T medium for two days after which leaves are excised and placed abaxial side up in bright light (350-500 umol photons/m²/s) on plates of RMOP medium (Svab, Z., Hajdukiewicz, P. and Maliga, P. (1990) PNAS 87, 8526-8530) containing 500 ug/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346349) is separated on 1% Tris-borate (TBE) agarose gels, transferred to nylon membranes (Amersham) and probed with .sup.32P-labeled random primed DNA sequences corresponding to a 0.7 kb BamHI/HindIII DNA fragment from pC8 containing a portion of the rps 7/12plastid targeting sequence. Homoplasmic shoots are rooted aseptically on spectinomycin-containing MS/IBA medium (McBride, K. E. et al. (1994) PNAS 91, 7301-7305) and transferred to the greenhouse.

The genetic properties engineered into the genome-edited or transgenic seeds and plants described above are passed on by sexual reproduction or vegetative growth and can thus be maintained and propagated in progeny plants. Generally, maintenance and propagation make use of known agricultural methods developed to fit specific purposes such as tilling, sowing or harvesting.

Use of the advantageous genetic properties of the genome-edited or transgenic plants and seeds according to the invention can further be made in plant breeding. Depending on the desired properties, different breeding measures are taken. The relevant techniques are well known in the art and include but are not limited to hybridization, inbreeding, backcross breeding, multi-line breeding, variety blend, interspecific hybridization, aneuploid techniques, etc. Thus, the genome edited or transgenic seeds and plants according to the invention can be used for the breeding of improved plant lines that, for example, increase the effectiveness of conventional methods such as herbicide or pesticide treatment or allow one to dispense with said methods due to their modified genetic properties.

Many suitable methods for transformation using suitable selection markers such as kanamycin, binary vectors such as from Agrobacterium and plant regeneration as, for example, from tobacco leaf discs are well known in the art.

### Herbicide Resistance

The present invention provides genome-edited and transgenic plants, plant cells, tissues, and seeds that have been mutated or transformed with a nucleic acid molecule to express a mutant glucosyl transferase or variant thereof that confers resistance or tolerance to herbicides, alone or in combination with one or more additional nucleic acid molecules encoding polypeptides that confer desirable traits.

In one embodiment, the genome edited or transgenic plants of the invention exhibit resistance or tolerance to application of herbicide in an amount of from about 5 to about 2,000 grams per hectare (g/ha), including, for example, about 5 g/ha, about 10 g/ha, about 15 g/ha, about 20 g/ha, about 25 g/ha, about 30 g/ha, about 35 g/ha, about 40 g/ha, about 45 g/ha, about 50 g/ha, about 55 g/ha, about 60 g/ha, about 65 g/ha, about 70 g/ha, about 75 g/ha, about 80 g/ha, about 85 g/ha, about 90 g/ha, about 95 g/ha, about 100 g/ha, about 110 g/ha, about 120 g/ha, about 130 g/ha, about 140 g/ha, about 150 g/ha, about 160 g/ha, about 170 g/ha, about 180 g/ha, about 190 g/ha, about 200 g/ha, about 210 g/ha, about 220 g/ha, about 230 g/ha, about 240 g/ha, about 250 g/ha, about 260 g/ha, about 270 g/ha, about 280 g/ha, about 290 g/ha, about 300 g/ha, about 310 g/ha, about 320 g/ha, about 330 g/ha, about 340 g/ha, about 350 g/ha, about 360 g/ha, about 370 g/ha, about 380 g/ha, about 390 g/ha, about 400 g/ha, about 410 g/ha, about 420 g/ha, about 430 g/ha, about 440 g/ha, about 450 g/ha, about 460 g/ha, about 470 g/ha, about 480 g/ha, about 490 g/ha, about 500 g/ha, about 510 g/ha, about 520 g/ha, about 530 g/ha, about 540 g/ha, about 550 g/ha, about 560 g/ha, about 570 g/ha, about 580 g/ha, about 590 g/ha, about 600 g/ha, about 610 g/ha, about 620 g/ha, about 630 g/ha, about 640 g/ha, about 650 g/ha, about 660 g/ha, about 670 g/ha, about 680 g/ha, about 690 g/ha, about 700 g/ha, about 710 g/ha, about 720 g/ha, about 730 g/ha, about 740 g/ha, about 750 g/ha, about 760 g/ha, about 770 g/ha, about 780 g/ha, about 790 g/ha, about 800 g/ha, about 810 g/ha, about 820 g/ha, about 830 g/ha, about 840 g/ha, about 850 g/ha, about 860 g/ha, about 870 g/ha, about 880 g/ha, about 890 g/ha, about 900 g/ha, about 910 g/ha, about 920 g/ha, about 930 g/ha, about 940 g/ha, about 950 g/ha, about 960 g/ha, about 970 g/ha, about 980 g/ha, about 990 g/ha, about 1,000, g/ha, about 1,010 g/ha, about 1,020 g/ha, about 1,030 g/ha, about 1,040 g/ha, about 1,050 g/ha, about 1,060 g/ha, about 1,070 g/ha, about 1,080 g/ha, about 1,090 g/ha, about 1,100 g/ha, about 1,110 g/ha, about 1,120 g/ha, about 1,130 g/ha, about 1,140 g/ha, about 1,150 g/ha, about 1,160 g/ha, about 1,170 g/ha, about 1,180 g/ha, about 1,190 g/ha, about 1,200 g/ha, about 1,210 g/ha, about 1,220 g/ha, about 1,230 g/ha, about 1,240 g/ha, about 1,250 g/ha, about 1,260 g/ha, about 1,270 g/ha, about 1,280 g/ha, about 1,290 g/ha, about 1,300 g/ha, about 1,310 g/ha, about 1,320 g/ha, about 1,330 g/ha, about 1,340 g/ha, about 1,350 g/ha, about360 g/ha, about 1,370 g/ha, about 1,380 g/ha, about 1,390 g/ha, about 1,400 g/ha, about 1,410 g/ha, about 1,420 g/ha, about 1,430 g/ha, about 1,440 g/ha, about 1,450 g/ha, about 1,460 g/ha, about 1,470 g/ha, about 1,480 g/ha, about 1,490 g/ha, about 1,500 g/ha, about 1,510 g/ha, about 1,520 g/ha, about 1,530 g/ha, about 1,540 g/ha, about 1,550 g/ha, about 1,560 g/ha, about 1,570 g/ha, about 1,580 g/ha, about 1,590 g/ha, about 1,600 g/ha, about 1,610 g/ha, about 1,620 g/ha, about 1,630 g/ha, about 1,640 g/ha, about 1,650 g/ha, about 1,660 g/ha, about 1,670 g/ha, about 1,680 g/ha, about 1,690 g/ha, about 1,700 g/ha, about 1,710 g/ha, about 1,720 g/ha, about 1,730 g/ha, about 1,740 g/ha, about 1,750 g/ha, about 1,760 g/ha, about 1,770 g/ha, about 1,780 g/ha, about 1,790 g/ha, about 1,800 g/ha, about 1,810 g/ha, about 1,820 g/ha, about 1,830 g/ha, about 1,840 g/ha, about 1,850 g/ha, about 1,860 g/ha, about 1,870 g/ha, about 1,880 g/ha, about 1,890 g/ha, about 1,900 g/ha, about 1,910 g/ha, about 1,920 g/ha, about 1,930 g/ha, about 1,940 g/ha, about 1,950 g/ha, about 1,960 g/ha, about 1,970 g/ha, about 1,980 g/ha, about 1,990 g/ha, or about 2,000.

The average and distribution of herbicide tolerance or resistance levels of a range of genome edited or primary plant transformation events are evaluated in the normal manner based upon plant damage, leaf chlorosis symptoms etc. at a range of different concentrations of herbicides. These data can be expressed in terms of, for example, GR50 values derived from dose/response curves having “dose” plotted on the x-axis and “percentage kill”, “herbicidal effect”, “numbers of emerging green plants” etc. plotted on the y-axis where increased GR50 values correspond to increased levels of inherent inhibitor-tolerance (e.g. increased kcat/Km value in respect of reaction with the herbicide) and/or level of expression of the expressed glucosyl transferase polypeptide.

The methods of the present invention are especially useful to protect crops from the herbicidal injury of PSII inhibitor herbicides of the classes of PSII herbicide chemistry described below and elsewhere herein. In one embodiment, suitable herbicides are selected from the group consisting of alcohols and aminals of the types described for example in patent applications CH633678, EP0297378, EP0286816, EP0334133, GB2119252, U.S. Pat. No. 4,600,430, U.S. Pat. No. 4,911,749, U.S. Pat. No. 4,857,099, U.S. Pat. No. 4,426,527, U.S. Pat. No. 4,012,223, WO2015018433, WO16162265, WO16156241, WO16128266, WO16071359, WO16071360, WO16071362, WO16071363, WO16071364, WO16071361, WO15193202, US2016318906, US2016262395, US2016251332, US2016264547, US2016200708, US2016159767, US2016159819, US2016159781, US2016168126, US2016066574 and U.S. Pat. No. 3,932,438 and U.S. Pat. No. 3,932,438 and, as for example, in structure I and structure II depicted below.

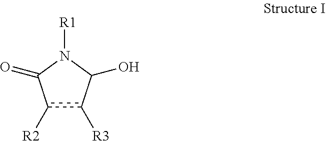

wherein R2 is halogen or C1-C3 alkoxy
and R3 is C1-C6 alkyl or C1-C3 alkoxy
and wherein R1 includes aromatic heterocycles (and partially unsaturated heterocycles), containing 1-3 nitrogens and further substituted at 1-3 positions with a broad range of substituents (H, C—C4 alkyl, t-Bu, halogen, CF3, SF5 etc.) as defined in the patent applications listed infra. Examples of aromatic headgroups R1 include substituted pyridazines, pyridines, pyrimidines, oxadiazoles, isoazoles and thiadiazoles

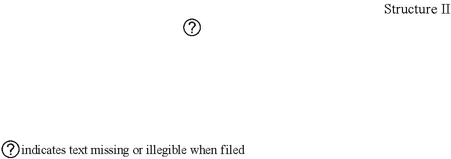

wherein R2 is C1-C6 alkyl, alkenyl, allyl, alkynyl or haloalkyl
and R3 is C1-C6 alkyl, alkoxy or allyl
and wherein R1 includes aromatic heterocycles (and partially unsaturated heterocycles), containing 1-3 nitrogens and optionally substituted at 1-3 positions with a broad range of substituents (H, C alkyl, t-Bu, halogen, CF3, SF5 etc.) as defined in the patent applications listed infra. Examples of aromatic headgroups R1 include pyridazines, pyridines, pyrimidines, oxadiazoles, isoazoles and thiadiazoles

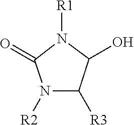

Some specific examples of these PSII herbicide chemistries are depicted infra as structures III to XII and yet further examples XIII to XXVI are depicted below.

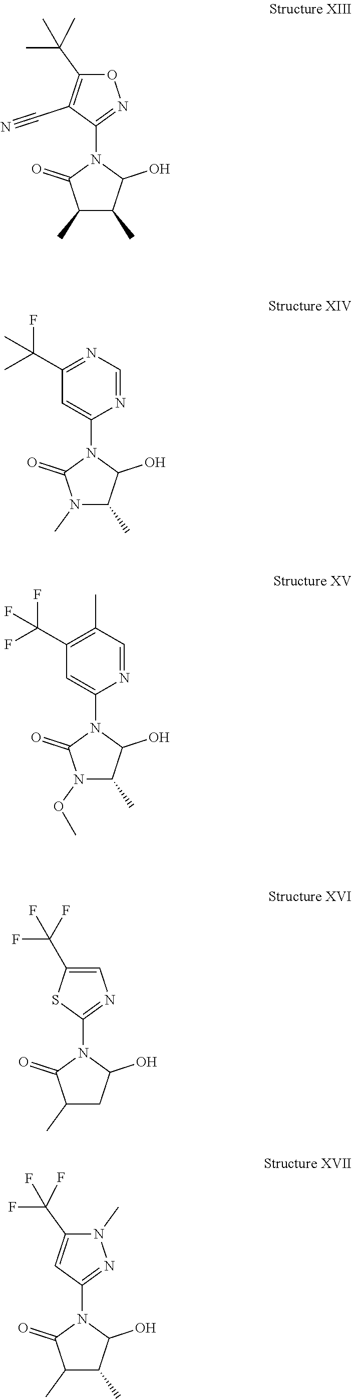 [SMILES: C[C@@H]1C(O)N(c2noc(C(C)(C)C)c2C#N)C(=O)[C@@H]1C ; C[C@H]1C(O)N(c2cc(C(C)(C)F)ncn2)C(=O)N1C ; CON1C(=O)N(c2cc(C(F)(F)F)c(C)cn2)C(O)[C@@H]1C ; CC1CC(O)N(c2ncc(C(F)(F)F)s2)C1=O ; CC1C(=O)N(c2cc(C(F)(F)F)n(C)n2)C(O)[C@@H]1C]

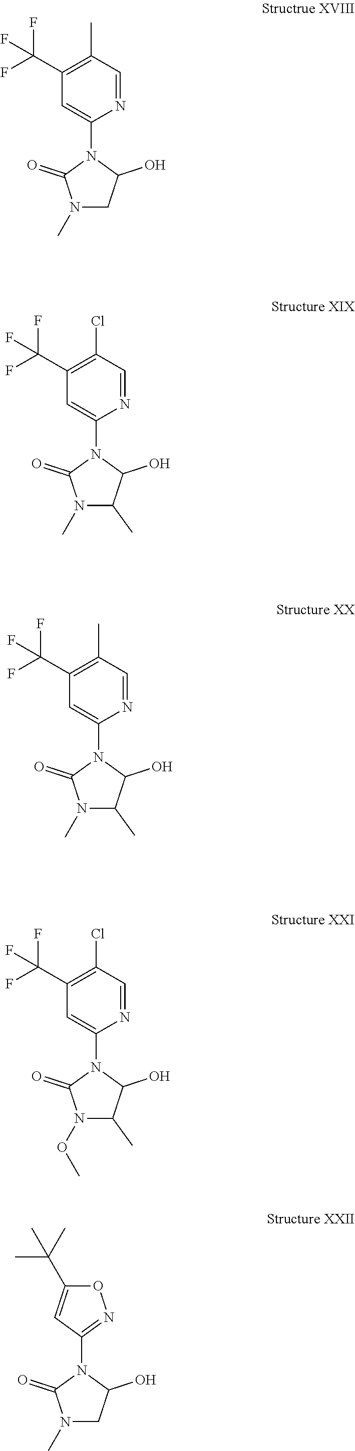 [SMILES: Cc1cnc(N2C(=O)N(C)CC2O)cc1C(F)(F)F ; CC1C(O)N(c2cc(C(F)(F)F)c(Cl)cn2)C(=O)N1C ; Cc1cnc(N2C(=O)N(C)C(C)C2O)cc1C(F)(F)F ; CON1C(=O)N(c2cc(C(F)(F)F)c(Cl)cn2)C(O)C1C ; CN1CC(O)N(c2cc(C(C)(C)C)on2)C1=O]

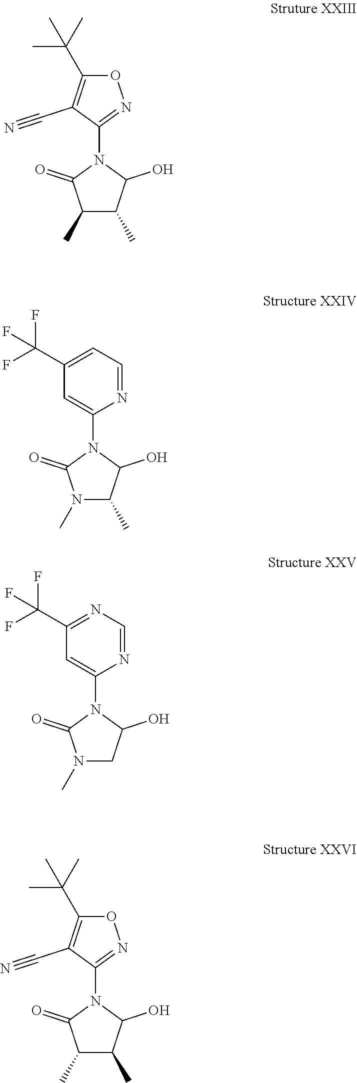 [SMILES: C[C@H]1C(=O)N(c2noc(C(C)(C)C)c2C#N)C(O)[C@@H]1C ; C[C@H]1C(O)N(c2cc(C(F)(F)F)ccn2)C(=O)N1C ; CN1CC(O)N(c2cc(C(F)(F)F)ncn2)C1=O ; C[C@@H]1C(=O)N(c2noc(C(C)(C)C)c2C#N)C(O)[C@H]1C]

The level of expression of the glucosyl transferase should be sufficient to reduce substantially (relative to likewise treated plants where the plants do not express the mutant glucosyl transferase gene) the level of parent herbicide within the cell cytoplasm within a short period of time. One of ordinary skill in the art will of course understand that certain mutant glucosyl transferase enzymes are likely to confer resistance to certain subsets of the amine, alcohol or aminal type PSII herbicides described infra and one particular enzyme may not and indeed would not be expected to provide resistance to all representatives of these classes of PSII herbicides.

### Methods of Use

The present invention further provides a method of selectively controlling weeds at a locus comprising crop plants and weeds, wherein the plants are obtained by any of the methods of the current invention described above, wherein the method comprises application to the locus of a weed controlling amount of one or more herbicides. Any of the transgenic plants described herein may be used within these methods of the invention. The term “locus” may include soil, seeds, and seedlings, as well as established vegetation. Herbicides can suitably be applied pre-emergence or post-emergence of the crop or weeds.

The term “weed controlling amount” is meant to include functionally, an amount of herbicide which is capable of affecting the growth or development of a given weed. Thus, the amount may be small enough to simply retard or suppress the growth or development of a given weed, or the amount may be large enough to irreversibly destroy a given weed.

Thus, the present invention provides a method of controlling weeds at a locus comprising applying to the locus a weed-controlling amount of one or more herbicides, where the locus comprises a transgenic plant that has been transformed with a nucleic acid molecule encoding a glucosyl transferase polypeptide or variant thereof that confers resistance or tolerance to certain amine, alcohol and aminal type herbicides, including PSII herbicides, where the said nucleic acid is present alone or in combination with one or more additional nucleic acid molecules or mutations encoding polypeptides that confer further desirable traits. In a further embodiment, there is also provided a method of controlling weeds at a locus comprising applying to the locus a weed-controlling amount of one or more herbicides, where the locus comprises a mutant plant wherein a mutant glucosyl transferase polypeptide of the current invention is expressed and the plant is thus made resistant or tolerant to the said herbicide or herbicides and where the said mutation(s) are present alone or in combination with one or more additional nucleic acid molecules and/or mutations encoding polypeptides that confer further desirable traits. In one embodiment, the further desirable trait is resistance or tolerance to an herbicide, including, for example, herbicides selected from the group consisting of amine, alcohol or aminal type PSII herbicides, HPPD herbicides, glyphosate, auxin herbicides, PPGO herbicides and glufosinate. In another embodiment, the locus comprises a transgenic plant that has been transformed with any combination of nucleic acid molecules described above, including one or more nucleic acid molecules encoding a glucosyl transferase polypeptide or variant thereof that confers resistance or tolerance to an amine, alcohol or cyclic aminal PSII herbicide in combination with at least one, at least two, at least three, or at least four additional nucleic acid molecules encoding polypeptides that confer desirable traits.

In one embodiment, the present invention provides transgenic plants and methods useful for the control of unwanted plant species in crop fields, wherein the crop plants are made resistant to certain amine, alcohol or aminal type PSII herbicides by transformation to express genes encoding glucosyl transferase polypeptides, and where an amine, alcohol or aminal PSII herbicide is applied as an over-the-top application in amounts capable of killing or impairing the growth of unwanted plant species (weed species, or, for example, carry-over or “rogue” or “volunteer” crop plants in a field of desirable crop plants). The application may be pre-or post-emergence of the crop plants or of the unwanted species, and may be combined with the application of other herbicides to which the crop is naturally tolerant, or to which it is resistant via expression of one or more other herbicide resistance transgenes. See, e.g., U.S. App. Pub. No. 2004/0058427 and PCT App. Pub. No. WO 98/20144.

In another embodiment, the invention also relates to a method of protecting crop plants from herbicidal injury. In the cultivation of crop plants, especially on a commercial scale, correct crop rotation is crucially important for yield stability (the achievement of high yields of good quality over a long period) and for the economic success of an agronomic business. Herbicide resistant or tolerant plants of the invention are also useful for planting in a locus of any short term carry-over of herbicide from a previous application (e.g., by planting a transgenic plant of the invention in the year following application of an herbicide to reduce the risk of damage from soil residues of the herbicide).

The following examples are provided by way of illustration, not by way of limitation.

### Experimental

### EXAMPLE 1

### Cloning, Expression and Assay of Zea mays BX9 and BX8 Glucosyltransferases

DNA sequences, optimized for E. coli codon usage encoding C-terminally his-tagged zmBX9 and zmBX8 polypeptides (SEQ ID No:1 and SEQ ID No: 2) derived from Zea mays are synthesized by Genewiz (South Plainfield, USA) to include 5′ NdeI and 3′ XhoI restriction sites. These are cloned into the E. coli expression plasmid pET24a (Novagen) via the NdeI and XhoI restriction sites and the resultant plasmid transformed into E. coli BL21 (DE3) and thereafter maintained with 50 μg/ml kanamycin. Transformation of E. coli BL21 (DE3) competent cells from Agilent is carried out according to the manufacturer's instructions. In brief, 100 ul aliquots of competent cells are thawed, pre-mixed on ice with 1.7 ul of β-mercaptoethanol and then incubated, swirling gently, for 30 min on ice with 1-50 ng of DNA. Each transformation reaction is briefly (45 s) warmed to 42° C. before returning to ice and then mixed with 0.9 ml of SOC medium pre-warmed to 42° C. The cell suspension is then incubated at 37° C. for 1 hour, shaking at 250 rpm before plating out 5 and 50 ul aliquots onto LB agar plates containing 50 μg/ml kanamycin. Transformed colonies are picked after an overnight grow. After pre-growth in an initial seed culture, transformed cells are transferred to Formedium Autoinduction Media (which has a Terrific broth base and includes trace elements (Cat no: AIMTB0210)) and the culture is then grown up overnight in a 1 liter flask, shaking at 20° C. Following growth approximately 10 g wet weight of cell paste is resuspended in 50 ml of lysis buffer which is 25 mM Hepes at pH 7.5 containing 25 mM Imidazole, 500 mM NaCl, and 0.5 mM TCEP (tris(2-carboxyethyl) phosphine). Cells are stirred for approximately 30 mins to resuspend and then lysed using a constant systems cell disruptor at a pressure of 20000 psi. The cell lysate is clarified by centrifugation in a Beckman JA 25.5 rotor spun for 30 mins at 25000 rpm at 4° C. Clarified lysate is then applied to a 5 ml HisTrap Crude FF column equilibrated in 25 mM Hepes buffer at pH 7.5 containing 25 mM imidazole, 500 mM NaCl and 0.5 mM TCEP. The column is washed with 20 column volumes of this buffer and bound protein is then eluted in 3.5 column volumes of 25 mM Hepes buffer at pH 7.5 containing 500 mM Imidazole, 500 mM NaCl and 0.5 mM TCEP. The eluted protein is then further purified and exchanged down a GE 26/60 5200 SEC column into 25 mM Hepes buffer at pH 7.5 containing 150 mM NaCl and 0.5 mM TCEP. 10% v/v glycerol is added to the pooled fractions prior to storage as frozen beads. Protein concentration is determined using the Nanodrop ME52070. Protein so obtained typically runs as a single major band corresponding to the expected molecular weight of ˜51 k (e.g. for C-terminally his-tagged SEQ ID NO: 1) according to SDS PAGE stained with Coomassie blue and is typically (for Zea mays bx9) judged to be >˜90% pure based on gel densitometry.

Glucosyl transferase activity is assayed via measurement of acceptor substrate-dependent production of UDP from ultrapure UDP-glucose using the Promega UDP-Glo™ method and according to the manufacturer's instructions. Assays are run in 96 well microtiter plates.

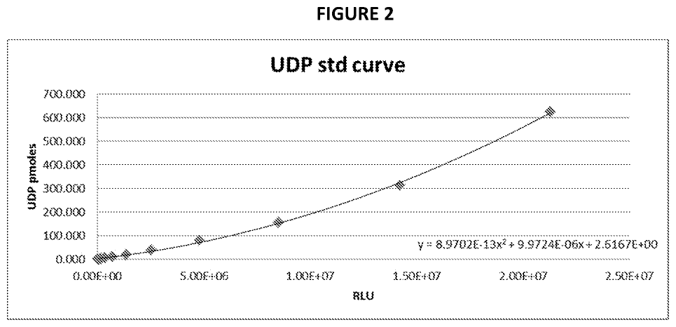

Enzyme, typically at a stock concentration of ˜2 mg/ml is diluted to an appropriate concentration in 50 mM K⁺ Hepes buffer at pH7.5 containing 0.5 mg/mL bovine serum albumin (BSA) and 5 ul aliquots of this diluted enzyme added to each well of a Perkin Elmer white ½ area 96 well plate. Assays, at 25° C., are started by addition of 20 ul of 50 mM K⁺ Hepes buffer containing 2.5 mM DTT, 0.625 mg/ml BSA, 6.25 mM Na salt of EGTA, 0.625 mM UDP-Glucose (Promega) and an appropriate concentration of test herbicide (e.g. III, IV, V, VI etc.) pre-dissolved as a stock solution at a sufficiently high concentration in dimethylsulfoxide (DMSO) that the final concentration of DMSO does not exceed more than about 0.75% v/v DMSO in the final assay reaction. Assays are run for an appropriate time (usually 10 to 60 min) so that the amount of UDP formed lies within the most nearly linear part of the UDP standard curve and are stopped with the addition and mixing of 25 ul UDP-Glo™ detection reagent (prepared as described below). Plates are then incubated at room temperature for 60 minutes and then read in a luminescence plate reader (Perkin Elmer Envision 2130 Multilabel Reader). A UDP standard curve is run alongside each set of assays and reagent blank control assays run with DMSO in place of test herbicide. Typically, 300 pmol of UDP corresponds to a Relative Luminescence Unit (RLU) reading of about 1.5E7 and the response curve between 0 and 600 pmol is fitted to a polynomial function (see for example FIG. 2).

The UDP-Glo™ reagents are made up and used according to the manufacturer's instructions. Thus, Nucleotide Detection Buffer and ATP are combined to make nucleotide detection reagent (NDR) dispensed into aliquots and frozen to be freshly thawed before use. UDP-Glo™ working solution is prepared by diluting UDP-Glo™ high concentrate 75 fold into 50 mM Hepes buffer at pH7.5 and then UDP-Glo™ detection reagent is freshly prepared as a 100 fold dilution of UDP-Glo™ working solution into NDR.

Km and kcat values in respect of test herbicides are obtained by carrying out experiments to measure initial rates over a suitable range of concentrations of acceptor herbicide substrate at a fixed, near saturating concentration of UDP-Glucose (usually 0.5 mM). Km and kcat values in respect of UDP-glucose are derived by carrying out experiments to measure initial rates over a suitable range of concentrations of UDP glucose out at a fixed near saturating concentration of acceptor substrate. Best fit values of kcat, Km and kcat/Km are obtained by direct fitting of the data to the Michaelis-Menten equation using Graphpad Prism™ software.

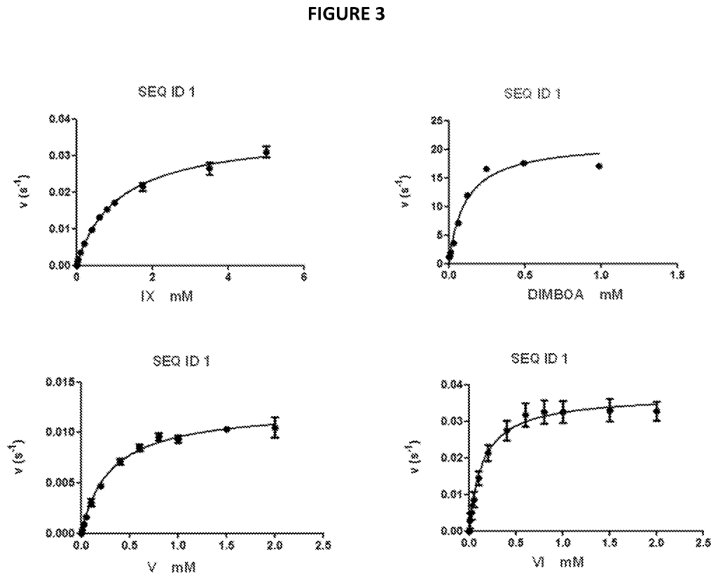

Some results obtained using the assay are depicted in FIG. 3 and are summarized in Table 10.

TABLE 10
Estimates of kinetic parameters for Zea mays bx9
(C-terminally his tagged SEQ ID NO: 1) assayed with
DIMBOA and herbicides V, VI and IX as acceptor substrates.
Estimates of Km and kcat of 2,4-dihydroxy-7-methoxy-
1,4-benzoxazin-3-one (DIMBOA) and of various
herbicides in respect of the C-terminally his tagged
polypeptide of SEQ ID No: 1.

| Acceptor | Km | std. | kcat/Km | std. | kcat | std. |
|---|---|---|---|---|---|---|
| substrate | (mM) | error | (/s/mM) | error | (/s) | error |
| V | 0.3 | 0.02 | 0.041 | 0.002 | 0.012 | 0.001 |
| | 0.314 | 0.022 | 0.066 | 0.003 | 0.021 | 0.001 |
| VI | 0.146 | 0.016 | 0.254 | 0.023 | 0.037 | 0.01 |
| | 0.147 | 0.016 | 0.299 | 0.025 | 0.044 | 0.002 |
| IX | 1.098 | 0.065 | 0.033 | 0.001 | 0.036 | 0.001 |
| DIMBOA | 0.133 | 0.022 | 156.6 | 18.8 | 20.82 | 1.248 |

In an alternative method for assaying the various enzymes, the test glucosyl transferase enzyme is reacted with substrates exactly as above except that the assay is stopped by adding an equal volume of acetonitrile. Alternatively 50 or 100 μl samples from assay reactions are added to 500 μl ethyl acetate to stop the reaction. In this case samples are then vortexed and 400 μl of the ethyl acetate partition removed, dried down, and resuspended in 100 μl 80:20 acetonitrile/water. The formation of product gluco side and/or disappearance of substrate test herbicide is then monitored directly by LC/MS. Samples are analyzed by LC-MS using an Agilent 1290 liquid chromatography system and Thermo Q-Exactive mass spectrometer. The chromatography is achieved on a Waters Acquity C18 BEH (50×2.1 mm) 1.7 μm particle size column, using a 6 minute gradient run of Water (0.2% formic acid) and Acetonitrile. The Q-Exactive is operated in positive ionisation electrospray mode, using Full scan-AIF mode, at 35,000 resolution, between 100-800 m/z. All analytes are identified from the full scan data to within at least 5 ppm accuracy of their predicted pseudo-molecular ion [M+H]⁺ m/z value. In order to obtain quantitative data standard curves are run using herbicides and herbicide glucosides synthesized as standards. Where these synthetic glucosides were not available the LC/MS assay could only be used to provide relative data.

### EXAMPLE 2

### Cloning, Expression and Assay of Variant Sequences of the Zea mays BX9 Glucosyltransferase Gene

The w/t zmBX9 glucosyltransferase polypeptide sequence (SEQ ID NO: 1) is used as the base sequence to create and screen for mutants exhibiting greater activity than the w/t sequence towards herbicide example V. The amino acid positions listed in table 11 are selected for a saturation mutagenesis approach (i.e. replacing the amino acid of interest with every other amino acid alternative which therefore leads to 19 variants per amino acid position investigated).

Assay methods are similar to those described in example 1 except that in this case, because of the high numbers to test, assays are carried out upon extracts (rather than purified proteins) of cells grown and induced for expression in deep well plates. Thus saturation libraries of DNA sequences encoding mutants of zmBX9 derived from Zea mays, optimized for E. coli codon usage, are synthesized with a C-terminal 6×His purification tag and cloned into the E. coli expression plasmid pET24a (Novagen) via the NdeI and XhoI restriction sites.

Competent BL21 (DE3) cells are transformed as in the foregoing examples and, following seed culturing, grown and autoinduced in plates in a 0.5-1 ml volume of autoinduction medium containing 50 ug/ml kanamycin. Plates are incubated at 37° C. and shaken at 900 rpm. Growth is monitored by taking 20 ul aliquots, diluting 10 fold into flat-bottomed 96 well microtiter plates and reading OD₆₀₀ₙ at t=0, 1, 2, 2½ and 3 hours if necessary. At a (corrected) OD_{600 nm}˜0.2, plates are transferred to 20° C. and shaken overnight at 900 rpm in a Jencons VWR plate incubator. After ˜18-20 h the final OD₆₀₀ readings are recorded and the plates centrifuged at 4600 rpm (bench top centrifuge) for 10 min at 4° C. The supernatant is discarded and the pellets then washed with 0.25 ml PBS by repeat centrifugation and removal of supernatant before freezing cell pellets at −80° C. To prepare extracts, plates are allowed to thaw for 30 min and then pellets are resuspended in 0.25 ml of a suitable lysis buffer (for example—50 mM Tris HCL buffer at pH 8.0, 5% glycerol containing 50 mM NaCl and lysonase) mixed, incubated at room temperature for a sufficient time for lysis to be near complete and then centrifuged to remove cell debris and 100 ul of supernatant extract removed to a 96 well V-shaped well plate and stored on ice prior to assay.

Protein determination of extracts using the Bradford method is used to verify that protein concentrations across the his-tagged mutant and his-tagged w/t Zea mays bx9 expressing E. coli extracts are consistent. They usually were to within about 10% thus confirming that cell growth and lysis was generally consistent across the plate. Similarly, Coomassie dye-stained SDS PAGE confirmed that the majority of (but not all) single mutants of bx9 polypeptide were expressed to about the same consistent high level (estimated to be about 40% of the total soluble protein) similar to the level of expression seen with the unmutated w/t protein. UDP—luminescence assays of the well-grown extracts are carried out as described in Example 1 with test herbicide at a fixed concentration (typically 0.25, 0.5 or 1 mM) and UDP-glucose at 0.5 mM. Optionally plate assays are stopped with brief heating to 95 C before returning to ice and addition of UDP-Glo detection reagent followed by incubation at lab temperature. Each plate test of BX9 mutant extract is run at a suitable dilution to maximize signal to background and includes at least triplicated control wells containing 1) w/t bx9 extract and 2) extract from an E. coli line expressing an H24A mutant form of bx9 which is catalytically inactive which, in this example, is used as the blank control. In addition a UDP standard curve is run alongside each set of plate tests.

Data from such tests compared the activity of each of 19 mutations at various positions in the polypeptide sequence of bx9 with the activity of the wild type. The activity observed with w/t bx9 (the signal observed from w/t bx9 minus the control background signal from the H24A mutant bx9 mutant) on each plate was defined as a value of 1.0. The activity of the various test mutant extracts on the same plate (the signal observed from the test mutant bx9 extract minus the control background signal from the H24A mutant bx9 mutant) was then expressed as a fraction of the level of the activity of the w/t and thus the ‘improvement factor’ expressed as a decimal where, for example, ‘0.5’ means half the activity of the wild type and 2.0 means twice the activity of the wild type bx9. Optionally the improvement factors are further normalized to allow for any measured differences in the protein concentrations of the extracts although generally growth and lysis are seen to be consistent and the effect of such additional normalization minor. However, on occasion, particular single mutations resulted in significantly decreased expression of the mutant bx9 protein. Thus, in a further extension of the method, the concentration of expressed bx protein in each individual well extract was measured using a highly specific ELISA assay based upon antibodies raised to C-terminal His tagged Zea mays bx9 protein purified as described in the foregoing example.

For ELISA assay development, the immunizing agent was the C terminally his-tagged SEQ ID No: 1 polypeptide that was purified from an E. coli expression system as described in example 1. After the initial immunizing injection, the rabbit or goat is boosted after 21 days and thereafter every 21 days. Serum is taken 7 and 14 days after the final boost. The immunoassay used is a quantitative sandwich assay employing two Zea mays bx9-raised polyclonal antibodies purified using Protein A (PA) or Protein G (PG). High-binding polystyrene plates (Nunc Maxisorp #430341) are coated at 4° C. overnight with 10 μg/ml goat anti-BX9 PG in 25 mM borate, 75 mM NaCl, pH 8.5 and washed five times with Phosphate Buffered Saline (PBS)+0.05% Tween-20. Samples and standards (160, 80, 40, 20, 10, 5, 2.5, and 0 ng/ml of purified C terminally his tagged SEQ ID No: 1 protein) are prepared in ELISA diluent (PBS containing 1% BSA, 0.05% Tween-20). One hundred microliters of each appropriately diluted sample or standard is added to the wells of a plate, incubated for 1 hr. at ambient temperature with shaking at 200 rpm, and washed five times. Rabbit anti-BX9 PA (100 μl/well) at 1 μg/ml is then added to the plate, incubated for 1 hr. at ambient temperature with shaking at 200 rpm, and washed as before. Donkey anti-rabbit conjugated to alkaline phosphatase (Jackson ImmunoResearch, West Grove, Pa.) at 1 μg/ml is added to the plate (100 μl/well), incubated at ambient temperature with shaking at 200 rpm, and washed. Substrate p-nitrophenyl phosphate (Surmodics) is added and allowed to develop for 30 min at ambient temperature. The absorbance is measured at 405 nm using a microplate reader (BioTek Powerwave XS2, Winooski, Vt.). The standard curve used a four-parameter curve fit to plot the concentrations of Zea mays bx9-derived protein versus the absorbance. Specifications are determined by calculating the 2 SD range of the absorbances for each standard from 25 assays. Most assays should fall within the 2 SD range and quantitation from assays that fall within the 2 SD range are acceptable. Assay precision was within 20% for samples falling within the linear portion of the standard curve

Making use of the ELISA assay it is then possible to calculate a specific activity (e.g. in Relative Luminescence Units (RLU)/min/ug bx protein) based upon the amount of each mutant bx9 polypeptide expressed in each well. The thus obtained specific activity data are again normalized versus the average specific activity observed with w/t Zea mays bx9 extract on the same plate with this control value set as 1.0. The specific activity of the various test mutant extracts on the same plate are then expressed as a fraction of the specific activity of the w/t bx9 and thus the ‘improvement factor’ versus the w/t expressed as a decimal where, for example, ‘0.5’ means half the specific activity of the wild type and 2.0 means twice the specific activity of the wild type. However, as will be readily apparent to the skilled man, particularly in cases where the UDP assay signal was low (with poor signal to noise versus the background) and/or the ELISA measurement of bx protein also low such a method is prone to generate spurious high specific activity numbers and uncertain numbers. Accordingly some results were too uncertain to include or should only be taken useful to set approximate lower bounds on the improvement factor in specific activity versus w/t bx9.

Thus, Table 11 provides preferred amino acid changes selected on the basis of their estimated improvement factors at various sequence positions relative to the C-terminally his-tagged Zea Mays bx9 polypeptide SEQ. ID NO 1. Preferred or neutral amino acid substitutions (giving approximately neutral improvement factors in the range 0.75-1.25) and most preferred amino acid substitutions giving improvement factors>1.5) are tabulated in separate columns according to whether they were selected on an activity basis only (Relative Luminescence Units per minute per ul of extract) or a specific activity basis (Relative Luminescence Units per minute per ug of bx protein). The differences between the two bases for selection is that the former also selects for amino acid changes that are better expressed in E. coli and where this appears typically to also translate to improved expression in a plant cell and where improved expression, along with improved specific activity, is also a desirable characteristic to select for conferring herbicide tolerance.

TABLE 11
Preferred and most preferred amino acid substitutions at a range of positions
within the polypeptide sequence of SEQ ID No: 1.
Numbers following single letter code amino acid lists are measured improvement factors (′IF′)
rounded to the nearest 0.5 relative to the wit sequence either based on measured extract activities
per ml or per ug of protein (i.e. specific activities based on ELISA-detected amounts of bx
protein). All activities were measured with compound V as acceptor substrate. Blank lines
where no data have been added indicates that all variants at the corresponding amino acid
position were significantly less active than the w/t amino acid (i.e. no equivalent or beneficial
mutants were detected).

| Zm | | | | | |
|---|---|---|---|---|---|
| BX9 | | | | | Most Preferred |
| amino | Amino acid | | | Preferred amino | amino acid |
| acid | context | | | acid | substitutions |
| position | (Amino acid | | Most Preferred amino | substitutions | and (IF) based |
| (SEQ | of | Preferred amino acid | acid substitutions | and (IF) based on | on specific |
| ID No | interest | substitutions and (IF) | and (IF) based on | specific activity | activity RLU/ |
| 1) | underlined) | based on RLU/min/ul | RLU/min/ul | RLU/min/ug | min/ug |
| F19 | VFPFPFQ | M(1.0) | | M(1.0) | |
| F21 | PFPFQGH | | Y(2.0) | | Y(2.0) |
| Q22 | FPFQGHF | H,P,M(1.0) | | C,I(1.0) | H,M(2.0) |
| G23 | PFQGHFN | | | | |
| E76 | LASEDIA | M(1.0) | | LI(1.0) | |
| D77 | ASEDIAA | | | | |
| I78 | SEDIAAI | | | | F,Y(2.0) |
| A79 | EDIAAIV | S,N,Q(1.0); G,E(1.5) | | T,C,P,W,Y(1.0); | G,E,M(2.0) |
| | | | | F,L,H,Q,N,S(1.5) | |
| I81 | IAAIVTT | | | W,C(1.0); | |
| | | | | V(1.5) | |
| V82 | AAIVTTL | | | C,P(1.0); A(1.5) | |
| L85 | VTTLNAS | | | | |
| N86 | TTLNASC | | | D(1.5) | |
| V116 | FTDVSWN | L,I(1.0) | | I(1.0) | |
| S117 | TDVSWNA | | | C(1.5) | T,V,I(2.0) |
| W118 | DVSWNAV | | | F(1.0) | Y(2.0) |
| M135 | ALGMMTA | T,C,L(1.0) | | I(1.0); L(1.5) | H(2.0); S(2.5) |
| | | | | | A(3.0); T(4.0) |
| M136 | LGMMTAS | | | P(1.0) | |
| T137 | GMMTASA | | | | |
| A138 | MMTASAA | | | S(1.5) | |
| S142 | SAASLRD | | | | |
| L143 | AASLRDY | M,Y(1.0) | | Y(1.0); K(1.5) | |
| D145 | SLRDYMA | | | | |
| Y146 | LRDYMAY | | | | |
| I153 | TLIDKGY | T,C,K,V,L,M,Y(1.0); | | S,M,W(1.0); | R(2.5); F(3.0) |
| | | Q,R,F(1.5) | | T,Q,K,H,V,L(1.5) | |
| L181 | KDLLRVD | | | C,I,M(1.5) | |
| V183 | LLRVDTS | | | L,I(1.0) | |
| F191 | LEEFAEL | | | S,V,W(1.0); | |
| | | | | T,I,M,L(1.5) | |
| L194 | FAELLAR | G,E,D(1.0); T,Q(1.5) | I(2.0); A(2.5); | G,Q(1.0); | I,V,A(2.5); |
| | | | C,N,V(3.0) | T,D(1.5) | N(3.0); C(3.5) |
| L195 | AELLART | I(1.0) | | I(1.5) | |
| T198 | LARTVTA | | V(2.0) | | V(2.0) |
| V199 | ARTVTAA | M,N(1.0) | | H,Y(1.0) | |
| A202 | VTAARRA | S,I,C,M(1.0) | | T,V,I(1.0) | F,Y(2.0) |
| | | T,I,V,L,F(1.5) | | N,L(1.5) | |
| F210 | GLIFNTF | M,W(1.0) | | W(1.5) | |
| T220 | ETDTLAE | G,Q,D,V(1.0) | S,R(2.5) | A(1.0); | R(2.0); |
| | | C,A,N,E,L,I,M(1.5) | K(3.0); F,W,Y(3.5); | G,C,N,D,E,V,L, | M(2.5) |
| | | | H,P(4.0) | I(1.5) | K,H(3.0); Y(3.5) |
| | | | | | P(4.0); F,W(7.5) |
| M279 | FGSMAAM | | | V,I,W(1.0) | F(2.5) |
| A280 | GSMAAMD | V(1.0) | | V(1.0) | |
| A281 | SMAAMDP | S,T,V(1.0) | C,Q(3.0) | S,T,V(1.0) | C,Q(2.0) |
| | | L(1.5) | M,R(5.0); K(6.0) | L(1.5) | R(3.5); M(4.0) |
| | | | | | K(5.0) |
| A334 | IVVAWAP | N(1.0) | K,R(2.0) | N(1.0) | K,R(2.0) |
| | | S,Q,V,L(1.5) | | S,Q,V,L(1.5) | |
| I363 | VEAISEG | C(1.0) | A,V(2.0) | C,V(1.0) | A,F(2.0); |
| | | S,T,F,W(1.5) | L,M(3.0) | T(1.5) | L(2.5) |
| | | | | | S(5.0); Q,W |
| | | | | | (>10.0) |
| V370 | VPMVCCP | T,C,L,M(1.0) | | C,A,L,I,M(1.0) | Y(3.0); |
| | | A,I(1.5) | | G,S,T,F(1.5) | N(4.0) |
| C372 | MVCCPRH | I(1.0) | | I(1.0) | |
| G376 | PRHGDQF | C,M(1.0) | L(2.0) | | |
| N381 | QFGNMRY | | | | |
| A432 | KIAAAKG | | L,H,Q,T,F,Y(>5.0) | | D,H,F,P,E,R, |
| | | | | | N,K,V(>10.0) |

### EXAMPLE 3

### Cloning, Expression, Purification and Assay of Various Mutants and Combinations of Mutants of the (C-Terminally His Tagged) Zea mays bx9 Polypeptide

Variants of C-terminally his tagged SEQ ID No:1 (w/t bx9 from Zea mays), exemplified as SEQ ID 16-30, were cloned, purified and assayed as described in Example 1. FIG. 4 depicts the data from some experiments to determine the kinetic parameters of some of these variants in respect of herbicide VI and table 12 summarizes the estimates of kinetic parameters obtained from further such experiments in respect of a range of herbicides.

TABLE 12
Estimated kinetic parameters of the w/t and of various
mutants of Zea mays bx9 glucosyl transferase assayed
versus a range of herbicides
(polypeptides were the C-terminally his-tagged
derivatives of the polypeptide sequence IDs
listed in the table)

| Polypeptide | | Km | std | kcat/Km | std | kcat | std |
|---|---|---|---|---|---|---|---|
| SEQ | STRUCTURE | (mM | error | (/s/mM) | error | (/s) | error |
| SEQ ID 1 | VI* | 0.1117 | 0.0178 | 0.3836 | 0.0444 | 0.0430 | 0.0020 |
| SEQ ID 1 | VI | 0.1470 | 0.0160 | 0.2990 | 0.0250 | 0.0440 | 0.0020 |
| SEQ ID 1 | XXI | 0.0020 | 0.0040 | 0.0620 | 0.1200 | 0.0001 | 0.0000 |
| SEQ ID 1 | XVIII | 2.5000 | 1.6560 | 0.0020 | 0.0000 | 0.0040 | 0.0020 |
| SEQ ID 1 | V* | 0.0923 | 0.0182 | 0.2028 | 0.0359 | 0.0181 | 0.0008 |
| SEQ ID 1 | V | 0.3140 | 0.0220 | 0.0660 | 0.0030 | 0.0210 | 0.0010 |
| SEQ ID 17 | VI | 0.0022 | 0.0005 | 938.05~ | 198.0150 | 2.0805 | 0.0694 |
| SEQ ID 17 | XI* | 10.5300 | 7.4230 | 0.1900 | 0.0100 | 2.0400 | 1.3400 |
| SEQ ID 17 | XXI* | 0.1900 | 0.0270 | 11.2500 | 1.1830 | 2.0800 | 0.1000 |
| SEQ ID 17 | XVIII* | 0.8000 | 0.0940 | 0.4800 | 0.0260 | 0.3800 | 0.0250 |
| SEQ ID 17 | V | 0.1097 | 0.0078 | 7.7965 | 0.4357 | 0.8518 | 0.0146 |
| SEQ ID 24 | V | 0.0455 | 0.0014 | 57.5000 | 1.6390 | 2.6140 | 0.0151 |
| SEQ ID 21 | V | 0.0573 | 0.0026 | 32.4600 | 1.2490 | 1.8590 | 0.0209 |
| SEQ ID 22 | XI* | 0.3200 | 0.0350 | 2.5000 | 0.1760 | 0.8000 | 0.0350 |
| SEQ ID 22 | XXI* | 0.0200 | 0.0040 | 68.8800 | 9.5520 | 1.6800 | 0.0550 |
| SEQ ID 22 | XVIII* | 0.2000 | 0.0210 | 5.9500 | 0.4620 | 1.1600 | 0.0420 |
| SEQ ID 22 | V | 0.0147 | 0.0009 | 89.6850 | 4.7365 | 1.3215 | 0.0150 |
| SEQ ID 20 | V | 0.0069 | 0.0009 | 287.9500 | 33.6300 | 1.9995 | 0.0438 |
| SEQ ID 20 | XI* | 0.2300 | 0.0160 | 10.5000 | 0.5360 | 2.3700 | 0.0610 |
| SEQ ID 20 | XXI* | 0.0200 | 0.0050 | 78.5300 | 14.1100 | 1.8800 | 0.0800 |
| SEQ ID 20 | XVIII* | 0.1900 | 0.0250 | 11.2700 | 1.0880 | 2.1500 | 0.0960 |
| SEQ ID 25 | VI | 0.0085 | 0.0005 | 380.1000 | 20.2700 | 3.2400 | 0.0385 |
| SEQ ID 16 | VI | 0.0028 | 0.0002 | 880.3~ | 49.0500 | 2.4480 | 0.0252 |

~In these cases Km was too low to be determined accurately and the corresponding estimates of kcat/Km are suspect and likely too high
*The asterisk denotes where kcat and Km estimates were calculated subtracting a control value from the inactive H24A mutant of bx9 rather than by using DMSO reagent blanks as control. This generally led to slightly higher estimates of kcat/Km than in example 1. With such low values of activity as observed with, for example, the w/t bx9 sequence there is a wider range of uncertainty in calculations of kcat and Km because the low background rate of uncoupled UDP glucose hydrolysis (which is somewhat stimulated by DMSO) becomes a larger part of the total signal. This background rate is not significant when activities are high but creates more uncertainty in the parameters derived from measurements at low levels of activity and with high Km substrates. Thus, for example, for glucosylation of structure V catalyzed by C terminally his tagged SEQ ID NO: 1, the true value of kcat/Km will lie somewhere in between ~0.07 and 0.2 corresponding to the two limiting assumptions underpinning the adoption of one or other control that either a) addition of herbicide substrate completely displaces and inhibits uncoupled UDP-glucose hydrolysis or that b) addition of substrate has no suppressive effect at all on this background rate. The two alternative blank subtractions effectively set lower and upper bounds on the kinetic values. This ambiguity can, in principle, be resolved by using the LC/MS rather than UDP-based luminetric assays.

Table 13 summarizes the results obtained from assays, run as described in Example 1, using a variety of alcohol and aminal PSII herbicides as substrates of the Zea mays bx9 w/t (i.e. C-terminally his tagged SEQ ID no 1) and various mutants of the same, selected from SEQ ID NO: 16-30). All of these proteins were C-terminally his-tagged, expressed and purified as described in Example 1. Assays were run and analyzed as described in example 1. It is seen that various of the mutations herein and combinations thereof led to significant improvements (over the w/t bx9 protein) in catalytic activity versus various of the herbicides and that these improvements are often of sufficient magnitude to be useful for conferring improved herbicide tolerance upon crop. This is especially the case given that even the unmutated w/t bx9 enzyme which has only modest glucosyl transferase activity (for example against compounds V and VI) was nevertheless adequately active to confer significant herbicide tolerance in the glass house (as shown in example 9) even without mutational improvement. It is also seen that particular mutations can lead to very much improved activity in respect of some chemistries but not to others but that, overall, there is at least one variant within the scope of the current invention that provides significant and useful improvement in tolerance to each of the chemistries tested.

TABLE 13
Activities with various alcohol and aminal herbicides tested as substrates of w/t
and mutant forms of Zea mays bx9 glucosyl transferase.

| | | SEQ ID NO 1 | | SEQ ID NO 17 | | SEQ ID NO 18 | | SEQ ID NO 19 | |
|---|---|---|---|---|---|---|---|---|---|
| | | Activity | | Activity | | Activity | | Activity | |
| | Herbicide | (pmol/ | | (pmol/ | | (pmol/ | | (pmol/ | |
| | concen- | sec/ | | sec/ | | sec/ | | sec/ | |
| | tration | pmol | | pmol | | pmol | | pmol | |
| Compound | (mM) | enzyme) | st. error | enzyme) | st. error | enzyme) | st. error | enzyme) | st. error |
| V | 0.300 | 0.005 | 0.000 | 0.565 | 0.066 | 1.582 | 0.093 | 1.651 | 0.009 |
| VI | 0.250 | 0.012 | 0.000 | 1.003 | 0.120 | | | | |
| IX | 1.000 | 0.017 | 0.000 | 0.035 | 0.000 | 0.115 | 0.011 | 0.070 | 0.004 |
| XIII | 1.000 | −0.001 | 0.001 | 0.020 | 0.001 | 0.051 | 0.031 | 0.095 | 0.007 |
| IV | 0.200 | 0.000 | 0.000 | 0.019 | 0.000 | 0.145 | 0.009 | 0.053 | 0.004 |
| XXVI | 1.000 | −0.001 | 0.001 | 0.020 | 0.001 | 0.051 | 0.031 | 0.095 | 0.007 |
| III | 1.000 | 0.001 | 0.000 | 0.032 | 0.001 | 0.170 | 0.026 | 0.097 | 0.007 |
| XXV | 1.000 | 0.000 | 0.000 | 0.106 | 0.002 | 0.553 | 0.026 | 0.581 | 0.004 |
| XXIV | 1.000 | 0.001 | 0.001 | 0.528 | 0.033 | 0.982 | 0.020 | 1.358 | 0.052 |
| XXIII | 1.000 | −0.003 | 0.000 | 0.014 | 0.000 | 0.089 | 0.002 | 0.072 | 0.008 |
| VIII | 0.200 | −0.002 | 0.000 | 0.008 | 0.001 | 0.124 | 0.008 | 0.077 | 0.013 |
| XXII | 1.000 | 0.000 | 0.000 | 0.023 | 0.001 | 0.186 | 0.005 | 0.080 | 0.005 |
| XI | 0.200 | 0.000 | 0.000 | 0.120 | 0.002 | 0.710 | 0.030 | 0.898 | 0.021 |
| XIX | 1.000 | −0.001 | 0.000 | 0.157 | 0.024 | 0.467 | 0.013 | 0.363 | 0.008 |
| XXI | 0.200 | 0.000 | 0.000 | 0.919 | 0.011 | 1.524 | 0.012 | 1.534 | 0.010 |
| XX | 0.200 | −0.001 | 0.000 | 0.244 | 0.002 | 0.751 | 0.003 | 0.662 | 0.012 |
| XII | 1.000 | 0.007 | 0.001 | 0.018 | 0.002 | | | | |
| XVII | 1.000 | −0.001 | 0.000 | 0.083 | 0.016 | 0.237 | 0.064 | 0.331 | 0.042 |
| XVIII | 0.200 | 0.001 | 0.000 | 0.231 | 0.018 | 0.898 | 0.100 | 0.964 | 0.043 |
| X | 1.000 | −0.001 | 0.000 | | | | | 0.102 | 0.008 |
| XVI | 0.200 | | | 0.073 | 0.001 | 0.359 | 0.000 | | |
| XV | 0.200 | | | 0.833 | 0.006 | | | 1.391 | 0.030 |
| XIV | 0.200 | −0.001 | 0.000 | 0.037 | 0.001 | | | | |
| VII | 0.210 | 0.000 | 0.000 | | | 0.087 | 0.002 | | |
| | | SEQ ID NO 20 | | SEQ ID NO 21 | | SEQ ID NO 22 | | SEQ ID NO 23 | |
| | | Activity | | Activity | | Activity | | Activity | |
| | | (pmol/ | | (pmol/ | | (pmol/ | | (pmol/ | |
| | | sec/ | | sec/ | | sec/ | | sec/ | |
| | | pmol | | pmol | | pmol | | pmol | |
| | Compound | enzyme) | st. error | enzyme) | st. error | enzyme) | st. error | enzyme) | st. error |
| | V | 1.889 | 0.290 | 1.548 | 0.020 | 1.221 | 0.054 | 1.246 | 0.008 |
| | VI | | | | | | | | |
| | IX | 0.068 | 0.006 | 0.012 | 0.001 | 0.071 | 0.003 | 0.013 | 0.001 |
| | XIII | 0.143 | 0.011 | 0.040 | 0.004 | 0.073 | 0.010 | 0.034 | 0.001 |
| | IV | 0.079 | 0.008 | 0.008 | 0.000 | 0.099 | 0.001 | 0.023 | 0.000 |
| | XXVI | 0.143 | 0.011 | 0.040 | 0.004 | 0.073 | 0.010 | 0.034 | 0.001 |
| | III | 0.144 | 0.015 | 0.020 | 0.002 | 0.144 | 0.003 | 0.066 | 0.003 |
| | XXV | 0.758 | 0.007 | 0.170 | 0.001 | 0.458 | 0.007 | 0.449 | 0.011 |
| | XXIV | 1.462 | 0.023 | 1.154 | 0.129 | 0.908 | 0.064 | 0.816 | 0.040 |
| | XXIII | 0.119 | 0.001 | 0.034 | 0.000 | 0.067 | 0.003 | 0.021 | 0.001 |
| | VIII | 0.145 | 0.011 | 0.016 | 0.002 | 0.061 | 0.004 | 0.017 | 0.003 |
| | XXII | 0.120 | 0.003 | 0.015 | 0.001 | 0.101 | 0.003 | 0.020 | 0.000 |
| | XI | 1.046 | 0.038 | 0.374 | 0.003 | 0.548 | 0.006 | 0.227 | 0.010 |
| | XIX | 0.414 | 0.011 | 0.234 | 0.010 | 0.315 | 0.020 | 0.207 | 0.008 |
| | XXI | 1.457 | 0.010 | 1.827 | 0.017 | 1.714 | 0.016 | 1.746 | 0.014 |
| | XX | 0.698 | 0.012 | 0.338 | 0.010 | 0.563 | 0.007 | 0.249 | 0.002 |
| | XII | | | | | | | | |
| | XVII | 0.402 | 0.032 | 0.195 | 0.015 | 0.134 | 0.009 | 0.096 | 0.002 |
| | XVIII | 1.141 | 0.018 | 0.425 | 0.040 | 0.933 | 0.031 | 0.253 | 0.000 |
| | X | 0.137 | 0.018 | | | | | | |
| | XVI | 0.687 | 0.038 | | | 0.285 | 0.013 | | |
| | XV | | | 1.711 | 0.002 | 1.530 | 0.008 | 1.641 | 0.019 |
| | XIV | 0.411 | 0.010 | | | | | 0.196 | 0.000 |
| | VII | 0.049 | 0.003 | | | 0.042 | 0.002 | | |

### EXAMPLE 4

### Cloning, Expression and Assay of Various Mutant and Hybrid Sequences of Zea mays BX8 Glucosyltransferase

C and N-terminally his tagged zmBX8 (SEQ ID No:2) polypeptides were cloned for expression in E. coli as described in example 1 so as to produce both C and N terminal his tagged versions of the protein . Mutant versions of the C-terminally his-tagged BX8 gene are similarly obtained and expressed in E. coli BL21 DE3 so as to produce V367I, H376C (SEQ ID NO:38); I374V, H376C (SEQ ID NO:40); V367I, I374V (SEQ ID NO:39); E256V, R265Q (SEQ ID NO:43) and A248T, E256V (SEQ ID NO:42) double mutant polypeptides as well as a D170E, A72P, A174P(SEQ ID NO:41) triple mutant bx8 derived polypeptide. In addition, short regions (up to ˜20 amino acids) of the zmBX9 coding sequence are introduced into the zmBX8 sequence to produce a series of hybrid polypeptide sequences. In total 11 such hybrid polypeptides were designed and these are listed as SEQ IDs 49, 50, 51, 52, 53, 54, 55, 56, 57, 58 and 59. DNA sequences encoding these hybrid polypeptide sequences were synthesized by Genewiz (South Plainfield, USA) as E. coli optimized sequences and cloned into pET24a (Novagen) using NdeI and XhoI. All of the hybrid sequences were designed with a C-terminal 6×His purification tag. Expression and purification was carried in E. coli BL21 (DE3) with 50 μg/ml kanamycin as described in example 1.

The glucosyl transferase activities of the mutant bx8-derived polypeptides expressed in these strains were initially assayed versus herbicide V as described in Example 1. The partly purified C-terminally his tagged w/t bx8 enzyme (SEQ ID No: 2) catalyzed glucosyl transfer to herbicide V at a low rate estimated to be less than about 0.002/s at 1 mM herbicide V which, because of a relatively high background of UDP formation in the DMSO (no substrate reagent control) was difficult to quantify using the luminescence assay. Thus further assays were run by LC/MS using which technique the formation of an O-beta glucosyl glucoside of herbicide V could more easily be confirmed but only quantified in a relative sense. Thus the various derivatives of C-terminally his tagged Zea mays bx8 SEQ ID No:2 were cloned and expressed in E. coli BL21 (DE3) as described in Example 1 and assayed in plates as crude extracts, as in Example 2, but monitored by LCMS rather than the luminescence assay. Active derivatives that exhibited detectable activity with herbicide V were the I374V, H376C; V367I, I374V; A246T, E256V; V367I, H376C and the E256V, R265Q double mutants and, the D170E, A72P, A174P triple mutant. In addition the hybrid sequences SEQ ID NO: 50, SEQ ID NO: 51 and SEQ ID NO: 58 were also active with herbicide V. Of these the most active (estimated to be about a third to a half the activity of the like-expressed Zea mays bx9 containing E. coli extract) bx8 derivatives with respect to herbicide V were the 1374, H376 (SEQ ID NO: 40) double mutant of bx8 and SEQ ID NO: 50. However, especially since the bx9-based ELISA did not work reliably with the bx8 derivatives the activities could not be compared on a quantitative basis. SDS PAGE suggested that the bx8 derivatives were generally expressed less strongly than the Zea mays bx9 w/t control protein in the crude extracts and so it is likely that the true specific activity of some of these bx8 derivatives was at a level similar to or more than the activity of bx9.

### EXAMPLE 5

### Identification of zmBX8/zmBX9 Orthologues from Various Species

SEQ ID Nos: 1 and 2 were used to search plant sequence databases for orthologues of the zmBX8 and zmBX9 sequences using either BlastP (X) or TBlastX (X). Sequences were recovered from a number of species which were mainly grasses although some dicot orthologues were recovered. Some of these polypeptide sequences are depicted and aligned in FIG. 1 and they are also listed as SEQ ID 1-15 herein. The sequence identity to zmBX9 ranged from e.g. about 73% (Zea mays BX8 without any adjustment to minimize gap penalties) to about 30% (Larkspur bx-like polypeptide) respectively (based on AlignX in Vector NTI at default parameter settings).

### EXAMPLE 6

### Cloning, Expression and Assay of Various BX8 and BX9 Orthologues

DNA sequences, optimized for E. coli codon usage, corresponding to SEQ IDs 1-15, encoding BX8 and BX9 orthologue polypeptides derived from a range of species (as depicted in FIG. 1) were synthesized by Genewiz (South Plainfield, USA) with 5′ NdeI and 3′ XhoI restriction sites. The various orthologues were synthesized with either a N-terminal 6×His purification tag or C-terminal 6×His purification tag (i.e. tried both ways to achieve best expression of activity) and cloned into the E. coli expression plasmid pET24a (Novagen) via the NdeI and XhoI restriction sites. Expression, purification and assay was as described in Example 1.

Assays were carried out as described in Example 1. Partly purified C-terminally his tagged w/t Larkspur enzyme (SEQ ID No 10 with a 6 amino acid C-terminal tag) catalyzed glucosyl transfer to herbicide V at a rate estimated to be about 0.0065/s/pmol at 1 mM herbicide V. The similarly-tagged w/t bx-like enzymes from rye (SEQ ID No 11) and from wheat (SEQ ID No 4) were also active but only at levels close to the detection limit at ˜0.0015/s/pmol. Further examples of assay results from further experiments are depicted in Table 14. All of the proteins were C-terminally his tagged versions of the sequences referenced except for the Zea mays bx8 SEQ ID NO: 2 which was N-terminally his tagged. In these experiments the limit of detection of activity was about 0.001/s/pmol of enzyme and low numbers below 0.003 can only be taken as indicative of relative rankings rather than absolutely accurate and especially in assays of Zea mays bx8 which exhibited a relatively high DMSO reagent background rate.

TABLE 14
Activities with various alcohol and aminal herbicides tested as substrates of w/t
bx glucosyl transferases from various species.

| | | SEQ ID | SEQ ID | SEQ ID | SEQ ID | SEQ ID | SEQ ID |
|---|---|---|---|---|---|---|---|
| | | NO 1 | NO 2 | NO 4 | NO 8 | NO 10 | NO 10 |
| | Herbicide | Activity | Activity | Activity | Activity | Activity | Activity |
| | concen- | (pmol/sec/ | (pmol/sec/ | (pmol/sec/ | (pmol/sec/ | (pmol/sec/ | (pmol/sec/ |
| | tration | pmol | pmol | pmol | pmol | pmol | pmol |
| Compound | (mM) | enzyme) | enzyme) | enzyme) | enzyme) | enzyme) | enzyme) |
| V | 0.500 | 0.005 | 0.000 | 0.001 | 0.002 | 0.006 | 0.009 |
| VI | 0.500 | 0.013 | 0.000 | 0.000 | 0.002 | 0.007 | 0.011 |
| XVIII | 0.500 | 0.002 | 0.001 | 0.000 | 0.003 | 0.006 | 0.009 |
| XI | 0.500 | 0.000 | 0.001 | 0.001 | 0.002 | 0.003 | 0.007 |
| XXI | 0.500 | | | | | | 0.013 |

### EXAMPLE 7

### Cloning, Expression, Purification and Assay of Various Mutants and Combinations of Mutants of bx Type Glucosyl Transferase Polypeptides from Various Species

DNA sequences, optimized for E. coli codon usage, were cloned, expressed and the various proteins purified and assayed as described in the foregoing examples. The results of these assays are set out in Tables 15. Using the UDP assay (as described in examples 1, 3 and 6) the data obtained from the Zea mays bx8 w/t protein and its variants was noisy due to relatively high background rates in DMSO reagent blanks. The values reported in the table 15 were thus monitored by LC/MS as described in example 1.

TABLE 15
Relative activities of various w/t and mutant bx-type
glucosyl transferases with various alcohol and aminal herbicides.
Assays were run as described in Example 1. The numbers in the
table represent the integrated peak areas of the beta-glucoside
products of the enzyme catalyzed reaction with the various
herbicides where ‘nd’ means ‘not detectable’ and a
space means that no experiment was carried out. The LC/MS
peak areas of the herbicide glucoside conjugates only provide
accurate relative quantifications of the amount formed in the assay
after about 60 min and with ~15 pmol of enzyme.

| | Test structure | | |
|---|---|---|---|
| SEQ ID | XI | VI | V |
| 2 | 1.90E+04 | 8.08E+06 | 2.52E+06 |
| 2 | 1.05E+04 | 7.57E+06 | 2.97E+06 |
| 2 | 2.36E+04 | 8.81E+06 | |
| 2 | 1.84E+04 | 7.78E+06 | |
| 31 | nd | 8.84E+06 | 5.57E+06 |
| 31 | nd | 1.04E+07 | 5.59E+06 |
| 32 | 9.98E+05 | 1.75E+08 | 8.33E+06 |
| 32 | 1.08E+06 | 1.89E+08 | 6.18E+06 |
| 33 | 1.70E+05 | 3.59E+07 | 9.27E+06 |
| 33 | 3.50E+05 | 3.45E+07 | 9.76E+06 |
| 34 | 2.45E+05 | 2.17E+07 | 5.21E+06 |
| 34 | 2.45E+05 | 2.00E+07 | 4.16E+06 |
| 35 | 3.66E+05 | 8.20E+07 | 5.96E+06 |
| 35 | 1.71E+05 | 2.22E+07 | 4.40E+06 |
| 36 | 6.03E+06 | 6.40E+09 | 1.75E+08 |
| 36 | 6.40E+06 | 6.87E+09 | 1.96E+08 |
| 4 | nd | nd | nd |
| 4 | nd | nd | nd |
| 4 | nd | nd | nd |
| 4 | nd | nd | nd |
| 43 | nd | nd | 3.88E+05 |
| 43 | nd | nd | 2.26E+05 |
| 45 | nd | nd | 1.56E+05 |
| 45 | nd | nd | 9.51E+04 |
| 46 | nd | nd | 2.35E+04 |
| 46 | nd | nd | 9.29E+03 |
| 47 | nd | 1.68E+05 | 8.67E+05 |
| 47 | nd | 1.05E+05 | 6.30E+05 |

It is clear from the increased activities of various of the mutants over the wild type proteins in respect of various of the herbicide chemistries that the same equivalent mutations that were found useful to improve the activity of Zea mays bx9 are also useful to increase the activity of the Zea mays bx8 (SEQ ID No: 2) and wheat (SEQ ID No: 4) bx glucosyl transferases. The improvements were particularly striking in cases where the activity of the corresponding w/t protein was not even detectable.

### EXAMPLE 8

### Generation of Variant Sequences of the Zea mays BX9 Glucosyltransferase Gene and Assay with Metribuzin as Acceptor Substrate

Assays were carried out on extracts of E. coli BL21 DE3 expressing the various C-terminally his-tagged library variants of SEQ ID NO:1 at the amino acid positions listed in Table 11 as described in Example 2 except, in this case, with 2 mM metribuzin in place of herbicide V.

Activities observed with metribuzin were generally lower in magnitude than seen with herbicide V in the previous examples. For example, in one plate assay run with 2 mM metribuzin using 5 ul of diluted extract of plate-grown BL21DE3 cells expressing w/t bx9 and assayed for 40 minutes (as described in Example 2) the UDP-Glo luminescence signal was ˜3.4 E6 as compared with ˜2.3E6 in the DMSO control and ˜1.7E6 in the H24A null mutant control. Thus, after subtracting the H24A null background, the metribuzin ‘signal’ was only 2-2.5× greater than the DMSO control background signal. It was therefore important to assess potential improvements in the metribuzin activity of the various mutants relative to the wild type bx9 enzyme based not only on the H24A null activity control but also based upon controls with DMSO in place of metribuzin acceptor substrate. In some plate assays (using more dilute extracts) the metribuzin signals from the w/t enzyme was the same or barely greater than the DMSO control. Additionally, some mutations gave an increase in the ‘background’ UDP-glucose hydrolysis activity with just DMSO present that was the same or similar to the magnitude of the increase in signal seen with metribuzin. In these instances it is therefore uncertain from just this UDP-Glo assay method whether the specific metribuzin signal (glucosylation of metribuzin) is improved or not i.e. the signal seen with metribuzin may or may not all or to a large part be attributable to increased background hydrolysis). On the other hand, in those cases where the metribuzin signal is either significantly greater or indeed significantly smaller than that seen with just DMSO it is then clearer that a significant proportion of the signal is likely to be genuinely due to metribuzin glucosylation. Thus the ratio of the metribuzin to DMSO signal as well as the increase in signal relative to the w/t bx9 is used to help distinguish those mutations most likely to offer the highest activity to metribuzin. A follow up assay monitored by, for example, LC MS is one method to distinguish these cases more quantitatively. The tables below summarise the results from two different sets of luminescence plate assays to detect single mutations giving improvements in the magnitude of the signal with metribuzin relative to w/t bx9 and/or the apparent specificity for metribuzin as measured by the deviation of the ratio of metribuzin to DMSO control activity from the bx9 w/t value of around unity.

TABLE 16a
Luminescence assay results for mutants at positions 19, 117, 135,
279 and 334 of SEQ ID No: 1 assayed with 2 mM metribuzin
The first and second columns are the luminescence signals observed
with 2 mM metribuzin and in the DMSO control, respectively. The
figures in the 3^{rd} column are the metribuzin signals for each mutant
divided by the metribuzin signal of w/t bx9. The figures in the 4^{th}
column are the ratios of the metribuzin signal (column 1) to the
DMSO signal (column 2) of each mutant. H24A is the null
mutant background control.

| | metribuzin | DMSO | SIGNAL | RATIO |
|---|---|---|---|---|
| bx9 w/t | 1.23E+05 | 1.27E+05 | 1.00 | 0.97 |
| F19M | 1.07E+05 | 8.30E+04 | 0.86 | 1.28 |
| S117G | 1.56E+05 | 1.35E+05 | 1.26 | 1.15 |
| M135V | 1.46E+05 | 1.50E+05 | 1.18 | 0.97 |
| M279W | 1.41E+05 | 9.89E+04 | 1.14 | 1.42 |
| A334S | 1.21E+05 | 1.27E+05 | 0.98 | 0.95 |
| A334E | 1.54E+05 | 1.60E+05 | 1.25 | 0.96 |
| A334T | 1.54E+05 | 1.62E+05 | 1.25 | 0.95 |
| A334N | 1.93E+05 | 1.38E+05 | 1.56 | 1.39 |
| A334R | 2.39E+05 | 2.66E+05 | 1.94 | 0.90 |
| A334C | 1.39E+05 | 1.48E+05 | 1.13 | 0.94 |
| A432P, M279F | 2.08E+05 | 1.26E+05 | 1.68 | 1.65 |
| H24A | 1.00E+05 | 1.00E+05 | | |

TABLE 16b
Luminescence assay results for mutants at various positions of
SEQ ID No: 1 assayed with 2 mM metribuzin
The first and second columns are the luminescence signals observed
with 2 mM metribuzin and in the DMSO control, respectively. The
figures in the 3^{rd} column are the metribuzin signals for each
mutant divided by the signal of w/t bx9. The figures in the 4^{th}
column are the ratios of the metribuzin signal (column 1) to
the DMSO signal (column 2) of each mutant. H24A is the
null mutant background control.

| | metribuzin | DMSO | SIGNAL | RATIO |
|---|---|---|---|---|
| bx9 w/t | 2.80E+05 | 2.76E+05 | 1.00 | 1.01 |
| F19M | 2.76E+05 | 1.92E+05 | 0.99 | 1.44 |
| I78Y | 3.44E+05 | 3.27E+05 | 1.23 | 1.05 |
| I81Y | 2.53E+05 | 2.25E+05 | 0.90 | 1.12 |
| I81L | 1.87E+05 | 1.68E+05 | 0.67 | 1.11 |
| S117I | 5.56E+05 | 5.14E+05 | 1.99 | 1.08 |
| S117V | 3.30E+05 | 3.38E+05 | 1.18 | 0.98 |
| S117G | 3.00E+05 | 2.41E+05 | 1.07 | 1.24 |
| M136F | 2.59E+05 | 2.57E+05 | 0.93 | 1.00 |
| L143W | 2.72E+05 | 2.42E+05 | 0.97 | 1.12 |
| L143Y | 2.98E+05 | 2.77E+05 | 1.06 | 1.07 |
| L143F | 3.45E+05 | 3.18E+05 | 1.23 | 1.09 |
| L143M | 2.86E+05 | 2.74E+05 | 1.02 | 1.04 |
| V199M | 2.58E+05 | 2.53E+05 | 0.92 | 1.02 |
| A202S | 2.53E+05 | 2.70E+05 | 0.91 | 0.94 |
| A202T | 2.61E+05 | 2.52E+05 | 0.93 | 1.04 |
| T220F | 6.21E+05 | 7.87E+05 | 2.22 | 0.79 |
| T220H | 5.08E+05 | 5.47E+05 | 1.82 | 0.93 |
| T220W | 4.44E+05 | 4.99E+05 | 1.59 | 0.89 |
| T220P | 4.40E+05 | 5.28E+05 | 1.57 | 0.83 |
| M279W | 3.51E+05 | 2.09E+05 | 1.26 | 1.68 |
| A281K | 1.39E+06 | 1.66E+06 | 4.98 | 0.84 |
| A281M | 7.25E+05 | 8.45E+05 | 2.59 | 0.86 |
| A281Q | 7.89E+05 | 7.59E+05 | 2.82 | 1.04 |
| A281C | 4.43E+05 | 4.80E+05 | 1.58 | 0.92 |
| A281R | 1.09E+06 | 1.30E+06 | 3.90 | 0.84 |
| A334P | 3.48E+05 | 3.97E+05 | 1.24 | 0.88 |
| A334V | 4.40E+05 | 4.80E+05 | 1.57 | 0.92 |
| A334Q | 3.40E+05 | 4.88E+05 | 1.22 | 0.70 |
| A334L | 4.22E+05 | 5.19E+05 | 1.51 | 0.81 |
| A334I | 2.94E+05 | 3.29E+05 | 1.05 | 0.89 |
| I363L | 3.47E+05 | 4.00E+05 | 1.24 | 0.87 |
| I363M | 4.68E+05 | 5.22E+05 | 1.67 | 0.90 |
| C372L | 3.41E+05 | 3.22E+05 | 1.22 | 1.06 |
| G376L | 3.56E+05 | 3.59E+05 | 1.27 | 0.99 |
| G376M | 3.15E+05 | 2.80E+05 | 1.13 | 1.13 |
| A432P, M279F | 7.06E+05 | 2.75E+05 | 2.52 | 2.56 |
| H24A | 2.39E+05 | 2.60E+05 | | |

Based on the data in the two tables, listed below are some mutants of particular interest. These yielded an increased signal relative to the 2 mM bx9 w/t control signal (by a factor corresponding to the figure bounded by the first set of parentheses following the mutant identification) and/or exhibited a ratio of the activity with metribuzin relative to the activity with DMSO deviating significantly from 1.0 (ratios given by the figure within the second set of parentheses). Examples of preferred mutants at the various positions therefore included F19M (0.93) (1.36), S117G (1.17)(1.20), T220P (1.57) (0.83) M279W (1.20)(1.55), A334N (1.56) (1.39) and A281K (4.98) (0.84). It will be noted that the double mutant, A432P; M279F (2.1) (2.1), provided the highest combined activity and specificity in respect of metribuzin. Note also that because the constant reagent background (as indicated by the H24A zero activity mutant background) constituted a large and fixed part of the total signal, the above-described way of deriving the first and second parameters represents a conservative estimate of the improvement in the metribuzin activity of any given mutant over the w/t. For example, when this fixed H24A background signal is subtracted, it can be estimated that S117G (1.17) (1.20) in fact corresponds to a roughly 2× improvement in total activity associated with a 1.6× improvement in the specific activity to metribuzin relative to the w/t bx9 enzyme.

In a separate screen carried out as above, a further library of mutations at position 432 was screened and multiple substitutions for the alanine at this position in the wild type found to exhibit increased activity and to also exhibit an increased ratio of activity with 2 mM metribuzin over the DMSO background rate. Thus for example, A432P (3.5)(1.35); A432R (5)(1.35); A432H (5) (1.35); A432Q (6) (1.35); A432T (3.5) (1.35) and A432L (6) (1.30) were significantly improved. Some mutations at this position result in significantly reduced expression of the glucosyl transferase enzyme in E. coli (as monitored by ELISA) meaning that the specific activity improvements over bx9 expressed on a per ng of protein basis were even greater. For example A432D was expressed at about 65% of the level of bx9 and A432T at only ˜40%.

Mutant combinations near optimal for one herbicide can be highly selective and relatively ineffective for other herbicides. For example SEQ ID No:17 which combines the M279F, S117V and A334K mutations is some ˜2000× improved over bx9 w/t enzyme in respect of the kcat/Km value in respect of herbicide VI but exhibits only a slight (˜1.3-1.5×) increase over the low level of activity of the bx9 w/t protein in respect of metribuzin.

A further separate screen was carried out to explore a library of additional mutations at various positions within the context of SEQ ID No: 17 for improved metribuzin activity. These were assayed, assessed and scored as above. This yielded the following 4 mutants of particular interest where there was found both a significant improvement in the magnitude of the metribuzin signal over the SEQ ID No:17 control and also where the ratio of the metribuzin to DMSO signal was significantly different from 1.0. These were S75K (2.5)(1.25), A236G (4.0)(1.15), A433V (3.3)(9.0) and R449C (1.8)(1.2).

A yet further and more comprehensive screen was carried out to explore saturation mutagenesis at all remaining amino acid positions within the context of SEQ ID No:17 for improved metribuzin activity. Again these were assayed, assessed and scored as above and the results of (out of the thousands screened) of the subset that were of interest are summarized in Table 16.

TABLE 17
Luminescence assay results for mutants at various positions of
SEQ ID No: 17 assayed with 2 mM metribuzin
Control refers to SEQ ID No: 17. The first and second columns are
the luminescence signals observed with 2 mM metribuzin and in the
DMSO control, respectively. The figures in the 3^{rd} column are
the metribuzin signals for each mutant divided by the signal of
the SEQ ID No. 17 control. The figures in the 4^{th} column are
the ratios of the metribuzin signal (column 1) to the DMSO
signal (column 2) of each mutant.

| mutant | METRIBUZIN | DMSO | SIGNAL | ratio |
|---|---|---|---|---|
| control | 1.82E+06 | 1.57E+06 | 1.00 | 1.16 |
| F213W | 8.66E+06 | 6.96E+06 | 4.75 | 1.24 |
| F213Y | 4.48E+06 | 3.29E+06 | 2.46 | 1.36 |
| P214D | 6.67E+06 | 4.99E+06 | 3.66 | 1.34 |
| L215H | 4.44E+06 | 3.25E+06 | 2.44 | 1.37 |
| L215S | 5.65E+06 | 4.28E+06 | 3.09 | 1.32 |
| L215D | 5.72E+06 | 4.26E+06 | 3.14 | 1.34 |
| I216Y | 6.38E+06 | 4.79E+06 | 3.50 | 1.33 |
| I216W | 4.99E+06 | 3.70E+06 | 2.73 | 1.35 |
| A234T | 5.30E+06 | 4.53E+06 | 2.91 | 1.17 |
| A234K | 4.23E+06 | 4.58E+06 | 2.32 | 0.92 |
| A234L | 5.49E+06 | 4.48E+06 | 3.01 | 1.22 |
| A234C | 5.40E+06 | 4.20E+06 | 2.96 | 1.29 |
| A234P | 3.71E+06 | 2.89E+06 | 2.03 | 1.28 |
| P284Y | 3.30E+06 | 2.85E+06 | 1.81 | 1.16 |
| P284F | 3.30E+06 | 2.55E+06 | 1.81 | 1.29 |
| P284W | 2.97E+06 | 2.31E+06 | 1.63 | 1.29 |
| W303L | 4.20E+06 | 3.10E+06 | 2.30 | 1.35 |
| F313K | 2.41E+06 | 1.85E+06 | 1.32 | 1.30 |
| F313M | 3.48E+06 | 2.69E+06 | 1.91 | 1.29 |
| F313G | 2.47E+06 | 2.19E+06 | 1.35 | 1.12 |
| E339V | 4.81E+06 | 3.18E+06 | 2.64 | 1.51 |
| E339A | 3.66E+06 | 2.39E+06 | 2.01 | 1.53 |
| L351F | 2.40E+06 | 2.01E+06 | 1.32 | 1.19 |
| V360M | 3.68E+06 | 2.88E+06 | 2.02 | 1.28 |
| S364I | 5.16E+06 | 4.32E+06 | 2.83 | 1.20 |
| S364N | 3.74E+06 | 2.82E+06 | 2.05 | 1.33 |
| S364L | 6.00E+06 | 5.04E+06 | 3.29 | 1.19 |
| G366C | 5.19E+06 | 3.83E+06 | 2.84 | 1.35 |
| G366R | 5.82E+06 | 4.35E+06 | 3.19 | 1.34 |
| G366H | 6.17E+06 | 4.62E+06 | 3.38 | 1.34 |
| H375F | 8.59E+06 | 5.83E+06 | 4.71 | 1.47 |
| H375L | 5.50E+05 | 5.95E+06 | 0.30 | 0.09 |
| H375Y | 6.93E+06 | 2.97E+06 | 3.80 | 2.33 |
| G418M | 3.97E+06 | 3.10E+06 | 2.18 | 1.28 |
| G418R | 5.36E+06 | 3.86E+06 | 2.94 | 1.39 |
| G418Y | 5.67E+06 | 3.86E+06 | 3.11 | 1.47 |
| E423I | 2.67E+06 | 2.28E+06 | 1.46 | 1.17 |
| R424P | 4.58E+06 | 4.02E+06 | 2.51 | 1.14 |
| R424S | 1.46E+06 | 8.84E+05 | 0.80 | 1.65 |
| M425Y | 6.07E+06 | 4.37E+06 | 3.33 | 1.39 |
| M425F | 7.53E+06 | 3.84E+06 | 4.13 | 1.96 |
| K426E | 2.49E+06 | 1.98E+06 | 1.36 | 1.26 |
| K429S | 4.34E+06 | 3.34E+06 | 2.38 | 1.30 |
| K429N | 3.33E+06 | 2.47E+06 | 1.83 | 1.35 |
| I430P | 5.15E+06 | 3.83E+06 | 2.82 | 1.34 |
| A431P | 5.12E+06 | 3.87E+06 | 2.81 | 1.32 |
| A431G | 3.67E+06 | 2.69E+06 | 2.01 | 1.37 |
| A432Y | 8.07E+06 | 6.45E+06 | 4.42 | 1.25 |
| A432T | 6.51E+06 | 5.56E+06 | 3.57 | 1.17 |
| A432V | 7.07E+06 | 5.69E+06 | 3.88 | 1.24 |
| A432M | 6.25E+06 | 5.11E+06 | 3.43 | 1.22 |
| A432H | 6.28E+06 | 6.08E+06 | 3.44 | 1.03 |
| A432N | 7.67E+06 | 6.13E+06 | 4.21 | 1.25 |
| A432Q | 7.46E+06 | 6.22E+06 | 4.09 | 1.20 |

Mutants of particular interest (showing the highest metribuzin signals relative to the control combined with the highest ratios of metribuzin to DMSO activity) include F213W(4.7)(1.24); P214D(3.7)(1.34); L215D(3.1)(1.34); I216Y(3.5)(1.33); A234C(3.0)(1.29); P284F(1.8)(1.29); W303L(2.3)(1.35); F313M(1.9)(1.30); E339V(2.6)(1.51); V360M(2.0)(1.28); S364L(3.3)(1.20); G366H(3.4)(1.34); H375Y(3.8)(2.33); G418Y(3.1)(1.47); R424P(2.5)(1.14); M425F(4.1)(1.96); K429S(2.4)(1.30); I430P(2.8)(1.34); A431P(2.8)(1.32) and A432Y(4.4)(1.25).

Positions identified as of particular interest with respect to SEQ ID NO: 1 (wild type bx9) with respect to mutation towards metribuzin acceptor substrate activity can therefore be summarized and listed as follows:
F19, F21, S75, S117, L194, F213, P214, L215, I216, T220, A234, A236, A281, P284, M279, W303, F313, A334, E339, L351, V360, I363, S364, G366, H375, G418, E423, M425, K426, K429, 1430, A432, A433 and R449

Combining mutations together often has the effect of not only increasing activity versus a given acceptor substrate but also of increasing specificity for that substrate and increasing discrimination with respect to both the DMSO background activity and activity versus other substrates. For example some combinations with mutations at position 432 were also found to be effective. Thus, for example, tested in the same crude extract and luminescence plate assay method, the double mutant, A432P, M279F of bx9 exhibited a similar DMSO reagent background rate to the w/t bx9 sequence but an ˜2.0× fold increase activity with 2 mM metribuzin (i.e. A432P, M279F (2.1) (2.1) and, assayed as above, exhibited a superior discrimination ratio (2.1) over bx9 than either single mutant. Furthermore ELISA assay (as described in example 2) indicated that this C-terminally his-tagged A432P, M279F variant of bx9 was expressed at only—10-20% of the level of the w/t protein. Thus, expressed per ng of bx protein, the A432P, M279F double mutant exhibits 20 or so fold-greater activity versus metribuzin than the w/t enzyme.

Assayed and scored as above, other examples of mutant combinations exhibiting clearly improved metribuzin activity and specificity over the w/t enzyme are : F21Y, T220P, M279F, A281K, L194V (>4.0) (>2.2); F21Y, T220W, M279F, A281K, L194V (>4.0) (>2.2), F21Y, T220P, M279F, A281K, L194C (>4.0) (>2.2); F21Y, T220W, M279F, A281K, L194C (>4.0) (>2.2), T220P, M279F, A281K, L194V (>4.0) (>2.2); T220W, M279F, A281K, L194V (>4.0) (>2.2); T220P, M279F, A281K, L194C (>4.0) (>2.2) and T220W, M279F, A281K, L194C (>4.0) (>2.2). The ‘>’ symbol in the above lists reflects the fact that the signal observed in these particular assays with 2 mM metribuzin was above the threshold for near-linear detection using the UDP-Glo assay.

Further examples of combinations which, when assayed and scored as above, exhibited improved metribuzin activity and specificity over the w/t enzyme are: S117G, M279W, E339A(5.0)(6.5); S117G, M279W, E339V (7.5)(5.0); M279F, E339A, H375Y (7.5)(7.0); M279F, H375Y (5.0) (6.0); M279W, H375Y (4.0)(5.0) and M279F, H375F (5.0)(4.0).

The C terminally his tagged, M279F, E339A, H375Y triple mutant of SEQ ID NO: 1 was cloned, expressed and purified as described for SEQ ID NO: 1 in Example 1. The purified protein was assayed using the UDP luminescence assay as described in Example 1 but with 0.5 mM UDP-glucose and varying concentrations of metribuzin as acceptor substrate. Best fit values of kcat, Km and kcat/Km are obtained by direct fitting of the data to the Michaelis-Menten equation using Graphpad Prism™ software. Assays were run for 10 minutes with ˜2.5 pmol of enzyme. The low uncoupled rate of UDP-glucose in the absence of acceptor substrate (0.03 pmol/s) observed was ignored based on the reasonable assumption that addition of herbicide substrate should completely displace and inhibit this uncoupled reaction. The kcat value was estimated as 0.19/s (95% confidence limits to 0.178-0.208/s) and Km value for metribuzin as 0.54 mM (95% confidence limits 0.45-0.62 mM) and kcat/Km therefore—0.35/mM/s.

The C terminally his tagged, S117G, M279W, E339V triple mutant of SEQ ID NO: 1 was cloned, expressed and purified as described for SEQ ID NO: 1 in Example 1. The purified protein was assayed using the UDP luminescence assay as described in Example 1 but with 0.5 mM UDP-glucose and either 2 mM metribuzin or a saturated solution of the R-enantiomer of triaziflam herbicide as acceptor substrate. Assayed similarly to as above, kcat/Km was estimated as ˜0.20/mM/s in respect of metribuzin (estimated over a range of concentrations from 0.125 to 2 mM metribuzin) and greater than ˜0.05/mM/s in respect of R triaziflam.

### EXAMPLE 9

### Modifications of bx Proteins to Improve Herbicide Substrate Acceptor Activity by Including a Further Peptide Loop

DNA sequences, optimized for E. coli codon usage, are cloned, expressed and the various proteins purified and assayed as described in the foregoing examples. As in Example 4, a DNA sequence is designed and synthesized to express the N-terminally his tagged Zea mays bx8 (SEQ ID No: 2). However in this case the DNA sequence is modified to further include a peptide insertion “GIGVD”=SEQ ID No: 102 in place of D442 of SEQ ID No: 2 and as indicated in Table 2. The resultant modified sequence is cloned into the E. coli expression plasmid pET24a using 5′ NdeI and 3′ XhoI restriction sites, expressed, purified and assayed. It is found that this mutant protein containing the peptide insert (SEQ ID NO: 102) exhibits a somewhat increased glucosyl transferase activity in in vitro assays with herbicide V as acceptor substrate as compared with the unmodified w/t Zea mays bx8. In LC/MS assays run for 30 min and similar to those described in example 7 the integrated peak areas for glucoside product from herbicide V from the w/t Zea mays bx8 SEQ ID NO:2 was 1.5E6 units whereas the corresponding number for the equivalent protein containing the GIGVD peptide insert was 2.5E6 units. SEQ ID NO: 37 is an example of a polypeptide sequence where a polypeptide insertion, D442(GIGVDVD), (SEQ ID NO 104) has been inserted into a triple mutant S121V, M283F, S338K Zea mays bx8 sequence. Similarly, SEQ ID NO:48 is an example of a polypeptide sequence where a polypeptide insertion, N437(GIGVDVD, (SEQ ID NO 104) has been inserted into a double mutant L278F, S333K wheat bx sequence.

### EXAMPLE 10

### Herbicide Tolerance Conferred by Heterologous BX Glucosyltransferase Enzymes Expressed in Tobacco

In the present example, Zea mays BX8 or BX9 or orthologues of BX8/9, for example SEQ ID Nos. 1-59 and various herbicide-active mutations and combinations of mutations thereof (e.g. as listed in Tables 1-9) are expressed in transgenic tobacco. DNA sequences that encode these polypeptides (optimized for tobacco or, optionally, codon optimized according to a target crop such as soybean) are prepared synthetically and obtained commercially from Genewiz (South Plainfield, USA). Each sequence is designed to include a 5′ fusion with TMV omega 5′ leader (SEQ ID NO: 109). The DNA sequences are flanked at the 5′ end with XhoI and at the 3′ end with KpnI to facilitate direct cloning into a suitable binary vector for Agrobacterium-based plant transformation.

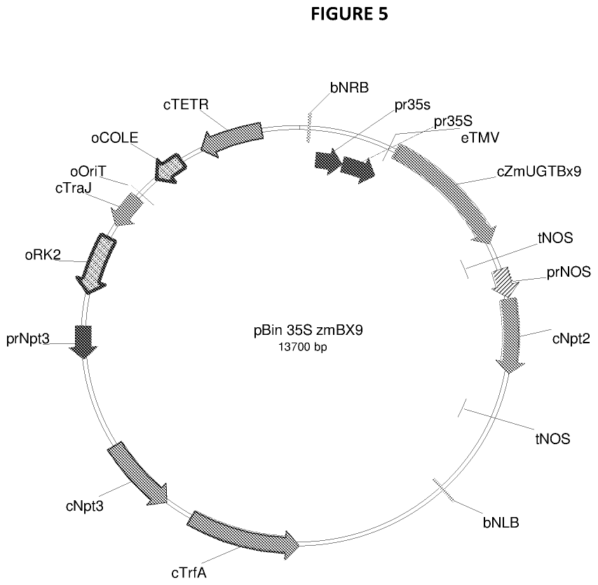

In a particular example, the expression cassette, comprising the TMV omega 5′ leader and a BX encoding gene of interest is excised using XhoI/KpnI and cloned into similarly digested pBIN 19 (Bevan, Nucl. Acids Res. (1984) behind a double enhanced 35S promoter (SEQ ID NO:110) and ahead of a NOS 3′ transcription terminator (SEQ ID NO:111) and then transformed into E. coli DH5 alpha competent cells (see FIG. 5). DNA recovered from the E. coli is used to transform Agrobacterium tumefaciens LBA4404, and the transformed bacteria are selected on media contain rifampicin and kanamycin. Tobacco tissue is subjected to Agrobacterium-mediated transformation using methods well described in the art or as described herein. For example, a master plate of Agrobacterium tumefaciens containing the BX glucosyltransferase expressing binary vector is used to inoculate 10 ml LB (L broth) containing 100 mg/l Rifampicin plus 50 mg/l Kanamycin using a single bacterial colony. This is incubated overnight at 28° C. shaking at 200 rpm. This entire overnight culture is used to inoculate a 50 ml volume of LB containing the same antibiotics. Again this is cultured overnight at 28° C. shaking at 200 rpm. The Agrobacterium cells are pelleted by centrifuging at 3000 rpm for 15 minutes and then resuspended in MS (Murashige and Skoog) medium containing 30 g/l sucrose, pH 5.9 to an OD (600 nM)=0.6. This suspension is dispensed in 25 ml aliquots into petri dishes.

Clonally micro-propagated tobacco shoot cultures are used to excise young (not yet fully expanded) leaves. The mid rib and outer leaf margins are removed and discarded, and the remaining lamina cut into 1 cm squares. These are transferred to the Agrobacterium suspension for 20 minutes. Explants are then removed, dabbed on sterile filter paper to remove excess suspension, then transferred onto solid NBM medium (MS medium containing 30 g/l sucrose, 1 mg/l BAP (benzylaminopurine) and 0.1 mg/l NAA (napthalene acetic acid) at pH 5.9 and solidified with 8 g/l Plantagar), with the abaxial surface of each explant in contact with the medium. Approximately 7 explants are transferred per plate, which are then sealed and maintained in a lit incubator at 25° C. for a 16 hour photoperiod for 3 days.

Explants are then transferred onto NBM medium containing 100 mg/l Kanamycin plus antibiotics to prevent further growth of Agrobacterium (200 mg/l timentin with 250 mg/l carbenicillin). Further subculture onto this same medium was then performed every 2 weeks.

As shoots start to regenerate from the callusing leaf explants, these are removed to Shoot elongation medium (MS medium, 30 g/l sucrose, 8 g/l Plantagar, 100 mg/l Kanamycin, 200 mg/l timentin, 250 mg/l carbenicillin, pH 5.9). Stable transgenic plants readily root within 2 weeks. To provide multiple plants per event to ultimately allow more than one herbicide test per transgenic plant, all rooting shoots are micropropagated to generate 3 or more rooted clones.

Putative transgenic plants that are rooting and showing vigorous shoot growth on the medium incorporating Kanamycin are analysed by PCR using primers that amplified a 500 bp fragment specific to the BX glucosyltransferase transgene of interest. Evaluation of this same primer set on untransformed tobacco showed conclusively that these primers would not amplify any sequences from the native tobacco genome.

Transformed shoots are divided into 2 or 3 clones and regenerated from kanamycin resistant callus. Shoots are rooted on MS agar containing kanamycin. Surviving rooted explants are re-rooted to provide approximately 40-50 kanamycin resistant and PCR positive events from each event.

Once rooted, plantlets are transferred from agar and potted into 50% peat, 50% John Innes Soil No. 3 with slow-release fertilizer in 3 inch round pots and left regularly watered to establish for 8-12d in the glass house. Glass house conditions are about 24-27° C. day; 18-21° C. night and approximately a 14h photoperiod. Humidity is adjusted to ˜65% and light levels used are up to 2000 μmol/m² at bench level.

Three transgenic populations of about forty tobacco plants and comprising, a glucosyl transferase gene encoding either zmBX8 (SEQ ID NO 2) or zmBX9 (SEQ ID NO 1) were thus produced. A sub-set of about 30 plants were selected on the basis of similar size from each population for spray testing. The plants were then sprayed with 30 g/ha of Compound VI. VI was mixed in water with 0.2-0.25% X-77 surfactant and sprayed from a boom on a suitable track sprayer moving at 2 mph with the nozzle about 2 inches from the plant tops. Spray volume was 200 l/ha. Plants were assessed for damage and scored at 7 and 14 days after treatment (DAT). The results are depicted in Table 18. It is clear that in comparison to the wild type tobacco controls, several transgenic lines such as 6266, 6164 and 2302 from the tobacco population overexpressing the zmBX9 gene SEQ ID No 1 demonstrate tolerance to herbicide VI. In good accord with the in vitro data (table 14) the tobacco population likewise expressing the zmBX8 gene exhibited little or no tolerance to herbicide VI.

TABLE 18
GH evaluation of percent damage to w/t/and transgenic
tobacco plants expressing either SEQ ID No 1 or
SEQ ID No 2 at 14 DAT with 30 g/ha of compound VI

| SEQ ID | | SEQ ID | | | wild |
|---|---|---|---|---|---|
| No 2 | SEQ ID | No 1 | SEQ ID | Wild | type |
| line | No 2 | line | No 1 | Type | plant |
| number | results | number | results | plants | results |
| 6159 | 100 | 6249 | 85 | A | 75 |
| 6161 | 100 | 6251 | 90 | B | 95 |
| 6163 | 70 | 6252 | 20 | C | 85 |
| 6164 | 100 | 6256 | 10 | | |
| 6167 | 100 | 6258 | 100 | | |
| 6169 | 100 | 6259 | 100 | | |
| 6170 | 90 | 6262 | 15 | | |
| 6171 | 100 | 6266 | 10 | | |
| 6172 | 100 | 6269 | 65 | | |
| 6173 | 100 | 6273 | 100 | | |
| 6174 | 100 | 6274 | 90 | | |
| 6175 | 100 | 6276 | 100 | | |
| 6176 | 75 | 6278 | 100 | | |
| 6178 | 100 | 6280 | 40 | | |
| 6179 | 100 | 6281 | 15 | | |
| 6181 | 95 | 6282 | 95 | | |
| 6189 | 100 | 6283 | 75 | | |
| 6190 | 100 | 6284 | 100 | | |
| 6191 | 100 | 6285 | 100 | | |
| 6193 | 100 | 6286 | 60 | | |
| 6194 | 100 | 6287 | 25 | | |
| 6196 | 70 | 6288 | 65 | | |
| 6197 | 100 | 6289 | 50 | | |
| 6198 | 50 | 6290 | 100 | | |
| 6204 | 100 | 6291 | 20 | | |
| 6205 | 100 | 6294 | 60 | | |
| 6207 | 100 | 6302 | 15 | | |
| 6208 | 100 | 6303 | 95 | | |
| 6238 | 90 | 6316 | 25 | | |
| 6239 | 90 | 6320 | 20 | | |

### EXAMPLE 11

### Herbicide Tolerance Conferred by Heterologous Mutant BX Glucosyltransferase Enzymes Expressed in Tobacco

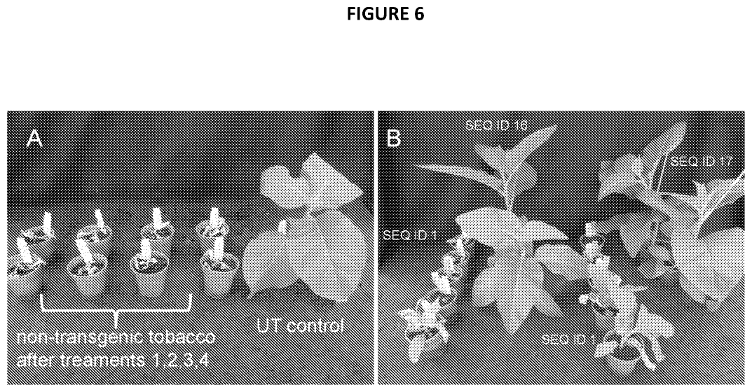

In a further example, Zea mays BX8 or BX9 or orthologues of BX8/9, are altered to carry amino acid variants at various positions which increase tolerance to the alcohol and aminal PSII herbicides as described in the example above. DNA sequences that encode these polypeptides (optimized for tobacco or, optionally, codon optimized according to a target crop such as soybean) were prepared for tobacco transformation as described in example 10. SEQ ID NO: 17 is a variant of SEQ ID NO: 1 and encodes the zmBX9 sequence carrying the S117V, M279F and A334K mutations. SEQ ID NO: 16 is a variant of SEQ ID NO: 1 and encodes the zmBX9 sequence carrying the S117V, M279F and A334R mutations. Transgenic tobacco populations expressing SEQ ID NOs 16 and 17 were generated alongside a population expressing the parental zmBX9 sequence (SEQ ID 1). These populations were sprayed with herbicide V and VI at rates of 200 and 500 g/ha. Plants were assessed for damage and scored at 14 days after treatment (DAT). The results are depicted in Table 19 and also in FIG. 6. It is clear that in comparison to the zmBX9 (SEQ ID NO: 1) tobacco population, several transgenic events from the two variant populations expressing SEQ ID NOs 16 and 17 demonstrate much superior tolerance to both herbicides V and VI as compared to either the w/t non transgenic plants or the transgenic plants expressing only the w/t bx9 glucosyl transferase SEQ ID NO: 1. For example events 7937, 7940, 7952, 8039, 8071 and 8106 expressing SEQ ID NOs 16 and 17 were substantially fully tolerant even at 1 kg/ha of compound VI (data not shown) whereas plants from even the best two events expressing SEQ ID NO: 1 expressed only partial tolerance at high rates.

In FIG. 6 treatments 1, 2, 3 and 4 were 500 g/ha herbicide VI, 1 kg/ha herbicide VI, 200 g/ha herbicide V and 500 g/ha herbicide V. FIG. 6A depicts 4 pairs of non-transgenic tobacco 14 DAT with treatments 1 to 4 (from left to right) adjacent to an untreated control plant. FIG. 6B depicts plants 14 DAT with 500 g/ha of herbicide VI. From left to right the plants in B are 5 clonal plants from a transgenic line of tobacco transformed to express SEQ ID No 1, two plants (separate events) transformed to express SEQ ID NO: 16, 5 clonal plants from another transgenic line of tobacco transformed to express SEQ ID NO: 1 and finally two plants (separate events) transformed to express polypeptide SEQ ID NO: 17.

TABLE 19
GH evaluation of percent damage to tobacco plant lines expressing mutant forms of
Zea mays bx9 glucosyl transferase after treatment with different herbicides

| SED ID | SEQ ID NO 16 | | | SED ID | SEQ ID NO 17 | | | SED ID NO 1 | SEQ ID NO 1 | | | | WT | | |
|---|---|---|---|---|---|---|---|---|---|---|---|---|---|---|---|
| NO32 line | 500 | 200 | 500 | NO33 line | 500 | 200 | 500 | line number | 500 | 200 | 500 | | 500 | 200 | 500 |
| number | gai/ha VI | gai/ha V | gai/ha V | number | gai/ha VI | gai/ha V | gai/ha V | and plant | gai/ha VI | gai/ha V | gai/ha V | WT plant | gai/ha VI | gai/ha V | gai/ha V |
| 7937 | 0 | 0 | 0 | 8022 | 0 | 0 | 5 | 6266 | | | | 1 | 100 | 100 | 100 |
| 7938 | 5 | 100 | 100 | 8026 | 30 | 100 | 100 | A | 80 | 100 | 100 | 2 | 100 | 100 | 100 |
| 7939 | 0 | 5 | 10 | 8029 | 100 | 100 | 100 | B | 95 | 95 | 100 | 3 | 100 | 100 | 100 |
| 7940 | 0 | 0 | 0 | 8039 | 0 | 0 | 0 | C | 90 | 25 | 100 | 4 | 100 | 100 | 100 |
| 7944 | 0 | 100 | 100 | 8040 | 1 | 1 | 55 | D | 95 | 100 | 100 | 5 | 100 | 100 | 100 |
| 7945 | 1 | 10 | 100 | 8042 | 1 | 100 | 100 | E | 100 | 100 | 100 | 6 | 100 | 100 | 100 |
| 7946 | 0 | 0 | 1 | 8046 | 5 | 20 | 50 | 6291 | | | | 7 | 100 | 100 | 100 |
| 7951 | 35 | 100 | 100 | 8050 | 1 | 60 | 100 | A | 60 | 80 | 100 | 8 | 100 | 100 | 100 |
| 7952 | 0 | 0 | 0 | 8051 | 1 | 10 | 15 | B | 70 | 70 | 80 | 9 | 100 | 100 | 100 |
| 7953 | 0 | 0 | 0 | 8052 | 5 | 100 | 100 | C | 75 | 80 | 100 | | | | |
| 7956 | 1 | 100 | 100 | 8053 | 1 | 30 | 100 | D | 80 | 100 | 100 | | | | |
| 7957 | 10 | 100 | 100 | 8056 | 1 | 65 | 100 | E | 100 | 100 | 100 | | | | |
| 7959 | 0 | 0 | 1 | 8058 | 0 | 100 | 100 | | | | | | | | |
| 7960 | 0 | 0 | 1 | 8059 | 0 | 100 | 100 | | | | | | | | |
| 7962 | 0 | 100 | 100 | 8061 | 95 | 100 | 100 | | | | | | | | |
| 7970 | 1 | 100 | 100 | 8062 | 1 | 55 | 60 | | | | | | | | |
| 7971 | 1 | 1 | 30 | 8064 | 0 | 100 | 100 | | | | | | | | |
| 7975 | 0 | 0 | 1 | 8065 | 0 | 0 | 15 | | | | | | | | |
| 7977 | 0 | 5 | 1 | 8067 | 0 | 5 | 80 | | | | | | | | |
| 7979 | 0 | 1 | 1 | 8069 | 0 | 50 | 20 | | | | | | | | |
| 7980 | 0 | 100 | 100 | 8070 | 0 | 5 | 100 | | | | | | | | |
| 7982 | 1 | 100 | 55 | 8071 | 0 | 0 | 0 | | | | | | | | |
| 7985 | 5 | 100 | 100 | 8076 | 10 | 0 | 0 | | | | | | | | |
| 7986 | 0 | 1 | 1 | 8077 | 10 | 1 | 1 | | | | | | | | |
| 7989 | 0 | 1 | 5 | 8080 | 0 | 0 | 5 | | | | | | | | |
| 7990 | 0 | 1 | 0 | 8081 | 0 | 0 | 5 | | | | | | | | |
| 8007 | 100 | 100 | 100 | 8093 | 0 | 1 | 5 | | | | | | | | |
| 8011 | 100 | 100 | 100 | 8094 | 1 | 0 | 5 | | | | | | | | |
| 8012 | 20 | 1 | 5 | 8095 | 0 | 0 | 30 | | | | | | | | |
| 8013 | 0 | 1 | 5 | 8106 | 0 | 0 | 0 | | | | | | | | |

### EXAMPLE 12

### Production and Characterization of Beta-Glucosides of Compounds V and VI Monitored by LC MS

The enzyme product glucosides of herbicides V and VI are formed by carrying out enzyme assay reactions as described in Example 1. 50 or 100 μl samples from assay reactions carried out as described in example 1 are added to 500 μl ethyl acetate to stop the reaction. Samples are vortexed and 400 μl of the ethyl acetate partition removed, dried down, and resuspended in 100 μl 80:20 acetonitrile/water. Samples are transferred to vials and analyzed by LC-MS using an Agilent 1290 liquid chromatography system and Thermo Q-Exactive mass spectrometer. Chromatography is achieved on a Waters Atlantis dC18 (100×2.1 mm) 5 μm particle size column or a Waters Acquity C18 BEH (50×2.1 mm) 1.7 μm particle size column, using a 12 or 6 minute gradient run of Water (0.2% formic acid) and Acetonitrile. The Q-Exactive is operated in positive ionisation electrospray mode, using Full scan-AIF mode, at 35,000 resolution, between 100-800 m/z. All analytes are identified from the full scan data to within at least 5 ppm accuracy of their predicted pseudo-molecular ion [M+H]⁺ m/z value.

In order to unambiguously identify the particular glucosides of herbicides V and VI that are made in the enzyme reactions the various possible glucosides are made synthetically as standards in order to characterize the LC/MS profile of each. Firstly herbicides V and VI are synthesized as described in the patents and patent applications included infra. The various glucoside derivatives used as standards for LCMS are then synthesized, separated and characterized as described below. These standards resulting from synthesis and chromatography were designated as follows.
22902-11: O-glucoside (mixture of two stereoisomers (not separated) of compound V. Major component was the α-glucoside and the minor component was the β-glucoside.
22902-12: O-β-glucoside of compound V. A resolved pure stereoisomer, either R or S-beta but the opposite of 22902-13
22902-13: O-β-glucoside of compound V. A resolved pure stereoisomer, either R or S-beta but the opposite of 22902-12
22902-14: O-(S-α)-glucoside of compound VI
22902-15: O-(S-β)-glucoside of compound VI

The glucosides of V are made in 2 steps from V. The first is reaction of V with an excess of tetra acetate protected alpha glucosyl bromide, activated with mercury (II) oxide and catalytic mercury (II) bromide. This yields a mixture of the 4 isomers which are not separated at this stage. In the second step global acetate deprotection is performed using catalytic sodium methoxide in methanol, and the isomers S beta, S alpha, R beta and R alpha are separated using preparative chiral liquid chromatography. 1 equivalent (eq)=1.00 g of V is dissolved in 10 ml dichloromethane (DCM), cooled to 0° C. then 4 eq=6.30 g [(2R,3R,4S,5R,6R)-3,4,5-triacetoxy-6-bromo-tetrahydropyran-2-yl]methyl acetate is added. Then 2 g of 4 A molecular sieves (freshly dried at 200 degC 20 mBar) was added, then 1.1 eq=912 mg yellow HgO and 0.05 eq=69 mg HgBr₂ are then added and the ice bath is removed and the mixture allowed to warm to room temperature with stirring. The reaction mixture is stirred at room temperature for 16 hours and then heated to 50° C. for 10 mins, then heated to 60° C. for 30 minutes, to a point at which LCMS analysis indicates that all of the V is consumed and that there are 4 LC peaks formed in about 7% total yield. The reaction is further worked up by diluting with 70 ml DCM then washing with 30 ml water, the water is back extracted with 10 ml DCM, and the combined DCM solution dried with Na₂SO4, filtered and evaporated under vacuum to give about 7.3 g of a yellow foam product. Isomer separation by normal phase and reverse phase chromatography is difficult so fractions are combined fractions to yield about 300 mg of white solid. LCMS (pos ES) confirms this to be a mixture of all 4 isomers with MH+ 592. This mixture is then deprotected. In this deprotection step, 1 eq=110 mg of the 4 acetate protected isomer mixture is dissolved in 2 ml dry MeOH then 0.1 eq=38 ul NaOMe (0.5M in MeOH) is added and the reaction stirred at room temperature for 50 mins, at which point LCMS analysis confirms that full deprotection of the acetates has occurred. At this point the reaction is neutralised carefully by cooling to 0° C. and adding 11 ul HCl (2M aq) in 2 ul portions to a final pH of 5-6. The bulk of MeOH is evaporated under vacuum at room temperature and the residue purified by reverse phase chromatography to give about 33 mg gum. LCMS and chiral LC confirmed this to be a mix of the 4 isomers. This mixture was purified using preparative chiral LC to give 3 samples called 22902-11, 22902-12 and 22902-13 designated as above.

Sample 22902-11 was produced in a yield of 7 mg. Proton NMR indicated that there were 2 glucosides, the major component with alpha stereochemistry at the anomeric position and the minor with beta stereochemistry at the anomeric position. LCMS (positive ion mode ES) showed MH+ 424.

Sample 22902-12 was produced in a yield of 8 mg. NMR indicated that >95% of the 1-glucoside had alpha stereochemistry at the anomeric position and LCMS (pos ES) again confirmed a MH+ of 424.

Sample 22902-13 was produced in a yield of 10 mg. NMR indicated showed >95% of 1-glucoside with beta stereochemistry at the anomeric position. LCMS (pos ES) again showed MH+ 424.

The glucosides of VI are made in 2 steps from VI. The first step is reaction of VI with an excess of tetra acetate protected alpha glucosyl bromide, activating with silver (I) triflate. This yields a mixture of the 2 isomers (below) which are not separated at this stage. In the second step the acetates are removed using catalytic sodium methoxide in methanol, and the 2 isomers separated using preparative reverse phase LC and MS detection.

1 eq=1.00 g of VI is dissolved in 10 ml DCM, cooled to 0° C. then 4 eq=5.65 g [(2R, 3R, 4S, 5R, 6R)-3,4,5-triacetoxy-6-bromo-tetrahydropyran-2-yl]methyl acetate is added. Then 1 g of 4 A molecular sieves is added and then 4 eq=3.55 g silver (I) triflate is added and the reaction mixture stirred at 0° C. for 20 hours to a point at which point LCMS shows that the 2 isomers are formed. The reaction is then worked up by filtering through celite under vacuum, washing with DCM and evaporating the filtrate under vacuum to give about 7.5 g of black gum. This residue is purified by normal phase then reverse phase chromatography to give about 294 mg of white solid. NMR showed this to be a mixture of 2 glucosides, the major component was alpha stereochemistry at the anomeric position, the minor component was beta stereochemistry at the anomeric position. LCMS (pos ES) showed MH+ 622. This mixture is then deprotected. 1 eq=110 mg of the mixture of the 2 acetate protected isomers was dissolved in 2 ml dry MeOH then 0.05 eq=18 ul NaOMe (0.5M in MeOH) is added and the reaction stirred at room temperature for 80 minutes, at which point LCMS showed full deprotection of the acetates. The reaction is neutralised carefully by cooling to 0° C. and adding 11 ul HCl (2M aq) in 2 ul portions, the final pH was measured at 7. The sample was purified by reverse phase chromatography to give 2 samples called 22902-14: and 22902-15.

Sample 22902-14: was produced in a yield of 58 mg. NMR indicated the sample to be >95% 1 glucoside with alpha stereochemistry at the anomeric position, and anti-stereochemistry in the 5 membered ring. LCMS (pos ES) showed MH+ 454.

Sample 22902-14: was produced in a yield of 0.7 mg. NMR indicated the sample to be 90% 1 glucoside with beta stereochemistry at the anomeric position, and anti-stereochemistry in the 5 membered ring. LCMS (pos ES) showed MH+ 454.

Note that the glucosides were assigned as alpha or beta according to the NMR coupling constant to the anomeric carbon.

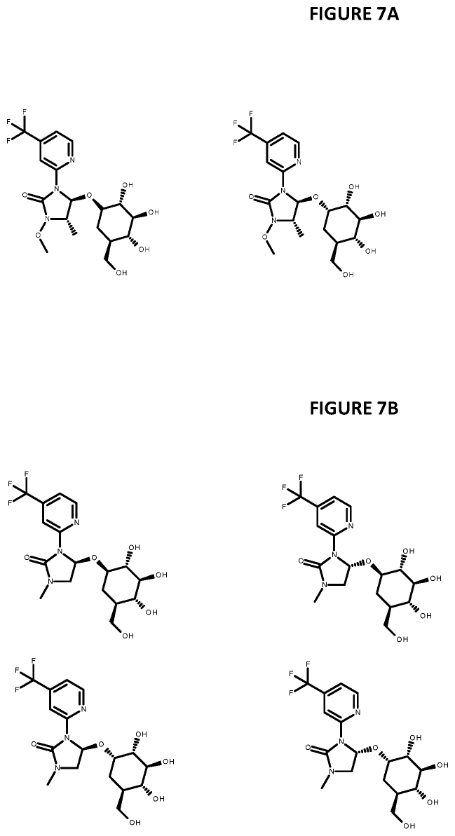

All standards were made up to 0.5 μM in 80:20 acetonitrile/water for analysis. The structures of the various glucosides of herbicide V and VI are depicted in FIGS. 7A and 7B.

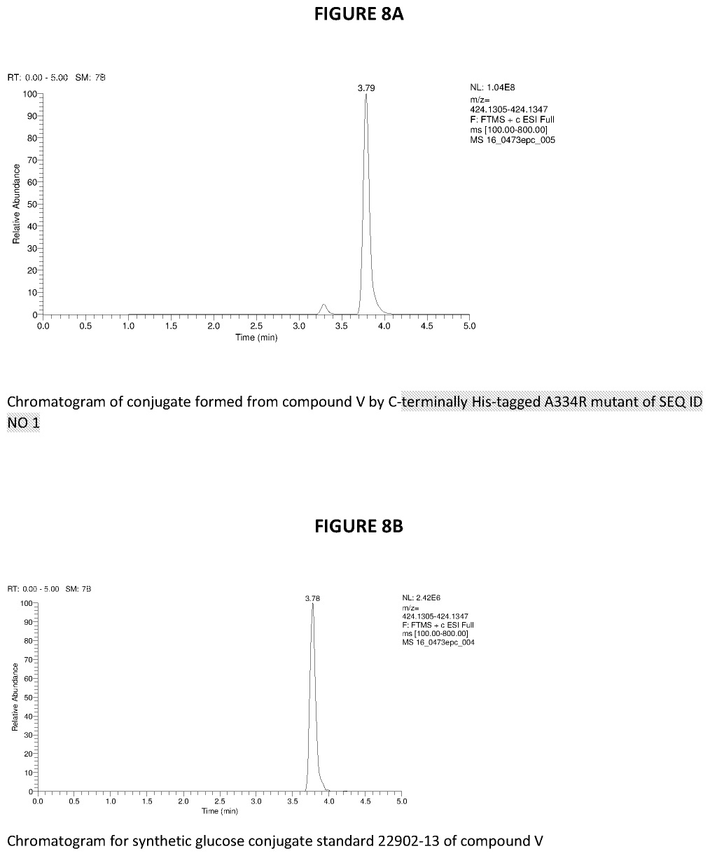

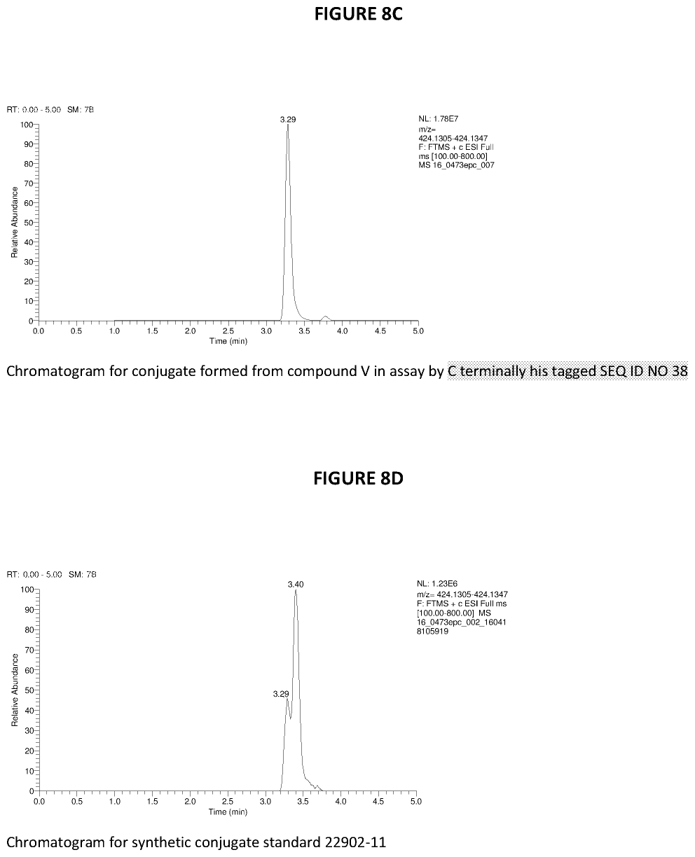

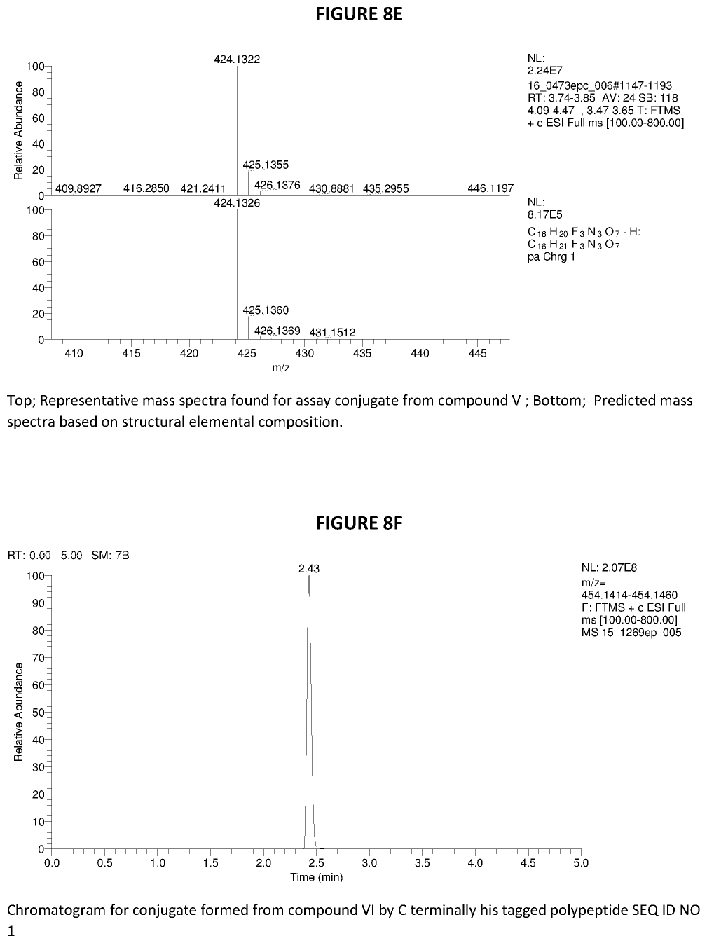

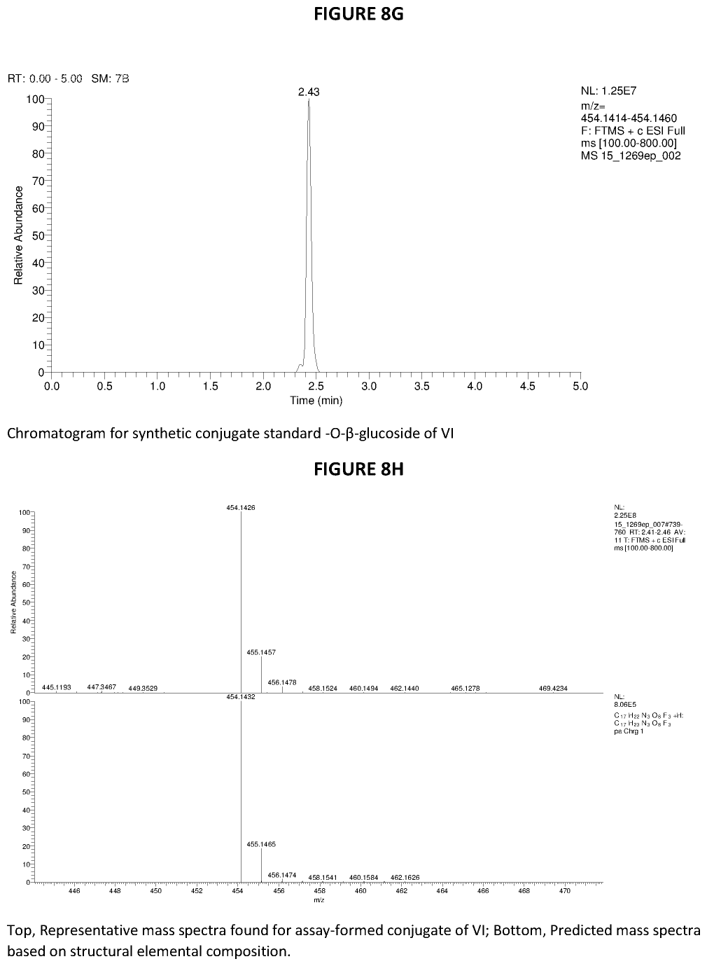

The different stereoisomer standards chromatographed distinctly on LC (FIGS. 8A-8H). By comparing the LC elution profiles of the standards with the enzyme products at the correct mass it was possible to characterize which polypeptides catalyzed the formation of which O-glucosides. Assay samples and glucoside conjugate standards are run using the identical chromatographic conditions to allow us to identify the conjugate isomer formed in the assay. In short, as would be expected for inverting glucosyl transferases all of the bx enzymes tested produced beta glucoside products (from the alpha-UDP-glucose substrate) from all herbicides tested. But there can be subtle variations between enzymes in stereochemistry of the products made at other chiral centers. For example, in the case of compound V, the C-terminally His tagged SEQ ID NO1 and the similarly C-terminally His tagged derivative of the A334R mutant of SEQ ID NO1 both catalyzed formation of predominantly the conjugate product matching the 22902-13 isomer standard, whereas the assay similarly run with the C-terminally His-tagged Zea mays BX8 derivatives of SEQ ID's 38 and 51 (Bx8 V367I+H376C double mutation and bx8/bx9 hybrid 2) both gave predominately a conjugate product that was distinct from 22902-13 and which matched the minor component of the 22902-11 isomer standard. Therefore all of these derivatives of Zea mays BX8 and BX9 produced a -β-glucoside conjugate (following the α-inversion glucosylation mechanism) but, for compound V, with BX8 and BX9 each mainly producing the opposite stereochemistry (R and S-configuration beta glucosides) at the dihydrohydantoin ring. It was estimated (crude extract assays only) from the LC MS peaks that the bx8 sequences SEQ ID No 40 and 50 exhibited up to about half the activity of the w/t bx9 sequence in respect of compound V.

In the case of structure VI all enzymes tested produced only the S-beta stereoisomer O-glucoside product. This was confirmed by comparison of the LC chromatography with the two glucoside standards of compound VI (FIGS. 8A-8H).

The expected glucosides of herbicides V and VI corresponding to those seen in vitro are also similarly produced in transgenic and non-transgenic plants expressing Zea mays BX9 or expressing mutant derivatives of Zea mays BX9. For example, LC/MS analysis of extracts of leaves obtained by maceration and extraction into 80% acetonitrile/water 24 and 48 h after treatment with herbicides V and VI indicate that the same beta-glucosides that are produced by the enzymes in vitro are produced in planta. Thus, such acetonitrile foliar extracts of VI-treated non-transgenic w/t Zea mays seedlings (i.e. Zea mays naturally expressing bx9 and bx8) are found to comprise not only parent herbicide VI but also the S-beta stereoisomer O-glucoside product of VI. Similarly, the glucosides in extracts of herbicide VI-treated transgenic tobacco plants expressing for example SEQ ID NO1 or SEQ ID NO 17 (see example 10 and example 11) are found also to comprise mainly the S-beta stereoisomer O-glucoside product of VI (and in higher amounts according to the expression level and increased activity level of the SEQ ID No 17 mutant bx glucosyl transferase polypeptide relative to the w/t, SEQ ID No 1 versus herbicide VI).

### EXAMPLE 13

### Homology-Dependent Sequence Replacement Using CRISPR Cas9 System

Using CRISPR-Cas9 the NP2222 maize endogenous bx9 gene was replaced with a donor harboring 6 amino acid mutations as compared to the wild-type genome sequence. To achieve this goal, CRISPR-Cas9 vectors were designed to make double stranded breaks (DSB) at specific site in the bx9 gene. Donor DNA was provided as a template while double stranded breaks were made at the specific genome locations to facilitate homology dependent repair. To study the length effect of homology arms on targeted gene replacement, CRISPR Cas9 expression vectors were constructed and targeted replacement experiments were performed using biolistic bombardment delivery. Taqman assays were used to detect mutations in the target site and overlapping junction PCRs were performed to identify plants containing the targeted gene replacement.

### Construction of Vectors for Cas9 and Donor Vectors for Targeted Gene Replacement in Maize

Construction of Cas9 expression vectors and targeting donors have been described before (WO16106121, incorporated by reference herein). The maize-optimized Type II Cas9 gene from Streptococcus pyogenes SF370 (cBCas9Nu-01) was driven under the control of a sugarcane ubiquitin promoter by NOS terminator for CRISPR Cas9 vector 23935. A nuclear localization signal was also incorporated into the C-terminus of Cas9 to improve its targeting to nucleus.

Two target sequences (5′-acttgccaattgccatatag-3′ SEQ ID No. 136, 5′-aatcctcgctcgctcacgct-3′ SEQ ID No. 137) were selected to target at the left end of bx9 gene and two (5′-ccgcacggatttaaccgatt-3′ SEQ ID No. 138, 5′-acacaacaccgtcaggaacg-3′ SEQ ID No. 139) at the right end of bx9 gene. Vector 23935 expresses one PMI cassette as selectable marker, one Cas9 expression cassette to introduce DSBs in the targeted loci, and four single gRNAs that can guide Cas9-mediated cleavage of maize genomic sequence ZmBx9V1 (SEQ ID No. 136), ZmBx9V2 (SEQ ID No. 137), ZmBx9V3 (SEQ ID No. 138), and ZmBx9V4 (SEQ ID No. 139), located within the Bx9 locus in elite maize variety NP2222. The sgRNA expression cassettes are comprised of either rice U3 promoter (prOsU3) or rice U6 promoter (prOsU6), and coding sequences for each of their sgRNAs named sgRNAZmBx9V1(SEQ ID No. 140), sgRNAZmBx9V2 (SEQ ID No. 141), sgRNAZmBx9V3(SEQ ID No. 142), and sgRNAZmBx9V4(SEQ ID No. 143), respectively.

sgRNAZmBx9V1 is comprised of the 20-nt specificity-conferring targeting RNA xZmBx9V1 fused with the crRNA and tracrRNA scaffold sequences for interaction with Cas9 (SEQ ID No. 144). sgRNAZmBx9V2 is comprised of the 20-nt specificity-conferring targeting RNA xZmBx9V2 fused with the crRNA and tracrRNA scaffold sequences for interaction with Cas9 (SEQ ID No. 145). sgRNAZmBx9V3 is comprised of the 20-nt specificity-conferring targeting RNA xZmBx9V3 fused with the crRNA and tracrRNA scaffold sequences for interaction with Cas9 (SEQ ID No. 146). sgRNAZmBx9V4 is comprised of the 20-nt specificity-conferring targeting RNA xZmBx9V4 fused with the crRNA and tracrRNA scaffold sequences for interaction with Cas9 (SEQ ID No. 147).

The expression cassettes comprising prOsU3 promoter/prOsU6 promoter and sgRNAZmBx9V5-V8 (SEQ ID Nos. 144-147) were cloned into a biolistic transformation vector along with the Cas9 expression cassette to form 23935 (FIG. 1).

Donor vector 23939 was designed to include a 1666 bp DNA sequence containing a 48 bp change from wild type genomic sequence (xB73Bx9 SEQ ID No. 148), flanked by 1584 bp and 1424 bp arms homologous to genomic target locus (xJHAXBx9-01 SEQ ID No. 149 and xJHAXBx9-02 SEQ ID No.150) (FIG. 2).

Donor fragment 23939A is a 3.1 kb DNA fragment produced from San DI and SbfI enzyme digestion of 23939. 23939A features a 1666 bp DNA fragment containing desired genome sequence in the middle (xB73Bx9 SEQ ID No. 148) to replace the wide type Bx9 gene flanked by 52 bp and 1428 bp arms homologous to the genomic target locus (xJHAXBx9-01 SEQ ID No. 151 and xJHAXBx9-02 SEQ ID No. 152) 5′ and 3′ to the cassettes, respectively homologous to the bx9 region of NP2222 maize genome (FIG. 3A).

Donor fragment 23939B is an 1.9 kb high fidelity PCR amplification product using 23939 as template, AZ15 serves as forward primer (5′-AATGGACCACCCGACCGTGTC-3′), and AZ16 (5′-GCACAATGGTACACCAAGAACAC-3′) as reverse primer. 23939B features a DNA fragment containing desired genome sequence in the middle to replace the wide type bx9 gene sequence, flanked by 121 bp and 111 bp arms homologous to the genomic target locus (xJHAXBx9-01SEQ ID No. 153 and xJHAXBx9-02 SEQ ID No. 154) 5′ and 3′ to the cassettes, respectively homologous to the Bx9 locus of NP2222 maize genome (FIG. 3B).

The sequences of homology arms are identical to part of the bx9 gene sequences and are used for guiding the targeted allele replacement of the donor sequences to the Cas9 cleavage site at the target locus using homologous recombination.

### Generation of Targeted Gene Replacement Mutant Using Biolistic Bombardment

To generate potential mutants carrying the desired sequence replacing wild type Bx9 gene, elite maize transformation variety NP2222 was chosen for all experiments as described (U.S. Pat. No. 9,133,474 and WO16106121, incorporated by reference herein). CRISPR vector 23935 and donor 23939A or 23939B were co-delivered to maize immature embryos through biolistic transformation (FIG. 3?). Methods for maize immature embryo bombardment, callus induction and selection, plant regeneration and rooting have been described previously (Wright et al., 2001, Plant Cell Reports 20:429-436). Briefly, immature embryos were isolated from sterilized immature ears of elite maize variety NP2222 at 9-11 days after pollination, and pre-cultured for 1 to 3 days on osmoticum media. Plasmid DNA of a vector 23935, carrying an expression cassette for Cas9Nuc and sgRNAs, was mixed with a donor fragment from vector 23939 which comprises the desired Bx9 sequence and homology arms. The DNA mixture was then co-precipitated onto gold particles and used to bombard pre-cultured embryos. After bombardment with the DNA-gold particles using BioRad PDS-1000 Biolistic particle delivery system as described, bombarded embryos were then incubated in callus induction media and then moved onto mannose selection media. Mannose resistant calli were selected to regeneration media for shoot formation. Shoots were then sub-cultured onto rooting media. Samples were then harvested from rooted plants for Taqman assays to detect mutations in the target site and overlapping junction PCRs were performed to identify potential plants containing the targeted gene replacement.

Table 1 shows an experiment comparing different donor sizes with the same CRIPSR cas9 vector 23935. Donor 23939A is 3.1 kb with 52 bp and 1428 bp arms homologous to the NP2222 maize genome, while 23939B is 1.9 kb in size with 121 bp and 111 bp homology arms. Data showed that there is no significant difference in obtaining targeted gene replacement between treatment A and B. 8.2% of plants analyzed for treatment A are positive for either 5′ or 3′ end junction PCR, while 8.9% for treatment B showed positive band for junction PCR in at least one end of the target gene. 1.72% verse 1.48% of analyzed lines are both end junction PCR positive for treatment A and B, respectively. This data suggests a minimum of ˜100 bp homology arms for successful large gene fragment replacement. The homology dependent repair efficiency appears not be affected when using smaller size of homology arms.

TABLE 20
Targeted allele replacement with different donor size

| | | | Donor | Size L & | Immature | | | | either | % of | Both | % of |
|---|---|---|---|---|---|---|---|---|---|---|---|---|
| Treat- | CRISPR | | size | R Arms | embryo | PMI + | | Events | 5′ or 3′ | either | 5′&3′ | both |
| ment | vector | Donor | (Kb) | (bp) | targets | plants | TF % | in GH | PCR+ | PCR+ | PCR+ | PCR+ |
| A | 23935 | 23939 A | 3.1 | 52, 1428 | 4404 | 299 | 6.80% | 232 | 19 | 8.20% | 4 | 1.72% |
| B | 23935 | 23939 B | 1.9 | 121, 111 | 1427 | 121 | 8.50% | 135 | 12 | 8.90% | 2 | 1.48% |

### EXAMPLE 14

### Enhanced Homology-Dependent Sequence Replacement with Single Cleavage at the Target Site Using CRISPR-cas9 System

To test the minimum size of homology arms needed for large gene replacement, donor vector 23984 was designed to include a 1116 bp DNA sequence containing 13 bp change from wild type genomic sequence in the middle (cZmUGTBx9 SEQ ID No. 155), flanked by 49 bp and 40 bp arms homologous to genomic target locus (xJHAXBx9 SEQ ID No. 156 and cZmUGTBx9 SEQ ID No. 157) (FIG. 4).

Donor fragment 23984A is a 1.2 kb high fidelity PCR amplification product using 23984 as a template, SD53 as forward primer (5′-CTGTCCGTCCGCTTCTCTCTCCC

-3′) (SEQ ID NO: 187) and SD54 (5′-GCTTGGCCTGCAGGCGACGG-3′) (SEQ ID NO. 188) as reverse primer.

To compare the impact of single cleavage site and double cleavage sites for efficient large gene replacement, CRISPR cas9 vector 23792 harboring one single gRNA and 24001 harboring two single gRNAs which will make two cleavages in the target gene, were constructed (FIGS. 5 and 6). Both 23792 and 24001 contain exactly the same Cas9 expression cassette for cleavage and PMI cassette for tissue culture selection.

To design CRISPR cas9 vector 23792, one target sequence ZmBx9-M279F (5′-gtacgtcagcttcggga/gcaTGG-3′ SEQ ID No. 158) was chosen for testing Cas9-gRNA mediated gene replacement. 23792 expresses a sgRNA that can guide Cas9-mediated cleavage of maize genomic sequence ZmBx9-M279F (SEQ ID No. 158). The sgRNA expression cassette is comprised of rice U3 promoter (prOsU3), and coding sequences for sgRNA named sgRNAZmBx9-M279F (SEQ ID No. 159). sgRNAZmBx9-M279F is comprised of the 20-nt specificity-conferring targeting RNA xZmBx9-M279F (SEQ ID No. 160) fused with the crRNA and tracrRNA scaffold sequences for interaction with Cas9. The expression cassettes comprising prOsU3 promoter and sgRNAZmBx9-02 (SEQ ID No. 160) were cloned into a biolistic transformation vector along with the Cas9 expression cassette to form 23792 (FIG. 5).

To create CRISPR cas9 vector 24001, two target sequence ZmBx9-A334K (5′-gccgcggcatcgtcgtc/accTGG-3′ SEQ ID No. 161) and ZmBx9V2 target (5′-aatcctcgctcgctcac/gctCGG-3′ SEQ ID No. 162) were chosen for testing Cas9-gRNA mediated gene replacement. 24001 expresses two sgRNAs that can guide Cas9-mediated cleavage of maize genomic sequence ZmBx9-A334K (SEQ ID No. 161) and ZmBx9V2 (SEQ ID No. 162). The sgRNA expression cassette is comprised of rice U3/U6 promoter (prOsU3/U6), and coding sequences for sgRNAs named sgRNAZmBx9-03 (SEQ ID No. 163) and sgRNAZmBx9-05 (SEQ ID No. 164), respectively. sgRNAZmBx9-03 is comprised of the 20-nt specificity—conferring targeting RNA xZmBx9-03 (SEQ ID No. 165) fused with the crRNA and tracrRNA scaffold sequences for interaction with Cas9. sgRNAZmBx9-05 is comprised of the 20-nt specificity—conferring targeting RNA xZmBx9-05 (SEQ ID No. 166) fused with the crRNA and tracrRNA scaffold sequences for interaction with Cas9. The expression cassettes comprising prOsU3/U6 promoter and sgRNAZmBx9-03 (SEQ ID No. 165)/sgRNAZmBx9-05 (SEQ ID No. 166) were cloned into a biolistic transformation vector along with the Cas9 expression cassette to form 24001 (FIG. 6).

To generate potential mutants carrying desired sequence replacing wild type bx9 gene, elite maize transformation variety NP2222 was chosen for all experiments as described in example 1. Vector 23792 or 24001 and donor 23984A were co-delivered to maize immature embryos.

Table 21 shows a study to compare the impact of single cleavage site and double cleavage sites for large gene replacement efficiency. Donor 23984A was used for both treatment A and B. For treatment A, CRISPR cas9 vector 23792 which was designed to cleave at a location within the target gene was co-delivered with donor 23984A. For treatment B, CRISPR Cas9 vector 24001 which was designed to cleave on the 5′ and 3′ end of target region was co-delivered with donor 23984A.

Junction PCR data showed that treatment A had 20 out of 262 (7.6%) tested plants showing at least one end PCR positive, while treatment B showed 11.78% of tested plants are positive on at least one end of junction PCR, indicating successful gene replacement from at least one end of the target gene. It appears that CRISPR vector 24001 with two single gRNA might work more efficiently for targeted large gene replacement, which is not the case for a small change in the genome. One single gRNA is commonly used for small allele replacement. However, 9 out of 262 (3.44%) tested plants from treatment A showed expected size of band on electrophoresis gel for both end junction PCR, while only 3 out of 294 tested plants showed expected size of band for both end junction PCR with treatment B. It indicates that significantly higher gene replacement efficiency may be obtained when CRISPR vector cleaves only once on the target gene.

TABLE 21
Targeted allele replacement efficiency comparison with single or double cleavage

| | | | Donor | Size L & | Immature | | | | either | % of | Both | % of |
|---|---|---|---|---|---|---|---|---|---|---|---|---|
| Treat- | CRISPR | | size | R Arms | embryo | PMI + | | Events | 5′ or 3′ | either | 5′&3′ | both |
| ment | vector | Donor | (kB) | (bp) | targets | plants | TF % | in GH | PCR+ | PCR+ | PCR+ | PCR+ |
| A | 23792 | 23984 A | 1.2 | 49, 40 | 6666 | 334 | 5.01% | 262 | 20 | 7.60% | 9 | 3.44% |
| B | 24001 | 23984 A | 1.2 | 49, 40 | 8459 | 247 | 2.92% | 297 | 35 | 11.78% | 3 | 1.01% |

### EXAMPLE 15

### Homology-Dependent Sequence Replacement Using CRISPR-Cpf1 System

Cpf1 (cLbCpf1-02) is an RNA-guided endonuclease of a class II CRISPR system. CRISPR/Cpf1 stands for Clustered Regularly Interspaced Short Palindromic Repeats from Prevotella and Francisella 1. Cpf1 create staggered end which has great potential to enhance precise gene replacement using non-homologous end joining (NHEJ).

Using CRISPR-Cpf1 the NP2222 maize endogenous bx9 gene was replaced with a donor harboring 6 amino acid mutations as compared to WT genome sequence. To achieve this goal, we designed CRISPR-Cpf1 vectors to make double stranded break at specific site in the Bx9 gene. Donor DNA were provided as template while DSB was introduced at the specific genome locations to facilitate homology directed repair.

### Construction of Vectors for Cpf1 and Donor Vectors for Targeted Gene Replacement in Maize

The Cpf1 used in this example is a rice codon-optimized version from Lachnospiraceae bacterium ND2006 (Tang et al., 2017), with 3 bp changes to remove 2 Bsp119I and one RsrII sites. Two nuclear localization signals (NLS) are added at its N- and C-terminals respectively; N terminus also contains an epitope tag. cLbCpf1-02 was driven under the control of a sugarcane ubiquitin promoter followed by NOS terminator for CRISPR cpf1 vectors. Four CRISPR Cpf1 vectors and one donor vector were made for this study.

To design CRISPR Cpf1 vector 24096, one target sequence (5′-TTTC/accgg/caggtagcccttgtcgat-3′ SEQ ID No. 167), was selected to target in the middle of the bx9 gene. Vector 24096 express 1 PMI cassette as selectable marker, 1 Cpf1 expression cassette to introduce staggered DSB in the targeted loci, and 1 crRNA that can guide Cpf1-mediated cleavage of maize genomic sequence ZmBx9 Target3r (SEQ ID No. 167), located within the bx9 locus in elite maize variety NP2222. The crRNA expression cassette is comprised of sugarcane ubiquitin-4 promoter (prSoUbi4-02), and coding sequence (SEQ ID No. 168) named rLbgRNACpf1ZmUGTBx9-01. rLbgRNACpf1ZmUGTBx9-01 (SEQ ID No. 169) is comprised of the 23-nt specificity-conferring targeting RNA xZmBx9Target3r fused with the crRNA sequences for interaction with Cpf1. The expression cassette comprising sugarcane ubiquitin promoter and rLbgRNACpf1ZmUGTBx9-01 (SEQ ID No. 169) were cloned into a biolistic transformation vector along with the Cpf1 expression cassette to form 24096 (FIG. 8).

To design CRISPR Cpf1 vector 24098, one target sequence (SEQ ID No. 170), was selected to target at the middle of bx9 gene. Vector 24098 express 1 PMI cassette as selectable marker, 1 Cpf1 expression cassette to introduce staggered DSB in the targeted loci, and 1 crRNA that can guide Cpf1-mediated cleavage of maize genomic sequence ZmBx9 Target4r (SEQ ID No. 170), located within the Bx9 locus in elite maize variety NP2222. The crRNA expression cassette is comprised of sugarcane ubiquitin-4 promoter (prSoUbi4-02), and coding sequence (SEQ ID No. 171) named rLbgRNACpf1ZmUGTBx9-01. rLbgRNACpf1ZmUGTBx9-02 (SEQ ID No. 172) is comprised of the 23-nt specificity-conferring targeting RNA xZmBx9Target4r fused with the crRNA sequences for interaction with Cpf1.

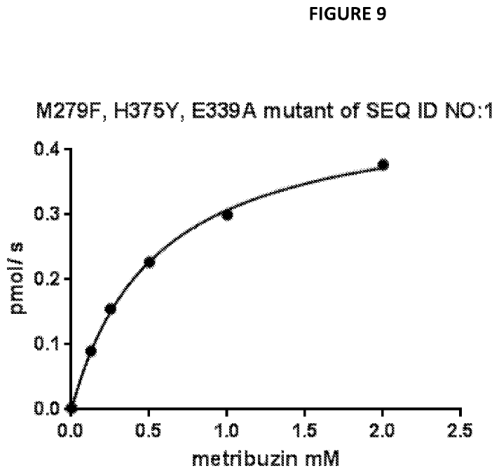

The expression cassette comprising sugarcane ubiquitin promoter and rLbgRNACpf1ZmUGTBx9-02. (SEQ ID No. 172) were cloned into a biolistic transformation vector along with the Cpf1 expression cassette to form 24098 (FIG. 9).

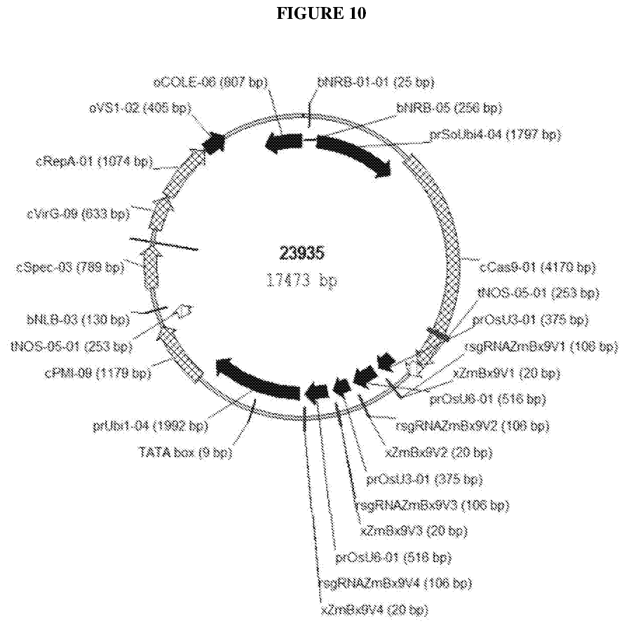

To design CRISPR Cpf1 vector 24099, one target sequence (SEQ ID No. 173), was selected to target at 5′ end of bx9 gene. Vector 24099 express 1 PMI cassette as selectable marker, 1 Cpf1 expression cassette to introduce staggered DSB in the targeted loci, and 1 crRNA that can guide Cpf1-mediated cleavage of maize genomic sequence ZmBx9Target7 (SEQ ID No. 173), located within the bx9 locus in elite maize variety NP2222. The crRNA expression cassette is comprised of sugarcane ubiquitin-4 promoter (prSoUbi4-02), and coding sequence (SEQ ID No. 174) named rLbgRNACpf1ZmUGTBx9-01. rLbgRNACpf1ZmUGTBx9-03 (SEQ ID No. 175) is comprised of the 23-nt specificity-conferring targeting RNA ZmBx9Target7 fused with the crRNA sequences for interaction with Cpf1. The expression cassette comprising sugarcane ubiquitin promoter and rLbgRNACpf1ZmUGTBx9-01. (SEQ ID No. 175) were cloned into a biolistic transformation vector along with the Cpf1 expression cassette to form 24099 (FIG. 10).

To design CRISPR Cpf1 vector 24100, one target sequence (SEQ ID No. 176), was selected to target at the 3′ end of bx9 gene. Vector 24100 express 1 PMI cassette as selectable marker, 1 Cpf1 expression cassette to introduce staggered DSB in the targeted loci, and 1 crRNA that can guide Cpf1-mediated cleavage of maize genomic sequence ZmBx9Target7 (SEQ ID No. 176), located within the bx9 locus in elite maize variety NP2222. The crRNA expression cassette is comprised of sugarcane ubiquitin-4 promoter (prSoUbi4-02), and coding sequence (SEQ ID No. 177) named rLbgRNACpf1ZmUGTBx9-01. rLbgRNACpf1ZmUGTBx9-03 (SEQ ID No. 178) is comprised of the 23-nt specificity-conferring targeting RNA xZmBx9Target2 fused with the crRNA sequences for interaction with Cpf1.

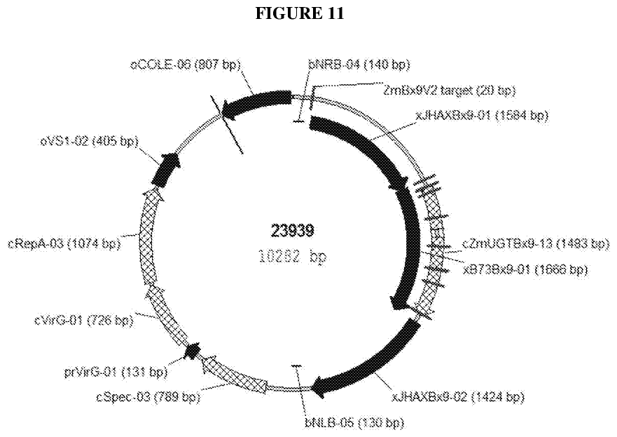

The expression cassette comprising sugarcane ubiquitin promoter and rLbgRNACpf1ZmUGTBx9-01 (SEQ ID No. 178) were cloned into a biolistic transformation vector along with the Cpf1 expression cassette to form 24100 (FIG. 11).

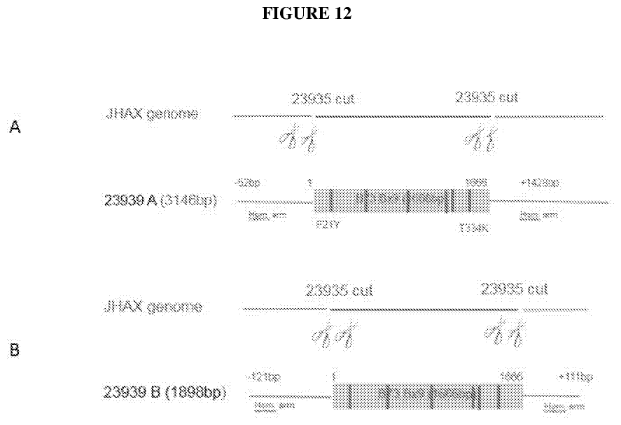

Donor vector 24101 was designed to include ˜1.5 Kb DNA sequence containing 19 bp change from wild type genomic sequence (cZmUGTBx9-17 SEQ ID No. 184), flanked by left and right arms homologous to genomic target locus (xJHAXBx9-05 and xJHAXBx9-02) (FIG. 12).

To test whether a minimum of 35 bp homology arms are sufficient for successful large gene fragment replacement, 3 different donors were created using high fidelity PCR.

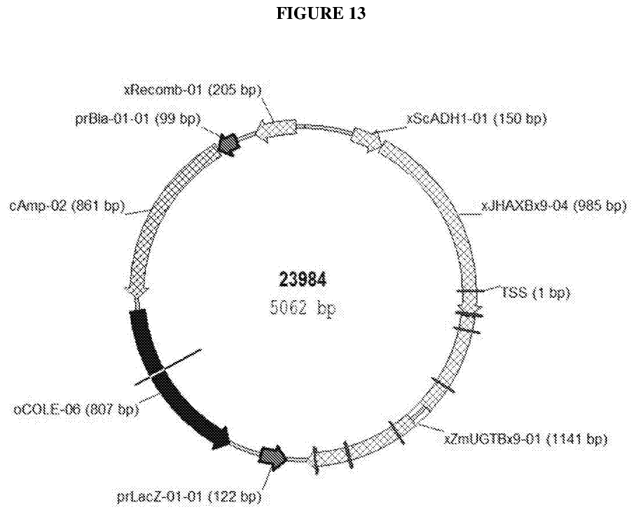

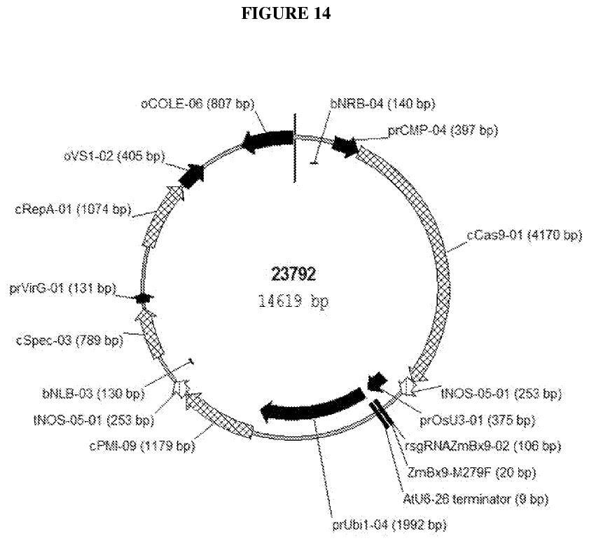

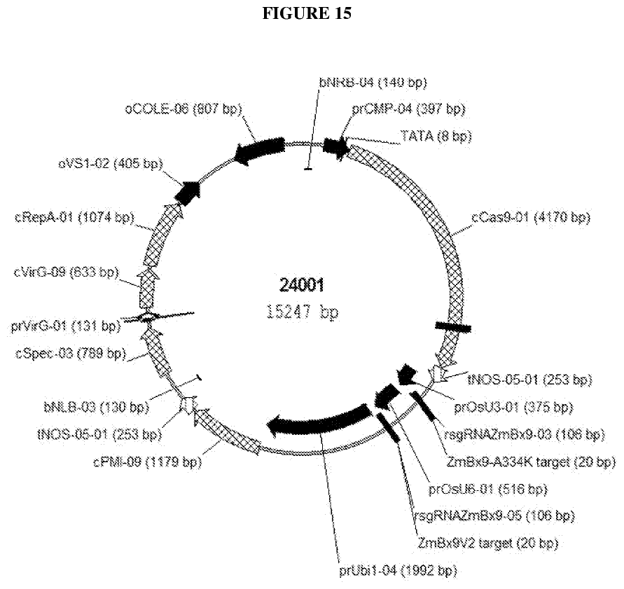

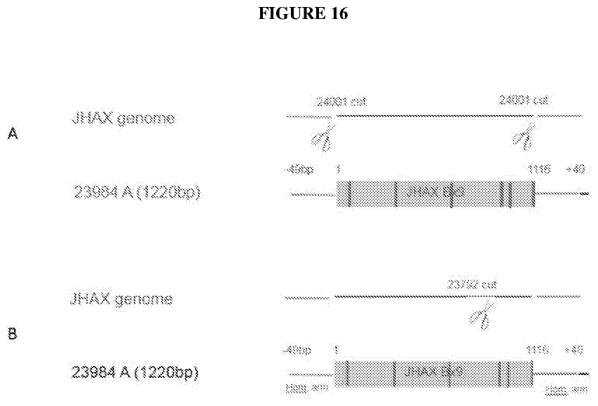

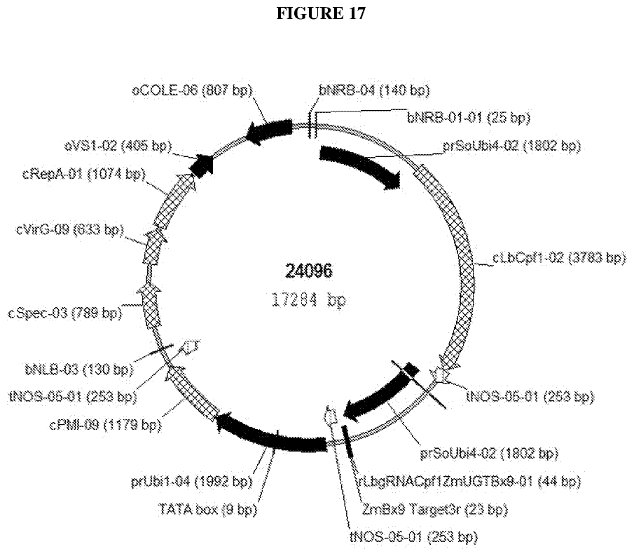

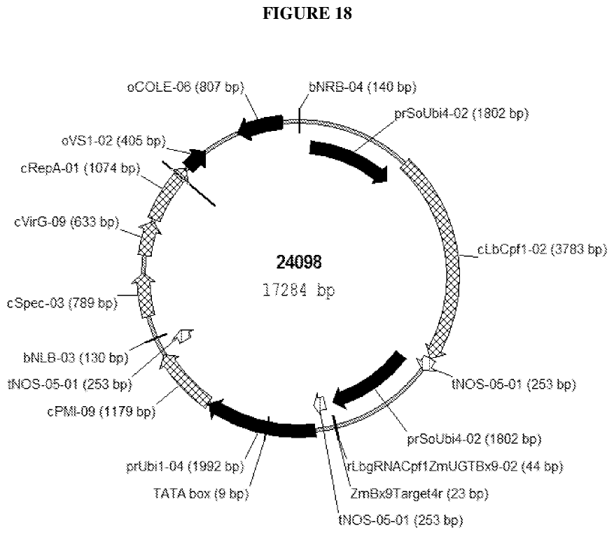

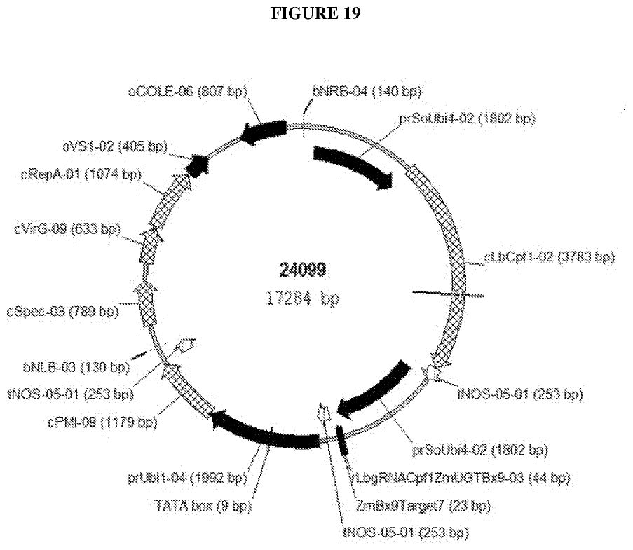

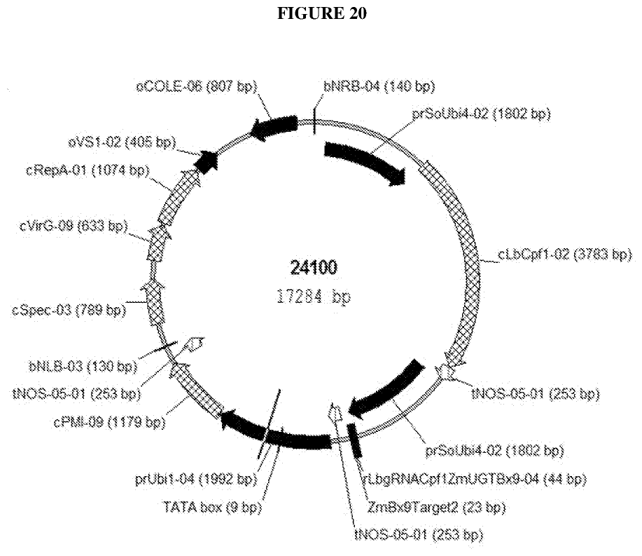

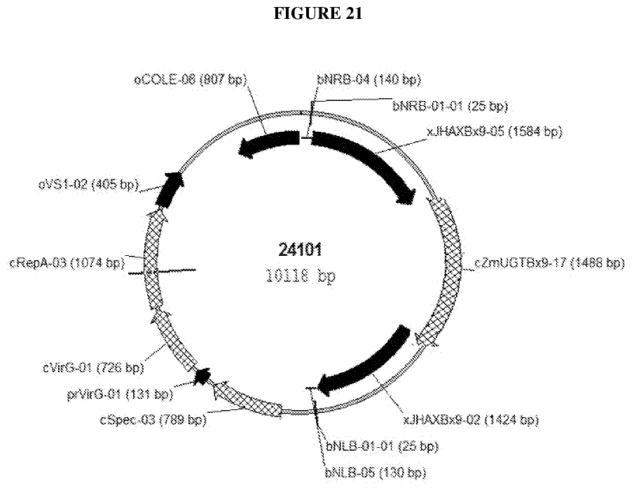

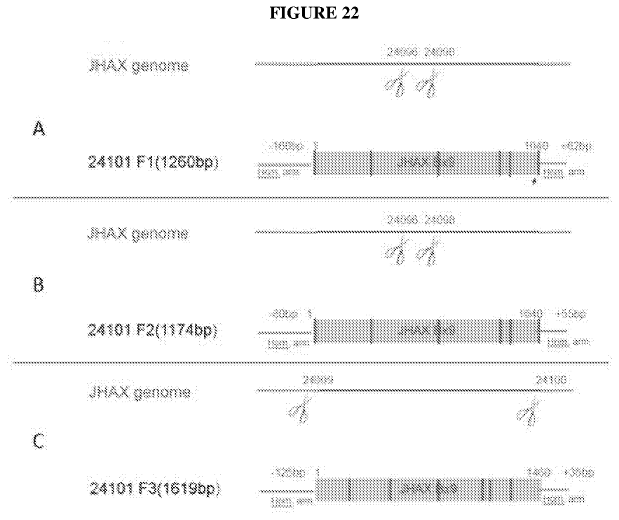

Donor DNA fragment 24001F1 (1.3 Kb) was amplified from template 24001 with forward primer SD61 (5′-GGCAATTGGCAAGTGGACAC-3′) (SEQ ID NO. 189) and reverse primer SD62 (5′-ACCGTTGTGGGTGAGGAAGC-3′) (SEQ ID NO. 190). 24101F1 was designed to include ˜1Kb bp DNA sequence containing 15 bp change from wild type genomic sequence in the middle (cZmUGTBx9-17 SEQ ID No. 179), flanked by 160 bp and 62 bp arms homologous to genomic target locus (xJHAXBx9 SEQ ID No. 180 and cZmUGTBx9 SEQ ID No. 181). Donor 24101F1 were paired with CRISPR vector 24096 and 24098 to achieve gene replacement (FIG. 13A).

Donor DNA fragment 24001F2 (1.2 Kb) was amplified from template 24001 with forward primer SD65 (5′-GCTCACGCTCGGCAGCCATG-3′) (SEQ ID NO. 191) and reverse primer SD66 (5′-TGGGTGAGGAAGCCGCCGAC-3′) (SEQ ID NO. 192). 24101F2 was designed to include ˜1Kb bp DNA sequence containing 15 bp change from wild type genomic sequence in the middle (cZmUGTBx9-17 SEQ ID No. 179), flanked by 80 bp and 55 bp arms homologous to genomic target locus (xJHAXBx9 SEQ ID No. 182 and cZmUGTBx9 SEQ ID No. 183). Donor 24101F2 were paired with CRISPR vector 24096 and 24098 to achieve gene replacement (FIG. 13B).

Donor DNA fragment 24001F3 (1.6 Kb) was amplified from template 24001 with forward primer SD68 (5′-gaatggaccacccgaccgtg-3′) (SEQ ID NO. 193) and reverse primer SD69 (5′-gaatggaccacccgaccgtg-3′) (SEQ ID NO. 194). 24101F3 was designed to include ˜1.5Kb bp DNA sequence containing 19 bp change from wild type genomic sequence in the middle (cZmUGTBx9-17 SEQ ID No. 184), flanked by 125 bp and 35 bp arms homologous to genomic target locus (xJHAXBx9 SEQ ID No. 185 and cZmUGTBx9 SEQ ID No. 186). Donor 24101F3 were paired with CRISPR vector 24099 and 24100 to achieve gene replacement (FIG. 13C).

### Generation of Targeted Gene Replacement Mutant Using Biolistic Bombardment

To generate potential mutants carrying desired sequence replacing wild type bx9 gene, elite maize transformation variety NP2222 was chosen for all experiments as described (US Patent No. 9,133,474 and WO16106121, incorporated by reference herein). Three different CRISPR vector and donor combinations are tested using vector 24096, 24098, 24099, 24100 and donor 24101. Briefly, the same transformation protocol as example 1 was used to co-deliver CRISPR vector and donor DNA to maize were co-delivered to maize immature embryos through biolistic transformation. Plant samples were collected from rooted plants for Taqman assays to detect mutations in the target site and overlapping junction PCRs were performed to identify potential plants containing the targeted gene replacement. Identified putative targeted gene replacement lines will be further characterized by PacBio sequencing.

Table 22 is a summary for gene replacement generation and molecular characterization using Cpf1. There different combinations of Cpf1 CRISPR vectors and donor vector were designed for this study. Donor 24101F1 was designed to have 160 bp and 62 bp homology arms, while 24101F2 and F3 have 80 bp/55 bp, and 125 bp/35 bp homology arms, respectively.

Transformation efficiency for Cpf1 ranged from 4.22%-6.08%, which is comparable to 2.9%-8.5% for Cas9 system, indicating Cpf1 is not toxic to maize tissue culture, which is critical for trait product development in plant biotechnology. High throughput Taqman detected 278 plants with sequence change at the cleavage site when using donor 24001F1 and CRISPR vector 24096 and 24098 for transformation which is the majority of shoots produced from this study, demonstrating efficient cleavage efficiency with Cpf1 system.

Junction PCR data showed that 43 out of these 278 plants (15.46%) achieved gene replacement at least one end of the target gene with 24101F1, while 16.8% and 16.10% of tested plants achieved gene replacement for at least one end of the target gene when using 24101F2 and 24101F3 respectively, indicating the length of homology arms is not critical once it is above a minimum length, which could be as small as 35 bp in this case.

Comparing to junction PCR data generated from example 1 and 2 using Cas9 system, a range of 2.31%-3.38% of tested plants achieved both end gene replacement with Cpf1 system, while less than 1.72% of tested plants achieved both end gene replacement with Cas9 system, except for one single gRNA vector design 23792. This data suggested that Cpf1 might work more efficiently for large gene replacement than Cas9 system. This is probably due to a staggered DSB with a 4 or 5-nt overhang was introduced by Cpf1 at the target site which is favored by homologous recombination, while Cas9 nuclease introduced blunt end double stranded break. Another advantage of applying Cpf1 nuclease for targeted genome editing is the shorter (˜42 nt) crRNA, which is significantly easier and cheaper to synthesize than the ˜100 nt guide RNA in Cas9 based system.

TABLE 22
Comparison of targeted large gene replacement efficiency with Cpf1 and Cas9 system.

| | | Donor | Size L & | Immature | | | | either | % of | Both | % of |
|---|---|---|---|---|---|---|---|---|---|---|---|
| | | size | R Arms | embryo | PMI + | | Events | 5′ or 3′ | either | 5′&3′ | both |
| VC | Donor | (kB) | (bp) | targets | plants | TF % | in GH | PCR+ | PCR+ | PCR+ | PCR+ |
| 23935 | 23939 A | 3.1 | 52, 1428 | 4404 | 299 | 6.80% | 232 | 19 | 8.20% | 4 | 1.72% |
| 23935 | 23939 B | 1.9 | 121, 111 | 1427 | 121 | 8.50% | 135 | 12 | 8.90% | 2 | 1.48% |
| 23792 | 23984 A | 1.2 | 49, 40 | 6666 | 334 | 5.01% | 262 | 20 | 7.60% | 9 | 3.44% |
| 24001 | 23984 A | 1.2 | 49, 40 | 8459 | 247 | 2.92% | 297 | 35 | 11.78% | 3 | 1.01% |
| 24096, | 24101 F1 | 1.3 | 160, 62 | 9294 | 392 | 4.22% | 278 | 43 | 15.46% | 9 | 3.24% |
| 24098 | | | | | | | | | | | |
| 24096, | 24101 F2 | 1.2 | 80, 55 | 6444 | 392 | 6.08% | 303 | 59 | 19.47% | 7 | 2.31% |
| 24098 | | | | | | | | | | | |
| 24099, | 24101 F3 | 1.6 | 125, 35 | 6852 | 334 | 4.87% | 267 | 43 | 16.10% | 7 | 3.38% |
| 24100 | | | | | | | | | | | |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

### BIBLIOGRAPHY

Allison et al., (1986) Virology 154:9-20
Bevan et al., Nature 304:184-187 (1983)
Bevan, Nucl. Acids Res. (1984)
Blochinger & Diggelmann, Mol. Cell Biol. 4: 2929-2931
Bourouis et al., EMBO J. 2(7): 1099-1104 (1983)
Bustos, et al., Plant Cell, 1:839-854 (1989)
Callis et al., Genes Develop. 1:1183-1200 (1987)
Castle et al. (2004) Science, 304:1151-1154
Castle et al. (2004) Science, 304:1151-1154
Chen et al. (2003) Plant J. 36:731-40
Christensen et al. (1989) Plant Mol. Biol. 12:619-632
Christensen et al. (1992) Plant Mol. Biol. 18:675-689
Christou et al. Biotechnology 9: 957-962 (1991)).
D'Ordine et al (2009) J. Mol. Biol., 392, 481-497
Datta et al. Biotechnology 8:736-740 (1990)
Davis et al (1991) Plant Sci., 74, 73-80))
Dayhoff et al. (1978) Atlas of Protein Sequence and Structure (Natl. Biomed. Res. Found., Washington, D.C.),
DeBlock et al (1987) EMBO J., 6, 2513-2518; US 5276268)
Della-Cioppa et al., Plant Physiology 84:965-968 (1987).
Dong et al., 1996, Molecular Breeding 2:267-276
Dutartre et al (2012) BMC Evol. Biol. 12, 64; Dick et al (2012) Plant Cell 24, 915-928;
E. Patrick Fuerst and Michael A. Norman, Weed Science (1991), Vol. 39, No. 3 pp. 458-464).
Edmunds and Morris “Hydroxyphenylpyruvate dioxygenase (HPPD) Inhibitors: Triketones.” In Modern Crop Protection Compounds. 2^{nd} Edition. Eds. Krämer, Schirmer, Jeschke and Witschel. Weinheim, Germany: Wiley-VCH, 2012. Ch. 4.3, pp. 235-262)
Elroy-Stein, O., Fuerst, T. R., and Moss, B. PNAS USA 86:6126-6130 (1989)
Frear et al. (1985) Pest Biochem. Physiol., 23, 56-65).
Frey et al. (2009) Phytochemistry 70, 1645-1651;
Fromm et al. (Biotechnology 8: 833-839 (1990)
Funk et al (2006) PNAS, 103, 13010-13015;
Gallie et al. Nucl. Acids Res. 15: 8693-8711 (1987);
Gallie, D. R. et al., Molecular Biology of RNA, pages 237-256 (1989)
Geiser et al. (1986) Gene 48:109), lectins
Gordon-Kamm et al. (Plant Cell 2: 603-618 (1990)
Green, et al., EMBO J. 7, 4035-4044 (1988)
Guo et al. (2003) Plant J. 34:383-92
Joung and Sander, Nat Rev Mol Cell Biol. (2013) 49-55
Hawkes “Hydroxyphenylpyruvate Dioxygenase (HPPD)—The Herbicide Target.” In Modern Crop Protection Compounds. 2^{nd} Edition. Eds. Krämer, Schirmer, Jeschke and Witschel Eds., Germany: Wiley-VCH, 2012. Ch. 4.2, pp. 225-235;
Hiei et al., 1994, Plant Journal 6:271-282
Hiei et al., 1997, Plant Molecular Biology, 35:205-218).
Hofgen & Willmitzer, Nucl. Acids Res. 16: 9877 (1988)
Hugie et al (2008) Weed Science, 56, 265-270
Innis and Gelfand, eds. (1999) PCR Methods Manual (Academic Press, New York).
Innis et al., eds. (1990) PCR Protocols: A Guide to Methods and Applications (Academic Press, New York); Innis and Gelfand, eds. (1995) PCR Strategies (Academic Press, New York)
Jobling, S. A., and Gehrke, L., Nature 325:622-625 (1987)
Jones et al. (1994) Science 266:789;
Jordano, et al., Plant Cell, 1:855-866 (1989)
Karlin and Altschul (1990) Proc. Natl. Acad. Sci. USA 872264, modified as in
Karlin and Altschul (1993) Proc. Natl. Acad. Sci. USA 90:5873-5877
Klein et al., Nature 327: 70-73 (1987).
Koziel et al. (Biotechnology 11: 194-200 (1993)
Kunkel (1985) Proc. Natl. Acad. Sci. USA 82:488-492;
Kunkel et al. (1987) Methods in Enzymol. 154:367-382;
Lairson et al (2008) Annu. Rev. Biochem., 77, 521-555).
Lamoureux et al (1991) in Herbicide Resistance in Weeds and Crops (J. C. Caseley, G. W. Cussans, R. K. Atkin ed. pp 227-262, Butterworth Heinemann)
Last et al. (1991) Theor. Appl. Genet. 81:581-588
Lommel, S. A. et al., Virology 81:382-385 (1991).
Loutre et al (2003) The Plant Journal, 34, 485-493).
Macejak, D. G., and Samow, P., Nature 353: 90-94 (1991)
Makowska et al (2015) Acta. Physiol. Plant (2015) 37, 176).
Martin et al. (1993) Science 262:1432;
Mayo O., The Theory of Plant Breeding, Second Edition, Clarendon Press, Oxford (1987)
McBride, K. E. et al. (1994) PNAS 91, 7301-7305
McElroy et al. (1990) Plant Cell 2:163-171);
Meier, et al., Plant Cell, 3, 309-316 (1991)
Messing & Vierra Gene 19: 259-268 (1982)
Mets and Thiel (1989) in Target Sites of Herbicide Action (CRC press Boger and Sandmann ed.), pp 1-24)
Mettler, I. J. (1987) Plant Mol Biol Reporter 5, 346349
Mindrinos et al. (1994) Cell 78:1089
Murashige & Skoog, Physiologia Plantarum 15: 473-497 (1962)
Myers and Miller (1988) CABIOS 4:11-17;
Needleman and Wunsch (1970) J. Mol. Biol. 48:443-453; and
Negrotto et al., Plant Cell Reports 19: 798-803 (2000)
Odell et al. (1985) Nature 313:810-812
Paszkowski et al., EMBO J. 3: 2717-2722 (1984),
Potrykus et al., Mol. Gen. Genet. 199: 169-177 (1985) Reich et al., Biotechnology 4: 1001-1004 (1986)
Reed et al., In Vitro Cell. Dev. Biol.-Plant 37:127-132
Rizwan et al (2015) Adv. life sci., vol. 3, pp. 01-08
Sambrook et al. (1989) Molecular Cloning: A Laboratory Manual (2d ed., Cold Spring Harbor Laboratory Press, Plainview, N.Y.).
Schocher et al. Biotechnology 4: 1093-1096 (1986)
Schubert et al. (1988) J. Bacteriol. 170:5837-5847
Shimamoto et al. Nature 338: 274-277 (1989)
Singh, D. P., Breeding for Resistance to Diseases and Insect Pests, Springer-Verlag, NY (1986)
Skuzeski et al. Plant Molec. Biol. 15: 65-79 (1990)).
Smith et al. (1981) Adv. Appl. Math. 2:482;
Spencer et al. Theor. Appl. Genet 79: 625-631 (1990)
Stalker et al (1988) Science, 242(4877):419-23
Svab, Z. and Maliga, P. (1993) PNAS 90, 913-917
Svab, Z., Hajdukiewicz, P. and Maliga, P. (1990) PNAS 87, 8526-8530
Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York.
Uknes et al. Plant Cell 5: 159-169 (1993)
Van Damme et al. (1994) Plant Mol. Biol. 24:825,
Vasil et al. (Biotechnology 10: 667-674 (1992)
Vasil et al. (Biotechnology 11:1553-1558 (1993)
Velten et al. (1984) EMBO J. 3:2723-2730
Walker and Gaastra, eds. (1983) Techniques in Molecular Biology (MacMillan Publishing Company, New York)
Walsh et al (2012) Weed Technol. 26, 341-347;
Weeks et al. (Plant Physiol. 102:1077-1084 (1993)
Welsh J. R., Fundamentals of Plant Genetics and Breeding, John Wiley & Sons, NY (1981) White et al., Nucl. Acids Res 18: 1062 (1990)
Wricke and Weber, Quantitative Genetics and Selection Plant Breeding, Walter de Gruyter and Co., Berlin (1986).
Wood D. R. (Ed.) Crop Breeding, American Society of Agronomy Madison, Wis. (1983)
Zhang et al. Plant Cell Rep 7: 379-384 (1988)
Zhang, et al., Plant Physiology 110: 1069-1079 (1996).

What is claimed is:

1. A recombinant, double-stranded DNA molecule comprising a promoter that drives expression in a plant or plant cell and a polynucleotide that encodes a bx-type glucosyl transferase polypeptide, wherein the promoter is heterologous with respect to the polynucleotide sequence and adapted to cause sufficient expression of the encoded bx-type glucosyl transferase to enhance the herbicide tolerance of a plant cell transformed with the DNA molecule.

2. The DNA molecule of claim 1, wherein the bx-type glucosyl transferase polypeptide comprises at least one mutation at a position corresponding to one of the following amino acid positions of SEQ ID NO: 1:
i. Position 19—mutation to M
ii. Position 21—mutation to Y
iii. Position 22—mutation to any, preferably H,I,P,C or M
iv. Position 78—mutation to any, preferably F or Y
v. Position 79—mutation to any, preferably G,M,E,H,L,F,S,N or Q
vi. Position 86—mutation to any, preferably D
vii. Position 117—mutation to any, preferably T,C,I,V or G
viii. Position 135—mutation to any, preferably S,T,C,H,A,I,L or V
ix. Position 138—mutation to any, preferably S
x. Position 143—mutation to any, preferably Y,F or W
xi. Position 153—mutation to any, preferably T,Q,K,R,V, L, H or F
xii. Position 194—mutation to any, preferably V,I,T,C,N,A,D,G or Q
xiii. Position 220—mutation to any, preferably P,F,R,W,Y,H,K,L,M,E,I,S,N,G or C
xiv. Position 279—mutation to any, preferably I,V,W or F
xv. Position 281—mutation to any, preferably Q,K,R,L,V,M,C,T or S
xvi. Position 334—mutation to any, preferably R or K
xvii. Position 363—mutation to any, preferably S,M,Q,W,T,F,A,V or L
xviii. Position 370—mutation to any, preferably G,S,T,A,F,Y,N,I,A
xix. Position 372—mutation to any, preferably E or Q
xx. Position 376—mutation to any, preferably L
xxi. Position 432—mutation to any, preferably L,V,H,Q,P,T,F,Y,D,E,R,K,N
xxii. Position 437—mutation to a short peptide consisting of or comprising a sequence selected from the group of GIGVD (SEQ ID NO: 102), GIGVDV (SEQ ID NO: 103), GIGVDVD (SEQ ID NO: 104), or GIGVDVDE (SEQ ID NO: 105) or any conservative variant of these sequences.

3. The DNA molecule of claim 1, wherein the bx-type glucosyl transferase polypeptide comprises at least one of the following amino acid motifs:
i. PFPX(Q,L)GH (SEQ ID NO: 61), wherein X=Y
ii. PFPXQGH (SEQ ID NO: 62), wherein X=Y
iii. PFPFXGH (SEQ ID NO: 64), wherein X=any but preferably H,I,P,C,M
iv. ASEDXA (SEQ ID NO: 66), wherein X=any but preferably F,Y
v. ASEDIX (SEQ ID NO: 68), wherein X=any but preferably G,M,E,H,L,F,S,N,Q
vi. (L,M)X(A,D)(S,A)(S,C,A)(D,E)A (SEQ ID NO: 70), wherein X=any but preferably D
vii. LXA(S,A)C(D,E)A (SEQ ID NO: 71), wherein X=any but preferably D
viii. CV(F,L,I)TDVXW (SEQ ID NO: 73), wherein X=any but preferably T,C,I,V,G
ix. PALG(M,V,I)XTASAA (SEQ ID NO:75), wherein X=any but preferably S,T,C,H,A,I,L,V
x. PALG(M,V,I)MTXSAA (SEQ ID NO:77), wherein X=any but preferably S
xi. AY(R,Q)TLXDK(G,A) (SEQ ID NO: 79), wherein X=any but preferably T,Q,K,R,V,L,F,H
xii. E(E,D)FAXLL (SEQ ID NO: 81), wherein X=any but preferably T,C,N,A,D,G,Q,V,I
xiii. IE(T,A)(D,G,A)XL(A,G,E)(Q,R,E)I (SEQ ID NO: 83), wherein X=any but preferably P,F,R,W,Y,H,K,L,M,E,I,S,N,G,C
xiv. IE(T,A)(D,G)XL(A,G)EI (SEQ ID NO: 84), wherein X=any but preferably P,F,R,W,Y,H,K,L,M,E,I,S,N,G,C
xv. VLYVSFGSXAA (SEQ ID NO: 86), wherein X=any but preferably V,W,F,I
xvi. VLYVSFGSMAX (SEQ ID NO: 88), wherein X=any but preferably Q,K,R,L,V,M,C,T,S
xvii. (V,I)VXWAPQEEVL (SEQ ID NO: 90), wherein X=any but preferably R,K
xviii. TVEAX(S,A)EGV (SEQ ID NO: 92), wherein X=any but preferably S,M,Q,W,T,F,A,V,L
xix. EGVPMXC (SEQ ID NO: 94), wherein X=any but preferably G,S,T,A,F,Y,N,I,A
xx. C(C,H)P(R,L)HXDQ (SEQ ID NO: 96), wherein X=any but preferably L
xxi. KIAX(A,D)KG (SEQ ID NO: 98), wherein X=any but preferably L,V,H,Q,P,T,F,Y,D,E,R,K,N
xxii. (R,K,G)(A,M,I,V,S)(E,K,M,L,I,R,G,S,N,H)(E,N,G,D,A,H,V,K,S,Q,I)(L,F,M)(K,G,R,Q, E,M)(S,D,E,Q,G,K,L,N,H,I,M)(R,A,K,V,E,M,I,Q,S)(A,V,S,M)(A,D,E,G,T,S,V,K,E,L,I, Y,R,N)(K,R,L,V,F,Q,S,D,E,A)(G,C,S,A,T)(I,T,A,L,V,F,M,S) (SEQ ID NO: 99), adjacently linked to a short peptide that either consists of or comprises at its N terminus a sequence selected from the group of GIGVD (SEQ ID NO: 102), GIGVDV (SEQ ID NO: 103), GIGVDVD (SEQ ID NO: 104), or GIGVDVDE (SEQ ID NO: 105)
xxiii. R(A,M)(K,M,L,I,R,G,S,N,H)(E,N,G,D,A,H,I)(L,F,M)(K,G,R,Q)(S,D,E,Q,G,K,L,N,H,I, M)(R,A,K,V,E,M,I,S)(A,V,S,M)(A,D,E,G,T,S,V,K,E,L,I)(K,R,Q,S,D,E,A)(G,C,S,A,T)(I, T,A,L,V,M,S) (SEQ ID NO: 100) adjacently linked to a short peptide consisting of or comprising at its N terminus a sequence selected from the group of GIGVD (SEQ ID NO: 102), GIGVDV (SEQ ID NO: 103), GIGVDVD (SEQ ID NO: 104), or GIGVDVDE (SEQ ID NO: 105) or any conservative variant of these sequences.
xxiv. R(A,M)(K,M,L,I,G,N,H)(E,N,G,D,A,H)(L,M)(K,G,R,Q)(S,D,E,Q,G,K,L,N,H,I,M)(R,A, K,V,E,M,I)(A,V)(A,D,E,G,S,V,L)(K,R,Q,D,E)(G,C,S,A)(I,T,A,V) (SEQ ID NO: 101) adjacently linked to a short peptide consisting of or comprising at its N terminus a sequence selected from the group of GIGVD (SEQ ID NO: 102), GIGVDV (SEQ ID NO: 103), GIGVDVD (SEQ ID NO: 104), or GIGVDVDE (SEQ ID NO: 105) or any conservative variant of these sequences.

4. The DNA molecule of claim 1, wherein the polynucleotide sequence encodes a bx-type UDP glucosyl transferase selected from the group consisting of SEQ ID NOs: 1-54.

5. The DNA molecule of claims 1-3 wherein the polynucleotide sequence is optimized for expression in a plant or plant cell.

6. The DNA molecule of claims 1-3, further comprising an operably linked isolated polynucleotide sequence encoding a polypeptide that confers a desirable trait.

7. The DNA molecule of claim 6, wherein the desirable trait is resistance or tolerance to an herbicide.

8. The DNA molecule of claim 6, wherein the desirable trait is resistance or tolerance to one or more insects.

9. The DNA molecule of claim 6, wherein the desirable trait is resistance or tolerance to an abiotic stress.

10. The DNA molecule of claim 7, wherein said desirable trait is resistance or tolerance to an HPPD inhibitor, glyphosate, glufosinate, an auxin herbicide or a PSII inhibitor herbicide.

11. The DNA molecule of claim 6, wherein said polypeptide that confers a desirable trait is a cytochrome P450 or variant thereof.

12. The DNA molecule of claim 6, wherein said polypeptide that confers a desirable trait is an EPSPS (5-enol-pyrovyl-shikimate-3-phosphate-synthase).

13. The DNA molecule of claim 6, wherein said polypeptide that confers a desirable trait is a phosphinothricin acetyl transferase (PAT).

14. A vector comprising the DNA molecule of any one of claims 1-3.

15. A method for conferring resistance or tolerance to an herbicide in a plant, the method comprising introducing the DNA molecule of any one of claims 1-3 into the plant.

16. The method of claim 15, wherein the herbicide is an amine, alcohol or aminal herbicide selected from the group consisting of structures III, IV, V, VI, VII, VIII, IX, X, XI, XII, XIII, XIV, XV, XVI, XVII, XVIII, XIX, XX, XXI, XXII, XXIII, XXIV, XXV, XXVI and metribuzin.

17. The method of claim 16 wherein the herbicide is structure V, VI or metribuzin.

18. A plant cell comprising the DNA molecule of any one of claims 1-3.

19. The plant cell of claim 18, wherein the plant cell is a from a plant selected from the group comprising rice, barley, potato, sweet potato, canola, sunflower, rye, oats, wheat, corn, soybean, sugar beet, tobacco, Miscanthus grass, Switch grass, safflower, trees, cotton, cassava, tomato, sorghum, alfalfa, sugar beet, and sugarcane.

20. The plant cell of claim 19, wherein the plant cell is a soybean or corn plant cell.

21. A plant, plant part, or seed comprising the plant cell of claim 18.

22. A method of controlling weed growth in a crop growing environment comprising a plant or seed of claim 21, the method comprising applying to the crop growing environment an amount of an amine, alcohol or aminal herbicide effective to control weed growth.

23. The method of claim 22, wherein the herbicide is structure V, VI or metribuzin.

24. The method of claim 22, wherein the herbicide is applied over the top of the crop growing environment.

25. A method of producing food, feed, or an industrial product comprising:
a. obtaining a plant, plant part or seed of claim 21; and
b. preparing the food, feed or industrial product from the plant, plant part or seed.

26. The method of claim 25, wherein the food or feed is oil, meal, grain, starch, flour or protein.

27. The method of claim 25, wherein the industrial product is biofuel, fiber, industrial chemicals, a pharmaceutical or nutraceutical.

28. A method for introducing a herbicide tolerance trait into a plant, comprising:
i. selecting a plant comprising a nucleic acid sequence in its genome that encodes a bx-type UDP glucosyl transferase polypeptide; and
ii. introducing a modification to the nucleic acid sequence such that the encoded polypeptide comprises at least one of the mutation at a position corresponding to one of the following amino acid positions of SEQ ID NO: 1:
iii. Position 19—mutation to M
iv. Position 21—mutation to Y
v. Position 22—mutation to any, preferably H,I,P,C or M
vi. Position 78—mutation to any, preferably F or Y
vii. Position 79—mutation to any, preferably G,M,E,H,L,F,S,N or Q
viii. Position 86—mutation to any, preferably D
ix. Position 117—mutation to any, preferably T,C,I,V or G
x. Position 135—mutation to any, preferably S,T,C,H,A,I,L or V
xi. Position 138—mutation to any, preferably S
xii. Position 143—mutation to any, preferably Y,F or W
xiii. Position 153—mutation to any, preferably T,Q,K,R,V, L, H or F
xiv. Position 194—mutation to any, preferably V,I,T,C,N,A,D,G or Q
xv. Position 220—mutation to any, preferably P,F,R,W,Y,H,K,L,M,E,I,S,N,G or C
xvi. Position 279—mutation to any, preferably I,V,W or F
xvii. Position 281—mutation to any, preferably Q,K,R,L,V,M,C,T or S
xviii. Position 334—mutation to any, preferably R or K
xix. Position 363—mutation to any, preferably S,M,Q,W,T,F,A,V or L
xx. Position 370—mutation to any, preferably G,S,T,A,F,Y,N,I,A
xxi. Position 372—mutation to any, preferably E or Q
xxii. Position 376—mutation to any, preferably L
xxiii. Position 432—mutation to any, preferably L,V,H,Q,P,T,F,Y,D,E,R,K,N
xxiv. Position 437—mutation to a short peptide consisting of or comprising a sequence selected from the group of GIGVD (SEQ ID NO: 102), GIGVDV (SEQ ID NO: 103), GIGVDVD (SEQ ID NO: 104), or GIGVDVDE (SEQ ID NO: 105) or any conservative variant of these sequences.
wherein a site-directed nuclease (SDN) introduces the modification to the nucleic acid sequence.

29. The method of claim 28, wherein the SDN is selected from the group comprising: meganucleases, zinc finger nucleases (ZFN), transcription activator-like effector nucleases (TALEN) or Clustered Regularly Interspaced Short Palindromic Repeats system (CRISPR)

30. A plant produced by the method of claim 28.

31. The DNA molecule of claim 2, comprising from at least two to at least six mutations.

32. The DNA molecule of claim 3, comprising from at least two to at least six amino acid motifs.

33. The DNA molecule of claim 4, wherein the polynucleotide sequence encodes a bx-type UDP glucosyl transferase having the sequence set forth in SEQ ID NO: 16.

34. The DNA molecule of claim 4, wherein the polynucleotide sequence encodes a bx-type UDP glucosyl transferase having the sequence set forth in SEQ ID NO: 20.

35. A polypeptide having 60% identity to SEQ ID NO: 1 and having a combination of amino acids at the positions corresponding to the positions of SEQ ID NO:1, wherein the combination is selected from the group comprising:
I. Combination A
i. Position 21—F or Y
ii. Position 117—V
iii. Position 194—V
iv. Position 279—F
v. Position 281—K
vi. Position 334—K
II. Combination B
i. Position 21—F or Y
ii. Position 117—V
iii. Position 194—V
iv. Position 279—F
v. Position 334—K
III. Combination C
i. Position 21—F or Y
ii. Position 117—V
iii. Position 220—P
iv. Position 279—F
v. Position 334—K
IV. Combination D
i. Position 117—V
ii. Position 279—F
iii. Position 334—K
V. Combination E
i. Position 117—V
ii. Position 279—F
iii. Position 334—R
VI. Combination F
i. Position 279—F
ii. Position 432—P
VII. Combination G
i. Position 117—G
ii. Position 143—F
iii. Position 279—W
iv. Position 432—F

36. The polypeptide of claim 35, having at least 70% sequence identity to SEQ ID NO: 1.

37. The polypeptide of claim 35, having at least 80% sequence identity to SEQ ID NO: 1.

38. The polypeptide of claim 35, having at least 90% sequence identity to SEQ ID NO: 1.

39. The polypeptide of claim 35, having at least 95% sequence identity to SEQ ID NO: 1.

40. A polypeptide having an amino acid sequence selected from the group consisting of SEQ ID Nos 16-54.

41. A compound selected from

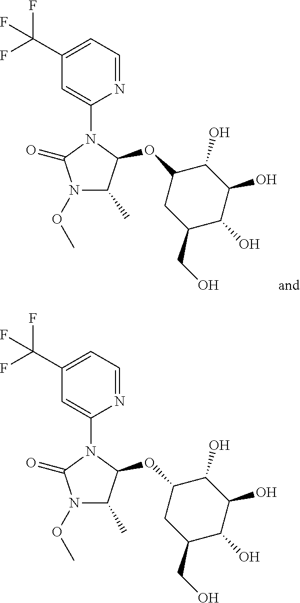

42. A compound selected from

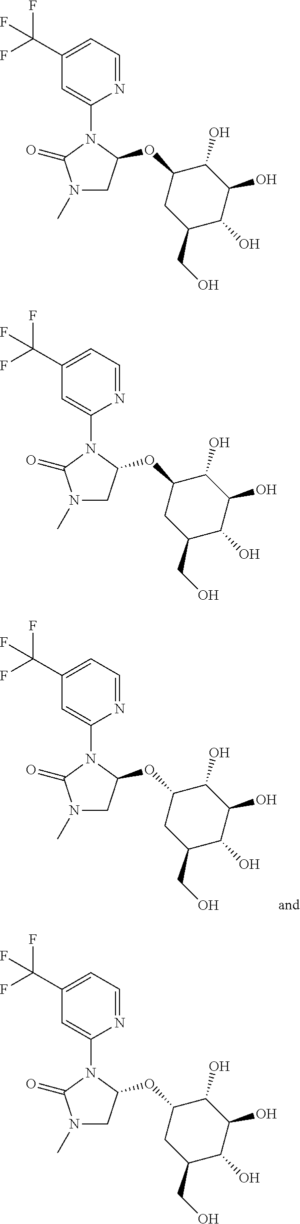 [SMILES: CN1C[C@H](O[C@@H]2C[C@H](CO)[C@@H](O)[C@H](O)[C@H]2O)N(c2cc(C(F)(F)F)ccn2)C1=O ; CN1C[C@@H](O[C@@H]2C[C@H](CO)[C@@H](O)[C@H](O)[C@H]2O)N(c2cc(C(F)(F)F)ccn2)C1=O ; CN1C[C@H](O[C@H]2C[C@H](CO)[C@@H](O)[C@H](O)[C@H]2O)N(c2cc(C(F)(F)F)ccn2)C1=O ; CN1C[C@@H](O[C@H]2C[C@H](CO)[C@@H](O)[C@H](O)[C@H]2O)N(c2cc(C(F)(F)F)ccn2)C1=O]

43. A method of a preparing a compound having a structure selected from the group consisting of: 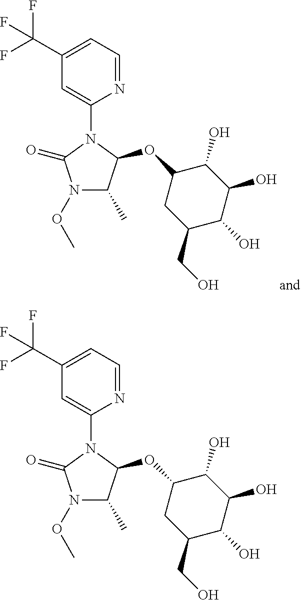
the method comprising steps of providing a bx-type glucosyl transferase polypeptide and contacting said polypeptide with a compound having the structure 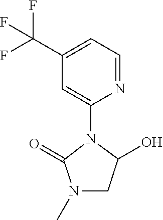 [SMILES: CN1CC(O)N(c2cc(C(F)(F)F)ccn2)C1=O]


SEQUENCE LISTING (186 sequences)

SEQ ID NO: 1 - protein, 462 aa, Zea mays
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSMAAMDPHEFVELAWGLADSKRPFVWVVRPNLIRGFESGALPDGVEDEVRGRGIVVAWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 2 - protein, 459 aa, Zea mays
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 3 - protein, 451 aa, Echinochloa phyllopogon
MAGAAGRRVVFFPFPFQGHFNPLMRLASALHAHGLAITVFHTELRSPDPADYPADYRFVSLPVEVPRELVASEDIAGIVTAMNASCEAPFRDRLAALLAADGVRCVVTDVAWYAAEAVARDLGVPALGVMTASAASFRVYMAYPTLIDKGYLPVREEHKDDPVHELPPYRVKDLLRHDTCRLADFAGLLRRMIAGARHSSGLIINTFAAIEASNLDEIHEHMSIPVFAIAPLNKLATSAGTSLYGETQPDRRCLHWLDTQQPGTVLYVSFGSLAAMDPEQFVELAWGIAESKQPFLWVVRPKLIRGFESGELPEGIEEAVHDRGMIVSWAPQEEVLGHPAVGAFFTHSGWNSTMEAMSEGVPMICHPLHGDQFGNARYVCDVWKVGVELDGGSKLERGKIKAAIEKMVGSKDGEEIRERMKNLKMAAEKGIDEGGSSHADLLKLVDLVKSF

SEQ ID NO: 4 - protein, 456 aa, Triticum aestivum
MAGAGRRRVVFFPFPFLGHFNPVLRLAGALHARGLAVTVFHTEQRVPDPADYPAGYRFVPLPVEVPPELAASEDIARMGMAMNDAAEAPFRDRLAALLAEEAGEDGGVLCVITDVVWYSAQAVARELGVPALGIMTASAAIFRVYMAYQTLIDKAYLPVQDARKDDPVEELPPYLVKDLLRHDTSKLEDFAELLRHTVAGARQSSGLIINTLGAIEAANLERIREDLSVPVFAVAPLHKLAPSAKSSSLGETQADRGCLGWLDTQEPGSVLYVSFGSLAAMDPHEFVELAWGLALSKRPFVWVVRPKLIRGFESGELPDGLGEELRGRGVIVSWAPQEEVLAHPAVGAFFTHSGWNSTVEAIAEGVPMICHPLHGDQYGNARYVADVWRVGVEVDGSHRLERGSIKAAIGRMMESGEGREIGERMKALKMAAEDGIGERGSSHTHLSDLVALIKSF

SEQ ID NO: 5 - protein, 463 aa, Sorghum bicolor
MDAMEPSSGADAGRRVVLFPLPYQGHLNPMLRLAAALHRRGLAIIVLHTDLQPLDPANHPTEYRFESLSADVPAELMASKDIARVVMDLNASFAAPFKDRVAALVADKESGGVDCVITDAVWFSAQAAAQELGVPSLGLFTNSAASFRTFMAYPTLIEKGYLPVQESKKDDPVPELPPFRVKDLERIDTSSLYDFASMVGNVVARARQASGLILNSFDAIEANNVNKIREELSIPVFAVGPLNKLSPSVVKTNLMPGGRECLDWLDTQAPGSVLYVSFGSIAAMDAPDFVELAWGLADSKRPFVWAVRPSLVRSRSLESAKLQLPDGLEEEIHGRGKIVYWAPQEEVLSHPAICAFLTHNGWNSTVESISQGVPMLCRPCFGDQFGTARYVCDFWKVGVEIGVVTQLRRGNIRAAIDKLMDDKQAKEYRDRAKDLKEMAEKCATKDGSSHRALVSLVDFIISF

SEQ ID NO: 6 - protein, 461 aa, Hordeum vulgare
MAPSSTNRGRRRVVLLPLPYQGHINPMLRLAAALHSRGLAVTILHPETRAPDRRKLPADYRLVTIPDNIPPELAASEDIASFVFALNKNCAAPFRDYLAGALREEEEGEDGRLAFVVADVDWFAPLSVARELGVAALALMTTSAARFLVYLAYPSLCQKGYLPVQESNLNTRVEELPPFVFVVRDLDCVKDMAGHLAYADLLAHIVAGVRQSSGLIINTFEDIGGMEIERIRSEITLPVFAVGPLHMMTSSSSVESSLLTEDRSCLDWLDTQRSNSVLYVSFGSLVGIDTDEFLEMAWGLADSQNPFLWVVRPGLVRGSESNTLPGELQEKMGNKGRIVSWAPQQEVLRHPSVAAFLTHCGWNSTTESISEGVPMICRPLFGDQMGTARYVCDVWKVGVRVEVENQLKRGNVQTAITRLMDGKEGEEVRERMRDLRHVVLKCTSEGGTSDVALQRLVDSNV

SEQ ID NO: 7 - protein, 458 aa, Alopecurus pratensis
MAPEGTGDRRRRVLIFPLPYQGHMNPMFHLAGLLHARGLAVTVFHTDSNAPDPSSYPEYDFVAVPDGMPAATQQVLKVMEHVLALNRSCEEPFRERLAALLEAPGARDDVACLVADAHLLTLMDVARQQGVPTLALRTGSAASFLCFVAHPMLCDKGYLPPQESQLDAPVKEMPPYRVRDLMSARSSGGEHDVSCELLSRAVTAVRTSSGLILNTFDALEAADLAAIRRDLAPPVFDIGPLHKVSPAASASSSSLLRQDHGCLEWLNSRAPATVLYVSFGSLASVSSADLVETAWGIALSGQPFLWALRPDLVLGATRAALPDGFEAATAGRGMVVSWAPQEEVLAHAAMGAFWTHNGWNSTLESICEGVPMICRPHFADQMINARYVQEVWKVGFELVGKLERGMIETGVRKLMCQEEGEELRRRASILKDKAGLCIKKGGSSEANIDLLVNRIMSL

SEQ ID NO: 8 - protein, 454 aa, Avena sativa
MASVNGDSRRARRVLVFPLPFQGHINPMMQLADVLHSRGLGVTVLHTRFNALDPALHPEYAFVAVPDGIPADVAASGSIISIILAMNAAMEASGAVHDVLASVLADGPAACLFIDANLLAVQKAAAALGLPTMVLRTGSAACFSCFLAYPMLHEKGYLPPKESQLYTPVDELPPLRVRDLFFSSSNNHEMVRQVLARATETVRNSHGLVINTFEALESAELDRIRRELEVAVVLAAGPLHKLSTRGNGSSLLQQDRTCIEWLDTQAAGSVLYVSIGSLASMDPGELSEVAWGLANSGQPFLWVVRPDLVPGSDGSGLSEGFHRAVEGRAKVIPWAPQQEVLAHSAVGGFWTHNGWNSTLESISEGHPMICRPQFADQMMNTRYVEATWGVGFELEGKLERNKIEEAIRNLMKGSQGELARERARELKKKVISCLESDGSSSLAIDKLIEHMLSL

SEQ ID NO: 9 - protein, 454 aa, Oryza sativa
MAPPCGRVVLFPMPYPGHTIPMFHLAAVLRSRGFSITVLHTELRAPDPAAHPPEYRFVAVADGTPPELVVSEDAAAVLTSLNETCAAPFADRLAALLAEEGGVLCVIADVMWYAPAAAAPELGVPLMLLMTSSASSFRTFMEYPLLLERGFLPVDDAQKDTLVDILPPFRVKDLQRIDTTNLYSFANVLANVVAAARLSSGLILNTFDFIEGDNICRIRDELSIPVFAIGPLNKLIPLVGRSSFLPPDCDCLRWLDTQAPSSVLFVSFGTMATIDAQEFLEVAWGLAGTKLPFLWVVRPSLVRGLRLHSSELPSDLQEEINGRGRIVSWAPQEKVLGHPSVRAFMTHNGWNSTIESISEGVPMICRPCFGDQMGNARYVCAVWRLGVEMEVGSVLQRAKVQTAVEKLVNGEEGQNVKQRMRNLRIEAEKCVSKGGSSDTGLRNLVDSILSFGKC

SEQ ID NO: 10 - protein, 477 aa, Consolida orientalis
MASSSPKTPHIVCVPAPAQGHINPMFKLAKLFHSRGFYITFVHSEFSYQRLLQASALDHLKGLNNFRFETIPDGLPPENKRGVSDVPELCKSMRNTCADPFRSLILKLNSSSDVPPVTCIVADVAMDFTLQVSEELGPPVVLFFTLSGCGVLGYMHYGELLERGYFPLREESFLSNGYLDTEIDWIPAMKGIRLKDLPSFLRTTDPDDIMFNCKIIEVNSAFKAKGVILNTFDDLEQEVLDAIKSKIPQLYTIGPLSMLCDHMLQPDSKLCEASLWEEDTSCLEWLQEKDPKSVLYVNIGSLATMTSQQLGEFAWGLANSMCPFLWVIRPDILDRASGIVSEDYKKEIGGRGLLVSWCQQEKVLKHPSIGGFLTHCGWNSTLESLCEGVPMICWPFFAEQQTNCFYICNKWGIGMEIDFDVKRVEIGMMVKELMKGEKGLEMRNKVEDLMSKAIKATTPGGSSHTNFEMLMEDVAKW

SEQ ID NO: 11 - protein, 454 aa, Secale cereale
MAGAPRRVVFFPFPFLGHFNPVLRLAGALHARGLAVTVFHTEQRVPDPADYPADYRFVSLPVEVPPELVTSEDIARMGMAMNDASEAPFRDRLAALLAKEAEDGGVLCVISDVVWYSAQAVARELGVPALGIMTASAAIFRVYMAYQTLIDKAYLPVQDARKDDPVEELPPYLVKDLLRHDTSRLEDFAELLRHTVAGARQSSGLIINTLGAIEADNLQQIREDLSVPVFAVAPLHKLAPSAKAGSLGDTQADRGCLDWLDTQNPGTVLYVSFGSLAAMDPHEFVELAWGLAQSKRPFVWVVRPKLIRGFESGELPDGLGEELSRRGKIVSWAPQEEVLAHPAVGAFFTHSGWNSTVEAISEGVPMICHPLHGDQYGNARYVSDVWKVGVEVDGTHRLERGSIKAAIERMMDSSEGQEIRERMKGLKMAADDGINERGSSHTHLSDLVALIKSF

SEQ ID NO: 12 - protein, 477 aa, Brachypodium arbuscula
MASPETNTTTNASAGAGHGGGHGRHVLVFPLPYQGHINPMFRLAGILHARGFAVTVFHTQFNAPDPARHPEYRFVPVPVAEDCDKGVVSGPGAGEGIDGVVSHILALNAASESPFLDRLRAVLEEYSRDAVSCLVVDGHLLSMVHVAARLALPSLVLRTGSAACFSCFLAYPSLIAQGYLPLQMEDEVSELPPYRVRDLMRLGKHELTRELLARSVAAVDASAGLILNTFDALEQPELAKLRRDLGGGIPVFDVGPLHMLSPSAGASSSLLRADGTCLAWLDAHAPASVLYVSFGSLACMTARELVETAWGIAGSGVAFLWVVRPGMVAGSEGLATMPEGFEEATRERGKVVEWAPQEDVLRHAAVGGFWTHNGWNSTTESVCEGVPMLCRPHFGDQTGNARYVEHVWKVGFEVVGAGEELERGKVEKAIRRLVVEKDGGEMRARAGELRKKAVECTGKGGSSDLAVDALVKHMMSL

SEQ ID NO: 13 - protein, 463 aa, Eleusine indica
MTTTPTSSGGPQRRRRVLMFPLPFQGHINPMMQLAGVLHARGGLDITFFHAAFNAPDPARRPAGYRFVPVGEGVPSGDLLPSGGDRDFVAALLRINERLASPFRDLLKRELAADDAAACLVVDSNLRGMQLMAEELGLPTLVLRPGAAACLVAYMAFPALCEKGLLPPSTQDQSQLDKLLDEVPPLRLRDMMFSSATSHANMCKCIECLVECSSHSSGVIINTFLDLEDHELQKVTDVLNIPAYAIGPLHKISLGAESSLVEQDWTCLEWLDKQADASVLYVSFGSLASMEENELLETAWGLANSQRPFLWVIRHNLVQSSEQAGLPDAFKEVTHDRGMIVSWAPQQQVLEHRAVGGFWTHNGWNSTVESICEGVPMICRPQFADQMINMRYVQEVWKIGFELEGELERASIERAIQRLFSKEEGREMKHRAMKLRKKAVKCMKEGGSSKTSIDLLVKRIMSF

SEQ ID NO: 14 - protein, 460 aa, Setaria italica
MASPTIRAGRRVVFFPLPYQGHFNPMLRLAGALHARGVAVTVFHTDLRAPDPTYYPSDYRFVPVPVHVPTELVGSEDIARFVMELNVSCAAPFKERLAALLAGEEEEEEAGGVQCVITDVIWYSAQAAARELGVPALGLMTSSAASFRNIMVYPTLIEKCYLPVQEEHKDDPVDVLPPFRVRDLQRIETSSLADFASLLEHTVDGGRQSAGLIINTVEAIEAVDLDKIREDMPIPVFPIGPLNMVSPPVESSLYQLQQDRRCLDWLDTKAPGSVIYVSFGSLAAMDPHEFAELAWGLADSKRPFIWVVRPSLIRGSESGDLPEGFREEIGDRGRIVDWAPQDEVLAHPAVCAFLTHNGWNSTMEAISQGVPMISRPFFGDQYGNAMFVCHVWRVGVEVQVENQLERGKVRDAIEKLMGSKEGKEIGERMMNLKEIAEKGIKESGSSHTAFLNLADLIFSL

SEQ ID NO: 15 - protein, 450 aa, Dicanthelium oligosanthes
MKPSNGPCGCPRVLLFPLPYQGHINPMLRLAAALHARGFAITVVHTETRAPDRRKLPVEYELVTIRDGVPPELAESDDVVPFVLALNRSCAAPFREYLAGARVACVVADVDWFAPLAAARDLGVKALPLMTSSAAKFRVYLAFPILEEKGYLPIRESNLDTEVKELPPLLVRDLHHEKDTTRYHAYADLLSHMVAGVRQSSGLILNTLDAIEGTDIANIYRDISLPVFAIGPLHILSPSVDSSLLLQDRSCLEWLDAQPPGSVIYASLGSLMSIDVHELTEMAWGLAGSKHPFLWVVRQGLVHGCEFSEVSNELQEEIRDRGRIVSWAPQQEVLKHPAMGAFLTHCGWNSTLESILEGVPMICRPLGGDQLCNARYVCEVWKVGIRVVEVENQLTRGDVQVAIERLMGQNEGEEVRERMIDLRDAAAKCTSKGESTDVSLQRLVDFIVST

SEQ ID NO: 16 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVRWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 17 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 18 - protein, 462 aa, Artificial Sequence
  Mutated Zea mays Bx9 protein sequence
MASSRTGAGAGGRVVVFPFPYQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAKMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 19 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAEVLARTVTAARRASGLIFNTFPLIETDTLAEIHKALSVPVFAVAPLNKLVPTATASLHGVVQADRGCLQWLDTQQPGSVLYVSFGSFAKMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 20 - protein, 462 aa, Artificial Sequence
  Mutated Zea mays Bx9 protein sequence
MASSRTGAGAGGRVVVFPFPYQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAEVLARTVTAARRASGLIFNTFPLIETDTLAEIHKALSVPVFAVAPLNKLVPTATASLHGVVQADRGCLQWLDTQQPGSVLYVSFGSFAKMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 21 - protein, 462 aa, Artificial Sequence
  Mutated Zea mays Bx9 protein sequence
MASSRTGAGAGGRVVVFPFPYQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAEVLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 22 - protein, 462 aa, Zea mays
MASSRTGAGAGGRVVVFPFPYQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDPLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 23 - protein, 462 aa, Zea mays
MASSRTGAGAGGRVVVFPFPYQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAECLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 24 - protein, 462 aa, Zea mays
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAECLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 25 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVCWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVRWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 26 - protein, 462 aa, Artificial Sequence
  Mutated Zea mays Bx9 protein sequence
MASSRTGAGAGGRVVVFPFPYQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAEVLARTVTAARRASGLIFNTFPLIETDPLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 27 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSMAAMDPHEFVELAWGLADSKRPFVWVVRPNLIRGFESGALPDGVEDEVRGRGIVVAWAPQEEVLAHPAVGGFLTHNGWNSTVEAWSEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 28 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVVWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVKWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAPAKGIGIGVDVDETASPRTDLTDLVDLIKSF

SEQ ID NO: 29 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVAWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAPAKGIGIGVDVDETASPRTDLTDLVDLIKSF

SEQ ID NO: 30 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASFRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAAMDPHEFVELAWGLADSKRPFVWVVRPNLIRGFESGALPDGVEDEVRGRGIVVAWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAFAKGIGIGVDVDETASPRTDLTDLVDLIKSF

SEQ ID NO: 31 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVVWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 32 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSFAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 33 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAKMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 34 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAWSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 35 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAPDKGIDESAGSDLTNLVHLINSY

SEQ ID NO: 36 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVVWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSFAAMDPHEFVELAWGLADAGRPFVWVVRPNLIRGFESGALPDGVEDRVRGRGVVVKWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 37 - protein, 465 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVVWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSFAAMDPHEFVELAWGLADAGRPFVWVVRPNLIRGFESGALPDGVEDRVRGRGVVVKWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIGIGVDVDESAGSDLTNLVHLINSY

SEQ ID NO: 38 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAISEGVPMICCPRHGDQYGNARYVCHVWKVGTEVAGDQLERGEIKAAIDRLMGGSEEGEGIRKRMNELKIAADKGIDESAGSDLTNLVHLINSY

SEQ ID NO: 39 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAISEGVPMVCHPRHGDQYGNARYVCHVWKVGTEVAGDQLERGEIKAAIDRLMGGSEEGEGIRKRMNELKIAADKGIDESAGSDLTNLVHLINSY

SEQ ID NO: 40 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMVCCPRHGDQYGNARYVCHVWKVGTEVAGDQLERGEIKAAIDRLMGGSEEGEGIRKRMNELKIAADKGIDESAGSDLTNLVHLINSY

SEQ ID NO: 41 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMPSEDIAAIVTALNAACEAPFRDRLSALLSAADGEAGEAGGRVRCVLTDVSWDAVLSAARGLGVPALGVMTASAATFRVYMAYRTLVDKGYLPVREERKEDAVPELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 42 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPTATASLHGVVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 43 - protein, 459 aa, Artificial Sequence
  Mutated Zea mays Bx8 protein sequence
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGVVQADRGCLQWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 44 - protein, 456 aa, Artificial Sequence
  Mutated Wheat Bx8 protein sequence
MAGAGRRRVVFFPFPFLGHFNPVLRLAGALHARGLAVTVFHTEQRVPDPADYPAGYRFVPLPVEVPPELAASEDIARMGMAMNDAAEAPFRDRLAALLAEEAGEDGGVLCVITDVVWYSAQAVARELGVPALGIMTASAAIFRVYMAYQTLIDKAYLPVQDARKDDPVEELPPYLVKDLLRHDTSKLEDFAELLRHTVAGARQSSGLIINTLGAIEAANLERIREDLSVPVFAVAPLHKLAPSAKSSSLGETQADRGCLGWLDTQEPGSVLYVSFGSLAAMDPHEFVELAWGLALSKRPFVWVVRPKLIRGFESGELPDGLGEELRGRGVIVSWAPQEEVLAHPAVGAFFTHSGWNSTVEAIAEGVPMICHPLHGDQYGNARYVADVWRVGVEVDGSHRLERGSIKAAIGRMMESGEGREIGERMKALKMAPEDGIGERGSSHTHLSDLVALIKSF

SEQ ID NO: 45 - protein, 456 aa, Artificial Sequence
  Mutated Wheat Bx8 protein sequence
MAGAGRRRVVFFPFPFLGHFNPVLRLAGALHARGLAVTVFHTEQRVPDPADYPAGYRFVPLPVEVPPELAASEDIARMGMAMNDAAEAPFRDRLAALLAEEAGEDGGVLCVITDVVWYSAQAVARELGVPALGIMTASAAIFRVYMAYQTLIDKAYLPVQDARKDDPVEELPPYLVKDLLRHDTSKLEDFAELLRHTVAGARQSSGLIINTLGAIEAANLERIREDLSVPVFAVAPLHKLAPSAKSSSLGETQADRGCLGWLDTQEPGSVLYVSFGSLAKMDPHEFVELAWGLALSKRPFVWVVRPKLIRGFESGELPDGLGEELRGRGVIVSWAPQEEVLAHPAVGAFFTHSGWNSTVEAIAEGVPMICHPLHGDQYGNARYVADVWRVGVEVDGSHRLERGSIKAAIGRMMESGEGREIGERMKALKMAAEDGIGERGSSHTHLSDLVALIKSF

SEQ ID NO: 46 - protein, 456 aa, Artificial Sequence
  Mutated Wheat Bx8 protein sequence
MAGAGRRRVVFFPFPFLGHFNPVLRLAGALHARGLAVTVFHTEQRVPDPADYPAGYRFVPLPVEVPPELAASEDIARMGMAMNDAAEAPFRDRLAALLAEEAGEDGGVLCVITDVVWYSAQAVARELGVPALGIMTASAAIFRVYMAYQTLIDKAYLPVQDARKDDPVEELPPYLVKDLLRHDTSKLEDFAELLRHTVAGARQSSGLIINTLGAIEAANLERIREDLSVPVFAVAPLHKLAPSAKSSSLGETQADRGCLGWLDTQEPGSVLYVSFGSFAAMDPHEFVELAWGLALSKRPFVWVVRPKLIRGFESGELPDGLGEELRGRGVIVSWAPQEEVLAHPAVGAFFTHSGWNSTVEAIAEGVPMICHPLHGDQYGNARYVADVWRVGVEVDGSHRLERGSIKAAIGRMMESGEGREIGERMKALKMAAEDGIGERGSSHTHLSDLVALIKSF

SEQ ID NO: 47 - protein, 456 aa, Artificial Sequence
  Mutated Wheat Bx8 protein sequence
MAGAGRRRVVFFPFPFLGHFNPVLRLAGALHARGLAVTVFHTEQRVPDPADYPAGYRFVPLPVEVPPELAASEDIARMGMAMNDAAEAPFRDRLAALLAEEAGEDGGVLCVITDVVWYSAQAVARELGVPALGIMTASAAIFRVYMAYQTLIDKAYLPVQDARKDDPVEELPPYLVKDLLRHDTSKLEDFAELLRHTVAGARQSSGLIINTLGAIEAANLERIREDLSVPVFAVAPLHKLAPSAKSSSLGETQADRGCLGWLDTQEPGSVLYVSFGSFAAMDPHEFVELAWGLALSKRPFVWVVRPKLIRGFESGELPDGLGEELRGRGVIVKWAPQEEVLAHPAVGAFFTHSGWNSTVEAIAEGVPMICHPLHGDQYGNARYVADVWRVGVEVDGSHRLERGSIKAAIGRMMESGEGREIGERMKALKMAAEDGIGERGSSHTHLSDLVALIKSF

SEQ ID NO: 48 - protein, 462 aa, Artificial Sequence
  Mutated Wheat Bx8 protein sequences
MAGAGRRRVVFFPFPFLGHFNPVLRLAGALHARGLAVTVFHTEQRVPDPADYPAGYRFVPLPVEVPPELAASEDIARMGMAMNDAAEAPFRDRLAALLAEEAGEDGGVLCVITDVVWYSAQAVARELGVPALGIMTASAAIFRVYMAYQTLIDKAYLPVQDARKDDPVEELPPYLVKDLLRHDTSKLEDFAELLRHTVAGARQSSGLIINTLGAIEAANLERIREDLSVPVFAVAPLHKLAPSAKSSSLGETQADRGCLGWLDTQEPGSVLYVSFGSFAAMDPHEFVELAWGLALSKRPFVWVVRPKLIRGFESGELPDGLGEELRGRGVIVKWAPQEEVLAHPAVGAFFTHSGWNSTVEAIAEGVPMICHPLHGDQYGNARYVADVWRVGVEVDGSHRLERGSIKAAIGRMMESGEGREIGERMKALKMAAEDGIGIGVDVDERGSSHTHLSDLVALIKSF

SEQ ID NO: 49 - protein, 466 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 50 - protein, 466 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 51 - protein, 466 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 52 - protein, 466 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTELVGEQLERGQVKAAIDRLFGTKEGEEIKERMKEFKIAAAKGIGIGVDVDETASPRTDLTDLVDLIKSF

SEQ ID NO: 53 - protein, 467 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAAAKGIGIGVDVDETASPRTDLTDLVDLIKSF

SEQ ID NO: 54 - protein, 455 aa, Artificial Sequence
  Hybrid sequence between Zea mays Bx8 and Bx9 sequences
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 55 - protein, 459 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGMMTASAASLRDYMAYRTLIDKGYLPVREERKDDAVAELPPYRVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 56 - protein, 459 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVKEERKEDPVPELPPYLVKDLLRHETCDLEEFADLLGRVIAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 57 - protein, 459 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARRASGLIFNTFPLIETDTLAEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 58 - protein, 459 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRHETCDLEEFADLLGRVIAAARLSSGLIFHTFPFIEAGTLGEIRDDMSVPVYAVAPLNKLVPTATASLHGVVQADRGCLQWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 59 - protein, 459 aa, Artificial Sequence
  Hybrid sequence between Zea mays Bx8 and Bx9 sequences
MAASCGGRVVVFPFPFQGHFNPVMRLARALHARGVGITVFHTAGARAPDPADYPADYRFVPVPVEVAPELMASEDIAAIVTALNAACEAPFRDRLSALLSAADGEAGEAGGRVRCVLTDVSWDAVLSAARGLGVPALGVMTASAATFRVYMAYRTLVDKGYLPVREERKDDAVAELPPYRVKDLLRVDTSDLEEFAELLARTVAAARLSSGLIFHTFPFIEAGTLGEIRDDMSVPVYAVAPLNKLVPAATASLHGEVQADRGCLRWLDAQRARSVLYVSFGSMAAMDPHEFVELAWGLADAGRPFVWVVRPNLIRGFESGALPDGVEDRVRGRGVVVSWAPQEEVLAHPAVGGFFTHCGWNSTVEAVSEGVPMICHPRHGDQYGNARYVCHVWKVGTEVAGDQLERGEIKAAIDRLMGGSEEGEGIRKRMNELKIAADKGIDESAGSDLTNLVHLINSY

SEQ ID NO: 60 - protein, 7 aa, Artificial Sequence
  Bx-type UDP-glucosyl transferase amino acid motif
  VARIANT (2)..(2): Xaa = L,M,I or F
  VARIANT (3)..(3): Xaa = P or A
  VARIANT (5)..(5): Xaa = Q,L,P,H
  VARIANT (5)..(5)
PXXYXGH

SEQ ID NO: 61 - protein, 7 aa, Artificial Sequence
  Bx-type UDP-glucosyl transferase amino acid motif
  VARIANT (5)..(5): Xaa = Q or L
PFPYXGH

SEQ ID NO: 62 - protein, 7 aa, Artificial Sequence
  Bx-type UDP-glucosyl transferase amino acid motif
PFPYQGH

SEQ ID NO: 63 - protein, 7 aa, Artificial Sequence
  Bx-type UDP-glucosyl transferase amino acid motif
  VARIANT (2)..(2): Xaa = L,M,I or F
  VARIANT (3)..(3): Xaa = P or A
  VARIANT (4)..(4): Xaa = F or Y
  VARIANT (5)..(5): Xaa = any amino acid
PXXXXGH

SEQ ID NO: 64 - protein, 7 aa, Artificial Sequence
  Bx-type UDP glucosyl transferase amino acid motif
  VARIANT (5)..(5): Xaa = any amino acid
PFPFXGH

SEQ ID NO: 65 - protein, 5 aa, Artificial Sequence
  Bx-type UDP glucosyl-transferase amino acid motif
  VARIANT (2)..(2): Xaa = E,D,K,G
  VARIANT (4)..(4): Xaa = any amino acid
SXDXA

SEQ ID NO: 66 - protein, 6 aa, Artificial Sequence
  Bx-type UDP glucosyl-transferase amino acid motif
  VARIANT (5)..(5): Xaa = any amino acid
ASEDXA

SEQ ID NO: 67 - protein, 5 aa, Artificial Sequence
  Bx-type UDP glucosyl-transferase amino acid motif
  VARIANT (2)..(2): Xaa = E,D,K or G
  VARIANT (4)..(4): Xaa = I or A
  VARIANT (5)..(5): Xaa = any amino acid
SXDXX

SEQ ID NO: 68 - protein, 6 aa, Artificial Sequence
  Bx-type UDP glucosyl-transferase amino acid motif
  VARIANT (6)..(6): Xaa = any amino acid
ASEDIX

SEQ ID NO: 69 - protein, 7 aa, Artificial Sequence
  Bx-type UDP glucosyl-transferase amino acid motif
  VARIANT (1)..(1): Xaa = L,M,V or I
  VARIANT (2)..(2): Xaa = any amino acid
  VARIANT (3)..(3): Xaa = A,D,R,V,E,K or G
  VARIANT (4)..(4): Xaa = S,A,T or N
  VARIANT (5)..(5): Xaa = S,C,A,F or M
  VARIANT (6)..(6): Xaa = D,E or A
  VARIANT (7)..(7): Xaa = S,A,E or G
XXXXXXX

SEQ ID NO: 70 - protein, 7 aa, Artificial Sequence
  Bx-type UDP glucosyl-transferase amino acid motif
  VARIANT (1)..(1): Xaa = L or M
  VARIANT (2)..(2): Xaa = any amino acid
  VARIANT (3)..(3): Xaa = A or D
  VARIANT (4)..(4): Xaa = S or A
  VARIANT (5)..(5): Xaa = S,C or A
  VARIANT (6)..(6): Xaa = D or E
XXXXXXA

SEQ ID NO: 71 - protein, 7 aa, Artificial Sequence
  Bx-type UDP glucosyl-transferase amino acid motif
  VARIANT (2)..(2): Xaa = any amino acid
  VARIANT (4)..(4): Xaa = S or A
  VARIANT (6)..(6): Xaa = D or E
LXAXCXA

SEQ ID NO: 72 - protein, 8 aa, Artificial Sequence
  Bx-type UDP glucosyl-transferase amino acid motif
  VARIANT (1)..(1): Xaa = C,F or V
  VARIANT (2)..(2): Xaa = L,I or V
  VARIANT (3)..(3): Xaa = F,L,I or V
  VARIANT (4)..(4): Xaa = A,S,T,F,I or V
  VARIANT (6)..(6): Xaa = A,S,T,G or V
  VARIANT (7)..(7): Xaa = any amino acid
  VARIANT (8)..(8): Xaa = W or L
XXXXDXXX

SEQ ID NO: 73 - protein, 8 aa, Artificial Sequence
  Bx-type UDP glucosyl-transferase amino acid motif
  VARIANT (3)..(3): Xaa = F, L or I
  VARIANT (7)..(7): Xaa = any amino acid
CVXTDVXW

SEQ ID NO: 74 - protein, 11 aa, Artificial Sequence
  Bx-type UDP glucosyl-transferase amino acid motif
  VARIANT (1)..(1): Xaa = P,R,K or A
  VARIANT (2)..(2): Xaa = S,L,T,V or A
  VARIANT (3)..(3): Xaa = L or M
  VARIANT (4)..(4): Xaa = G,P,L or V
  VARIANT (5)..(5): Xaa = M,V,I or L
  VARIANT (6)..(6): Xaa = any amino acid
  VARIANT (7)..(7): Xaa = L,P or T
  VARIANT (8)..(8): Xaa = S,N,T or A
XXXXXXXXSAA

SEQ ID NO: 75 - protein, 11 aa, Artificial Sequence
  Bx-type UDP glucosyl-transferase amino acid motif
  VARIANT (5)..(5): Xaa = M,V or I
  VARIANT (6)..(6): Xaa = any amino acid
PALGXXTASAA

SEQ ID NO: 76 - protein, 11 aa, Artificial Sequence
  Bx-type UDP glucosyl-transferase amino acid motif
  VARIANT (1)..(1): Xaa = P,R,K or A
  VARIANT (2)..(2): Xaa = S,L,T,V or A
  VARIANT (3)..(3): Xaa = L or M
  VARIANT (4)..(4): Xaa = G,P,L or V
  VARIANT (5)..(5): Xaa = M,V,I or L
  VARIANT (6)..(6): Xaa = F,R or M
  VARIANT (7)..(7): Xaa = L,P or T
  VARIANT (8)..(8): Xaa = any amino acid
XXXXXXXXSAA

SEQ ID NO: 77 - protein, 11 aa, Artificial Sequence
  Bx-type UDP glucosyl-transferase amino acid motif
  VARIANT (5)..(5): Xaa = M,V or I
  VARIANT (8)..(8): Xaa = any amino acid
PALGXMTXSAA

SEQ ID NO: 78 - protein, 9 aa, Artificial Sequence
  Bx-type UDP glucosyl transferase amino acid motif
  VARIANT (1)..(1): Xaa = A,V or E
  VARIANT (2)..(2): Xaa = F,T or Y
  VARIANT (3)..(3): Xaa = R,Q or P
  VARIANT (4)..(4): Xaa = A,R,MS,L or T
  VARIANT (6)..(6): Xaa = any amino acid
  VARIANT (7)..(7): Xaa = D,E,A,Q,R or K
  VARIANT (8)..(8): Xaa = N,R,Q,A or K
  VARIANT (9)..(9): Xaa = G,A or C
XXXXLXXXX

SEQ ID NO: 79 - protein, 9 aa, Artificial Sequence
  Bx-type UDP glucosyl transferase amino acid motif
  VARIANT (3)..(3): Xaa = R or Q
  VARIANT (6)..(6): Xaa = any amino acid
  VARIANT (9)..(9): Xaa = G or A
AYXTLXDKX

SEQ ID NO: 80 - protein, 7 aa, Artificial Sequence
  Bx-type UDP glucosyl transferase amino acid motif
  VARIANT (1)..(1): Xaa = A,E or L
  VARIANT (2)..(2): Xaa = E,D or L
  VARIANT (3)..(3): Xaa = F or Y
  VARIANT (5)..(5): Xaa = any amino acid
XXXAXLL

SEQ ID NO: 81 - protein, 7 aa, Artificial Sequence
  Bx-type UDP glucosyl transferase amino acid motif
  VARIANT (2)..(2): Xaa = E or D
  VARIANT (5)..(5): Xaa = any amino acid
EXFAXLL

SEQ ID NO: 82 - protein, 9 aa, Artificial Sequence
  Bx-type UDP glucosyl transferase amino acid motif
  VARIANT (1)..(1): Xaa = M,I or L
  VARIANT (2)..(2): Xaa = G or E
  VARIANT (3)..(3): Xaa = T,A,G,D,Q,R or P
  VARIANT (4)..(4): Xaa = D,G,A,S,T,V or N
  misc_feature (5)..(5): Xaa can be any naturally occurring amino acid
  VARIANT (6)..(6): Xaa = I,V or L
  VARIANT (7)..(7): Xaa = A,G,E,D,Q,N,R or C
  VARIANT (8)..(8): Xaa = Q,R,E,N,K or D
  VARIANT (9)..(9): Xaa = I or L
XXXXXXXXX

SEQ ID NO: 83 - protein, 9 aa, Artificial Sequence
  Bx-type UDP glucosyl transferase amino acid motif
  VARIANT (3)..(3): Xaa = T or A
  VARIANT (4)..(4): Xaa = D,G or A
  VARIANT (5)..(5): Xaa = any amino acid
  VARIANT (7)..(7): Xaa = A,G or E
  VARIANT (8)..(8): Xaa = Q,R or E
IEXXXLXXI

SEQ ID NO: 84 - protein, 9 aa, Artificial Sequence
  Bx-type UDP glucosyl transferase amino acid motif
  VARIANT (3)..(3): Xaa = T or A
  VARIANT (4)..(4): Xaa = D or G
  VARIANT (5)..(5): Xaa = any amino acid
  VARIANT (7)..(7): Xaa = A,G or E
  VARIANT (8)..(8): Xaa = Q,R or E
IEXXXLXEI

SEQ ID NO: 85 - protein, 11 aa, Artificial Sequence
  Bx-type UDP glucosyl transferase amino acid motif
  VARIANT (2)..(2): Xaa = L or I
  VARIANT (3)..(3): Xaa = Y or F
  VARIANT (4)..(4): Xaa = I,A or V
  VARIANT (6)..(6): Xaa = L,I or F
  VARIANT (8)..(8): Xaa = T or S
  VARIANT (9)..(9): Xaa = any amino acid
  VARIANT (10)..(10): Xaa = A or V
  VARIANT (11)..(11): Xaa = S,N,T,G or A
VXXXSXGXXXX

SEQ ID NO: 86 - protein, 11 aa, Artificial Sequence
  Bx-type UDP glucosyl transferase amino acid motif
  VARIANT (9)..(9): Xaa = any amino acid
VLYVSFGSXAA

SEQ ID NO: 87 - protein, 11 aa, Artificial Sequence
  Bx-type UDP glucosyl transferase amino acid motif
  VARIANT (2)..(2): Xaa = L or I
  VARIANT (3)..(3): Xaa = Y or F
  VARIANT (4)..(4): Xaa = I,A or V
  VARIANT (6)..(6): Xaa = L,I or F
  VARIANT (8)..(8): Xaa = T or S
  VARIANT (9)..(9): Xaa = M,L,I or V
  VARIANT (10)..(10): Xaa = A or V
  VARIANT (11)..(11): Xaa = any amino acid
VXXXSXGXXXX

SEQ ID NO: 88 - protein, 11 aa, Artificial Sequence
  Bx-type UDP glucosyl transferase amino acid motif
  VARIANT (11)..(11): Xaa = any amino acid
VLVYSFGSMAX

SEQ ID NO: 89 - protein, 11 aa, Artificial Sequence
  Bx-type UDP glucosyl transferase amino acid motif
  VARIANT (1)..(1): Xaa = V or I
  VARIANT (2)..(2): Xaa = V or I
  VARIANT (3)..(3): Xaa = any amino acid
  VARIANT (8)..(8): Xaa = E,Q or D
  VARIANT (9)..(9): Xaa = E,K or D
  VARIANT (10)..(10): Xaa = V or A
XXXWAPQXXXL

SEQ ID NO: 90 - protein, 11 aa, Artificial Sequence
  Bx-type UDP glucosyl transferase amino acid motif
  VARIANT (1)..(1): Xaa = V or I
  VARIANT (3)..(3): Xaa = any amino acid
XVXWAPQEEVL

SEQ ID NO: 91 - protein, 14 aa, Artificial Sequence
  Bx-type UDP glucosyl transferase amino acid motif
  VARIANT (5)..(5): Xaa = A,M or T
  VARIANT (6)..(6): Xaa = V,I,M,L,T or A
  VARIANT (8)..(8): Xaa = A,S or G
  VARIANT (9)..(9): Xaa = any amino acid
  VARIANT (10)..(10): Xaa = S,A,L,C or G
  VARIANT (11)..(11): Xaa = E,Q,R,G,A or D
  VARIANT (12)..(12): Xaa = T or G
  VARIANT (13)..(13): Xaa = V,H or L
GWNSXXEXXXXXXP

SEQ ID NO: 92 - protein, 9 aa, Artificial Sequence
  Bx-type UDP glucosyl transferase amino acid motif
  VARIANT (5)..(5): Xaa = any amino acid
  VARIANT (6)..(6): Xaa = S or A
TVEAXXEGV

SEQ ID NO: 93 - protein, 7 aa, Artificial Sequence
  Bx-type UDP glucosyl transferase amino acid motif
  VARIANT (1)..(1): Xaa = E,Q,R,G,A or D
  VARIANT (2)..(2): Xaa = T or G
  VARIANT (3)..(3): Xaa = V,H or L
  VARIANT (5)..(5): Xaa = M or V
  VARIANT (6)..(6): Xaa = any amino acid
  VARIANT (7)..(7): Xaa = C,A or S
XXXPXXX

SEQ ID NO: 94 - protein, 7 aa, Artificial Sequence
  Bx-type UDP glucosyl transferase amino acid motif
  VARIANT (6)..(6): Xaa = any amino acid
EGVPMXC

SEQ ID NO: 95 - protein, 8 aa, Artificial Sequence
  Bx-type UDP glucosyl transferase amino acid motif
  VARIANT (1)..(1): Xaa = C or S
  VARIANT (2)..(2): Xaa = C,H,R,L or K
  VARIANT (4)..(4): Xaa = R,L,F,C,S,Y,H or Q
  VARIANT (5)..(5): Xaa = H,G,F or S
  VARIANT (6)..(6): Xaa = any amino acid
XXPXXXDQ

SEQ ID NO: 96 - protein, 8 aa, Artificial Sequence
  Bx-type UDP glucosyl transferase amino acid motif
  VARIANT (2)..(2): Xaa = C or H
  VARIANT (4)..(4): Xaa = R or L
  VARIANT (6)..(6): Xaa = any amino acid
CXPXHXDQ

SEQ ID NO: 97 - protein, 7 aa, Artificial Sequence
  Bx-type UDP glucosyl transferase amino acid motif
  VARIANT (2)..(2): Xaa = I or M
  VARIANT (4)..(4): Xaa = any amino acid
  VARIANT (5)..(5): Xaa = A,D or E
  VARIANT (6)..(6): Xaa = K or D
KXAXXXG

SEQ ID NO: 98 - protein, 7 aa, Artificial Sequence
  Bx-type UDP glucosyl transferase amino acid motif
  VARIANT (4)..(4): Xaa = any amino acid
  VARIANT (5)..(5): Xaa = A or D
KIAXXKG

SEQ ID NO: 99 - protein, 13 aa, Artificial Sequence
  Bx-type UDP glucosyl transferase amino acid motif
  VARIANT (1)..(1): Xaa = R,K or G
  VARIANT (2)..(2): Xaa = A,M,I,V or S
  VARIANT (3)..(3): Xaa = E,K,M,L,I,R,G,S,N or H
  VARIANT (4)..(4): Xaa = E,N,G,D,A,H,V,K,S,Q or I
  VARIANT (5)..(5): Xaa = L,F or M
  VARIANT (6)..(6): Xaa = K,G,R,Q,E or M
  VARIANT (7)..(7): Xaa = S,D,E,Q,G,K,L,N,H,I or M
  VARIANT (8)..(8): Xaa = R,A,K,V,E,M,I,Q or S
  VARIANT (9)..(9): Xaa = A,V,S or M
  VARIANT (10)..(10): Xaa = A,D,E,G,T,S,V,K,E,L,I,Y,R or N
  VARIANT (11)..(11): Xaa = K,R,L,V,F,Q,S,D,E or A
  VARIANT (12)..(12): Xaa = G,C,S,A or T
  VARIANT (13)..(13): Xaa = I,T,A,L,V,F,M or S
XXXXXXXXXXXXX

SEQ ID NO: 100 - protein, 13 aa, Artificial Sequence
  Bx-type UDP glucosyl transferase amino acid motif
  VARIANT (2)..(2): Xaa = A or M
  VARIANT (3)..(3): Xaa = K,M,L,I,R,G,S,N or H
  VARIANT (4)..(4): Xaa = E,N,G,D,A,H or I
  VARIANT (5)..(5): Xaa = L,F or M
  VARIANT (6)..(6): Xaa = K,G,R or Q
  VARIANT (7)..(7): Xaa = S,D,E,Q,G,K,L,N,H,I or M
  VARIANT (8)..(8): Xaa = R,A,K,V,E,M,I or S
  VARIANT (9)..(9): Xaa = A,V,S or M
  VARIANT (10)..(10): Xaa = A,D,E,G,T,S,V,K,E,L or I
  VARIANT (11)..(11): Xaa = K,R,Q,S,D,E or A
  VARIANT (12)..(12): Xaa = G,C,S,A or T
  VARIANT (13)..(13): Xaa = I,T,A,L,V,M  or S
RXXXXXXXXXXXX

SEQ ID NO: 101 - protein, 13 aa, Artificial Sequence
  Bx-type UDP glucosyl transferase amino acid motif
  VARIANT (2)..(2): Xaa = A or M
  VARIANT (3)..(3): Xaa = K,M,L,I,G,N or H
  VARIANT (4)..(4): Xaa = E,N,G,A,D or H
  VARIANT (5)..(5): Xaa = L or M
  VARIANT (6)..(6): Xaa = K,G,R or Q
  VARIANT (7)..(7): Xaa = S,D,E,Q,G,K,L,N,H,I or M
  VARIANT (8)..(8): Xaa = R,A,K,V,E,M or I
  VARIANT (9)..(9): Xaa = A or V
  VARIANT (10)..(10): Xaa = A,D,E,G,S,V or L
  VARIANT (11)..(11): Xaa = K,R,Q,D or E
  VARIANT (12)..(12): Xaa = G,C,S or A
  VARIANT (13)..(13): Xaa = I,T,A or V
RXXXXXXXXXXXX

SEQ ID NO: 102 - protein, 5 aa, Artificial Sequence
  Bx-type UDP glucosyl transferase amino acid motif
GIGVD

SEQ ID NO: 103 - protein, 6 aa, Artificial Sequence
  Bx-type UDP glucosyl transferase amino acid motif
GIGVDV

SEQ ID NO: 104 - protein, 7 aa, Artificial Sequence
  Bx-type UDP glucosyl transferase amino acid motif
GIGVDVD

SEQ ID NO: 105 - protein, 8 aa, Artificial Sequence
  Bx-type UDP glucosyl transferase amino acid motif
GIGVDVDE

SEQ ID NO: 106 - protein, 4 aa, Artificial Sequence
  Bx-type UDP glucosyl transferase amino acid motif
  misc_feature (1)..(2): Xaa can be any naturally occurring amino acid
  misc_feature (4)..(4): Xaa can be any naturally occurring amino acid
XXDX

SEQ ID NO: 107 - protein, 7 aa, Artificial Sequence
  Bx-type UDP glucosyl transferase amino acid motif
  VARIANT (1)..(1): Xaa = P or T
  VARIANT (2)..(2): Xaa = F,L,M,A or I
  VARIANT (3)..(3): Xaa = P or A
  VARIANT (4)..(4): Xaa = F,Y,L or A
  VARIANT (5)..(5): Xaa = Q,L or P
XXXXXGH

SEQ ID NO: 108 - protein, 5 aa, Artificial Sequence
  Bx-type UDP glucosyl transferase amino acid motif
  VARIANT (2)..(2): Xaa = W or R
  VARIANT (3)..(3): Xaa = G,A or S
  VARIANT (4)..(4): Xaa = L or I
AXXXA

SEQ ID NO: 109 - DNA, 70 bp, Tobacco mosaic virus
tatttttacaacaattaccaacaacaacaaacaacaaacaacattacaattactatttacaattacacat

SEQ ID NO: 110 - DNA, 744 bp, Artificial Sequence
  Double 35S promoter DNA sequence
aacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcttacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 111 - DNA, 255 bp, Artificial Sequence
  Nos-terminator DNA sequence
cgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcg

SEQ ID NO: 112 - protein, 462 aa, Artificial Sequence
  Mutated Zea mays Bx9  protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAAMDPHEFVELAWGLADSKRPFVWVVRPNLIRGFESGALPDGVEDEVRGRGIVVAWAPQEEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 113 - protein, 462 aa, Artificial Sequence
  Mutated Zea mays Bx9  protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAAMDPHEFVELAWGLADSKRPFVWVVRPNLIRGFESGALPDGVEDEVRGRGIVVAWAPQAEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 114 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAAMDPHEFVELAWGLADSKRPFVWVVRPNLIRGFESGALPDGVEDEVRGRGIVVAWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 115 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAAMDPHEFVELAWGLADSKRPFVWVVRPNLIRGFESGALPDGVEDEVRGRGIVVAWAPQEEVLAHPAVGGFLTHNGWNSTVEAISEGVPMVCCPRYGDQFGNMRYVCDVWKVGTELVGEQLERGQVKAAIDRLFGTKEGEEIKERMKEFNIAAAKGIGIGVDVDETASPRTDLTDLVDLIKSF

SEQ ID NO: 116 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAAMDPHEFVELAWGLADSKRPFVWVVRPNLIRGFESGALPDGVEDEVRGRGIVVNWAPQEEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 117 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGFESGALPDGVEDEVRGRGIVVAWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 118 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAAMDPHEFVELAWGLADSKRPFVWVVRPNLIRGFESGALPDGVEDEVRGRGIVVKWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 119 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGFESGALPDGVEDEVRGRGIVVKWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 120 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGKESGALPDGVEDEVRGRGIVVAWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 121 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGFESGALPDGVEDEVRGRGIVVNWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 122 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGKESGALPDGVEDEVRGRGIVVNWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 123 - protein, 462 aa, Artificial Sequence; identical to SEQ ID NO: 122
  Mutated Zea mays Bx9 protein sequence

SEQ ID NO: 124 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGKESGALPDGVEDEVRGRGIVVNWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFSIAAAKGIGIGVDVDETASPRTDLTDLVDLIKSF

SEQ ID NO: 125 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGKESGALPDGVEDEVRGRGIVVNWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFNIAAAKGIGIGVDVDETASPRTDLTDLVDLIKSF

SEQ ID NO: 126 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAKMDPHEFVELAWGLADSKRPFVWVVRPNLIRGKESGALPDGVEDEVRGRGIVVNWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFKIAPAKGIGIGVDVDETASPRTDLTDLVDLIKSF

SEQ ID NO: 127 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVSWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSFAKMDPHEFVELAWGLADSKRPFVWVVRPNLIRGKESGALPDGVEDEVRGRGIVVNWAPQVEVLAHPAVGGFLTHNGWNSTVEAISEGVPMVCCPRYGDQFGNMRYVCDVWKVGTELVGEQLERGQVKAAIDRLFGTKEGEEIKERMKEFSIAPAKGIGIGVDVDETASPRTDLTDLVDLIKSF

SEQ ID NO: 128 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAAMDPHEFVELAWGLADSKRPFVWVVRPNLIRGFESGALPDGVEDEVRGRGIVVAWAPQVEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 129 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGFESGALPDGVEDEVRGRGIVVAWAPQVEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 130 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGFESGALPDGVEDEVRGRGIVVKWAPQVEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 131 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGFESGALPDGVEDEVRGRGIVVNWAPQVEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 132 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGKESGALPDGVEDEVRGRGIVVKWAPQVEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 133 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGKESGALPDGVEDEVRGRGIVVNWAPQVEVLAHPAVGGFLTHNGWNSTVEAISEGVPMVCCPRHGDQFGNMRYVCDVWKVGTELVGEQLERGQVKAAIDRLFGTKEGEEIKERMKEFKIAAAKGIGIGVDVDETASPRTDLTDLVDLIKSF

SEQ ID NO: 134 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGKESGALPDGVEDEVRGRGIVVNWAPQVEVLAHPAVGGFLTHNGWNSTVEAISEGVPMVCCPRHGDQFGNMRYVCDVWKVGTELVGEQLERGQVKAAIDRLFGTKEGEEIKERMKEFSIAAAKGIGIGVDVDETASPRTDLTDLVDLIKSF

SEQ ID NO: 135 - protein, 462 aa, Artificial Sequence
  Mutated Zea mays Bx9 protein sequence
MASSRTGAGAGGRVVVFPFPFQGHFNPVMRLARALHARGLAITVFHSGALDPADYPADYRFVPVTVEADPKLLASEDIAAIVTTLNASCDAPFRARLSALLAAEGRDSVRCVFTDVGWNAVLTASSDLGVPALGMMTASAASLRDYMAYRTLIDKGYLPVKEERKEDPVPELPPYLVKDLLRVDTSDLEEFAELLARTVTAARRASGLIFNTFPLIETDTLAEIHKALSVPVFAVAPLNKLVPTATASLHGVVQADRGCLQWLDTQQPGSVLYVSFGSWAKMDPHEFVELAWGLADSKRPFVWVVRPNLIRGKESGALPDGVEDEVRGRGIVVNWAPQVEVLAHPAVGGFLTHNGWNSTVEAISEGVPMVCCPRHGDQFGNMRYVCDVWKVGTELVGEQLERGQVKAAIDRLFGTKEGEEIKERMKEFNIAPAKGIGIGVDVDETASPRTDLTDLVDLIKSF

SEQ ID NO: 136 - DNA, 23 bp, Zea mays
acttgccaattgccatatagagg

SEQ ID NO: 137 - DNA, 23 bp, Zea mays
aatcctcgctcgctcacgctcgg

SEQ ID NO: 138 - DNA, 23 bp, Zea mays
ccgcacggatttaaccgatttgg

SEQ ID NO: 139 - DNA, 23 bp, Zea mays
acacaacaccgtcaggaacgtgg

SEQ ID NO: 140 - DNA, 20 bp, Artificial Sequence
  Designed sequence
acttgccaattgccatatag

SEQ ID NO: 141 - DNA, 20 bp, Artificial Sequence
  Designed sequence
aatcctcgctcgctcacgct

SEQ ID NO: 142 - DNA, 20 bp, Artificial Sequence
  Designed sequence
ccgcacggatttaaccgatt

SEQ ID NO: 143 - DNA, 20 bp, Artificial Sequence
  Designed sequence
acacaacaccgtcaggaacg

SEQ ID NO: 144 - DNA, 106 bp, Artificial Sequence
  Designed guide sequence
aacttgccaattgccatataggttttagagctagaaatagcaagttaaaataaggctagtccgttatcaacttgaaaaagtggcaccgagtcggtgcttttttttt

SEQ ID NO: 145 - DNA, 106 bp, Artificial Sequence
  Designed guide sequence
gaatcctcgctcgctcacgctgttttagagctagaaatagcaagttaaaataaggctagtccgttatcaacttgaaaaagtggcaccgagtcggtgcttttttttt

SEQ ID NO: 146 - DNA, 106 bp, Artificial Sequence
  Designed guide sequence
accgcacggatttaaccgattgttttagagctagaaatagcaagttaaaataaggctagtccgttatcaacttgaaaaagtggcaccgagtcggtgcttttttttt

SEQ ID NO: 147 - DNA, 106 bp, Artificial Sequence
  Designed guide sequence
gacacaacaccgtcaggaacggttttagagctagaaatagcaagttaaaataaggctagtccgttatcaacttgaaaaagtggcaccgagtcggtgcttttttttt

SEQ ID NO: 148 - DNA, 1666 bp, Artificial Sequence
  Designed sequence with desired edit
ttggcaagtggacaccgacaggcagcactgtccgtccgcttctctctccccatctccatctgcaatcctcgctcgcccacactcggcagccatggcgtcgtcgcgcaccggagccggagccggcggccgtgtggtggtcttcccgttcccataccagggccacttcaacccggtgatgcggctggcccgcgcgctgcacgcccggggcctcgcgattaccgtcttccacagcggcgccctggacccggccgactaccccgccgactaccgcttcgtgcccgtgaccgtggaggcggacccgaagctgctggcgtccgaggacatcgccgccatcgtcaccacgctgaacgccagctgcgacgcccccttcagggcccgcctctcggcgctgctggccgccgaggggagggacagcgtccggtgcgtcttcaccgacgtcgtgtggaacgccgtgctgacggcgtccagcgacctcggcgtgcccgcgctcggcatgatgacggccagcgccgcctcgttacgcgactacatggcgtaccgcaccttgatcgacaagggctacctgccggtgaaaggtgagtccgtctccgtcttccatccaatcgtagtcggcgaggttattagcagagagagactagattagacttgcttatcatcacatacctgcagaggagcgcaaggaggatcccgtacccgagctacccccgtacctcgtcaaagacctgctccgggtcgacacgtccgacctggaggagttcgccgaagtgctggcccgcaccgtcacagcggcgcggcgcgcctcggggctcatcttcaacaccttcccgctgatcgagacagacacgctggccgagatccacaaggccttgtcggtgccggtgttcgccgtcgccccgctcaacaagctggtgccgacggccacggccagcctgcacggggtggtgcaggcggaccggggctgcctgcagtggctggacacgcagcagccgggctccgtgctgtacgtcagcttcgggagcttcgccaagatggacccgcacgagttcgtggagctcgcgtgggggctcgccgacagcaagcgccccttcgtgtgggtggtcaggcccaatctcatccgcggcttcgagtccggcgcgctgcccgacggggtggaggacgaggtgcgcggccgtggcatcgtcgtcaagtgggcgccgcaggaggaggtgctcgcgcacccggccgtcggcggcttcctcacccacaacggctggaactccaccgtcgaggccatctcggagggcgtgcccatggtctgctgcccgcggcacggggaccagttcggcaacatgaggtacgtgtgcgacgtgtggaaggtaggcacggagctcgtgggggaacagctggagagaggccaggtcaaggccgccatcgacaggctctttggcaccaaggaaggggaggagatcaaggagaggatgaaggaattcaagatcgctgcggccaaaggcatcggcatcggcgtcgacgtcgacgaaactgcgtcgccccgcacggatttaaccgatcttgttgatctcataaaatccttctgagctcatgacgatcgatctgttgggtgatgcctactctgtccatcgtgctcgacttgtcttcctactactgtaccactgtctgtgctcacttt

SEQ ID NO: 149 - DNA, 1584 bp, Artificial Sequence
  Homology region for editing site
tgttttttgtctaaaccggctataaatttatagttcgcttcttctctctttcctcatttctctcctctacataagtatctaacctacttaacgagcaagtttaataatatagcctacaacgagttctataatgttgtcatgtcacatatggccaattaaaagtctattcatataataactctcgtacaatacaagttgtacatttaatatttggtccacctctctctcatatagagagccttggagtccgcgtgcaaccgacacttagtctatttccagtagcttacctactctcatatctatattcaaactctactatgcgaacagtgcacctacagtgcaaaacagtaccaaacaatgttttggatgtctatatgggtaaactgatgaacacggtcttaactagctaccaatgagcatcttgcttttattctctcctcgtttctctttttccacatcaccataaatttgatgtggtatctattgtaacccgtctacatcacttgttagacttgctcttaaggtctgtttgtttagaattataatcagattagattatacaatctaacttattttaaactgaatctgacttatttaaaaataatacttagttcaaaataaattagattatataatttaaatagattataatatcaaacaaagagagcccggaaaaaggagggagccaaaagtgagactacctgcaacgcatcccgcgacggccccccttccctacgcctgtacgcgtttgccgtcactcttcccatgcaacggcaaacactaaacactcccctctgcgctacagttcaagggaggcatacagtggtcctctagatttgaaggagtggttggcagccctctacgctcgatttctgtgtacgttattgtttgttagttgacaaaaaaaactgtaatgaatagttatgggaaaatattttatggttgtagtggggtatagggacagtatttaggaggaaccgctgcgggagatgaaaaagtaggggataaaatgttaatgatgtggcaaaaaaaaatatatgaggaaaaatttagagtaaccgccatatgtgtcgtgtgaggtgcgtgctggctgaccacgtggccaaaatggggcgtggagcggcacaggctgagccaaaaaattgtggtgttatccgctagccgacccgccgctgctgacgtggcgtgcggccataggttagacagcgtcacacccctgcgtggaccgtagcaaggcccgtgggccccacctacgtctccggttggcagaaaccggcgccctctcttcctgccccgttttgacgtttggggatctcgaaacccccgtctcggttcctgttttccccgccgtttggccccgagactgctgcgcccaccaacccggctcacccagccgatgacacgcgggcccactatggtcgggccccgcgtcacacgtaacacgtcaccgctccgcacgtgggaatggaccacccgaccgtgtctgcggcgcctcccttccccccactaataaattactaacacatcgtgggtcccacgcccctctcgcgaaacacgcacgcgccgtccttttctcccctctat

SEQ ID NO: 150 - DNA, 1424 bp, Artificial Sequence
  Homology region for editing site
cacgttcctgacggtgttgtgttctagtgtatcattgtgcgatcagttccatgttcttggccatgttttagaataaaacagaaaacttgtgttcttggtgtaccattgtgctcactttcatgttcttgacggcgagttggaataaaacaagacttgtgtcatggtgtaccattgccatcattttcatgttaatataaagatttgacgcgagacgcaacattcgtaaacacatctatccacatcaaataaattagagacatttgtgagggtagccttctcccaccttacaattatgactttacatctcgtttttttattttttaatcattaattttaattttcactaatagtgttaaaagagatgggtaaacggcaactttgtttttaaaaccgaaagctaaaatcataaagttgagtataattacaattatcgctttttttattagcatgtgtccttgttgagtaccaaaaaaaattcggacggtcatgccgtaggctcagatagtccacgattcaattaactcggatgagagcccttattctgtccgtgtttatacgactaatcacataagaatctgttaggatatatatagagatgggttcagacctctagggtgtgtttggttgtaggacagacaggacagggacgtcccctgacgtcctctcttgtccctctaattttgagggataactggggacaacactggatagtcttatctcaaccattgactttgaaccaaacaaccttatttgagggatcgtcccaccctatctagttctgtcattgcaaccaaacgcatccctagtttcagccttgggatccttctacaagttttctcctccaaatgttatttgacacttggctcaatcattccctgggacttaaggtccttctctctcgcttagcttgtactccctactacaagaacttaggtgtaagtaatataagtcttatttcactctactagaattagagcttcttctcacctaaaaggataaaaaacttatgttaacttgtatcatcatctagatcaaaagcacgcgacattatttcaccgttaagtccgtgtatctaaacactgacactttccttccgacaaggggcgtgtgactttcggtcccgccaacaccaaccatacacccgctaaagtaaatggctaaaattgactaaaaattagtctgcaaaccatacacccgctaaagtaaaacttggaaaggtctagttttgcgtgagccgccaacaccgaccaaactttacatcactgtgacagtttcagccttccggtcccggcgacaggccgacagccatggcggacgcgggggtgacgggggtactggccaagctgggtgagctggcggcggaggaggcgacggcgctgctgcgcgtggacgccgagatccgggcgttgcggcggaagctggcc

SEQ ID NO: 151 - DNA, 52 bp, Artificial Sequence
  Homology region for editing site
gtcccacgcccctctcgcgaaacacgcacgcgccgtccttttctcccctcta

SEQ ID NO: 152 - DNA, 1423 bp, Artificial Sequence
  Homology region for editing site
tcctgacggtgttgtgttctagtgtatcattgtgcgatcagttccatgttcttggccatgttttagaataaaacagaaaacttgtgttcttggtgtaccattgtgctcactttcatgttcttgacggcgagttggaataaaacaagacttgtgtcatggtgtaccattgccatcattttcatgttaatataaagatttgacgcgagacgcaacattcgtaaacacatctatccacatcaaataaattagagacatttgtgagggtagccttctcccaccttacaattatgactttacatctcgtttttttattttttaatcattaattttaattttcactaatagtgttaaaagagatgggtaaacggcaactttgtttttaaaaccgaaagctaaaatcataaagttgagtataattacaattatcgctttttttattagcatgtgtccttgttgagtaccaaaaaaaattcggacggtcatgccgtaggctcagatagtccacgattcaattaactcggatgagagcccttattctgtccgtgtttatacgactaatcacataagaatctgttaggatatatatagagatgggttcagacctctagggtgtgtttggttgtaggacagacaggacagggacgtcccctgacgtcctctcttgtccctctaattttgagggataactggggacaacactggatagtcttatctcaaccattgactttgaaccaaacaaccttatttgagggatcgtcccaccctatctagttctgtcattgcaaccaaacgcatccctagtttcagccttgggatccttctacaagttttctcctccaaatgttatttgacacttggctcaatcattccctgggacttaaggtccttctctctcgcttagcttgtactccctactacaagaacttaggtgtaagtaatataagtcttatttcactctactagaattagagcttcttctcacctaaaaggataaaaaacttatgttaacttgtatcatcatctagatcaaaagcacgcgacattatttcaccgttaagtccgtgtatctaaacactgacactttccttccgacaaggggcgtgtgactttcggtcccgccaacaccaaccatacacccgctaaagtaaatggctaaaattgactaaaaattagtctgcaaaccatacacccgctaaagtaaaacttggaaaggtctagttttgcgtgagccgccaacaccgaccaaactttacatcactgtgacagtttcagccttccggtcccggcgacaggccgacagccatggcggacgcgggggtgacgggggtactggccaagctgggtgagctggcggcggaggaggcgacggcgctgctgcgcgtggacgccgagatccgggcgttgcggcggaagctggcctgca

SEQ ID NO: 153 - DNA, 121 bp, Artificial Sequence
  Homology region for editing site
aatggaccacccgaccgtgtctgcggcgcctcccttccccccactaataaattactaacacatcgtgggtcccacgcccctctcgcgaaacacgcacgcgccgtccttttctcccctctat

SEQ ID NO: 154 - DNA, 111 bp, Artificial Sequence
  Homology region for editing site
cacgttcctgacggtgttgtgttctagtgtatcattgtgcgatcagttccatgttcttggccatgttttagaataaaacagaaaacttgtgttcttggtgtaccattgtgc

SEQ ID NO: 155 - DNA, 1109 bp, Artificial Sequence
  Donor for desired edit
actcggcagccatggcgtcgtcgcgcaccggagccggagccggagccggcggccgtgtggtggtcttcccgttcccgtaccagggccacttcaacccggtgatgcggctggcccgcgcgctgcacgcccggggcctcgcgattaccgtcttccacagcggcgccctggacccggccgactaccccgccgactaccgcttcgtgcccgtgaccgtggaggcggacccgaagctgctggcgtccgaggacatcgccgccatcgtcaccacgctgaacgccagctgcgatgcccccttcagggcccgcctctcggcgctgctggccgccgaggggagggacagcgtccggtgcgtcttcaccgacgtcgtctggaacgccgtgctgacggcgtccagcgacctcggcgtgcccgcgctcggcatgatgacggccagcgccgcctcgttacgcgactacatggcgtaccgcaccttgatcgacaagggctacctgccggtgaaaggtgagtccatctccgtctccatccaatcgtcgtcggcgaggttattagcagagggagactagattagatttgcttatcatcacatacctgcagaggagcgcaaggaggatcccgtacccgagctacccccgtaccgcgtcaaagacctgctccgggtcgacacgtccgacctggaggagttcgccgaagtgctggcccgcaccgtcaccgcggcgcggcgcgcctcggggctcatcttcaacaccttcccgctgatcgagacagacacgctggccgagatccacaaggccttgtcggtgccggtgttcgccgtcgccccgctcaacaagctggtgccgacggccacggccagcctgcacggggtggtccaggcggaccggggctgcctgcagtggctggacacgcagcagccgggctccgtgctgtacgtcagcttcgggagcttcgccaagatggacccgcacgagttcgtggagctcgcgtgggggctcgccgacagcaagcgccccttcgtgtgggtggtcaggcccaatctcatccgcggcttcgagtccggcgcgctgcccgacggggtggaggacgaggtgcgcggccgcggcatcgtcgtc

SEQ ID NO: 156 - DNA, 49 bp, Artificial Sequence
  Homology region for editing site
ctgtccgtccgcttctctctccccatctccatctgcaatcctcgctcgc

SEQ ID NO: 157 - DNA, 42 bp, Artificial Sequence
  Homology region for editing site
agtgggcgccgcaggaggaggtgctcgcgcacccggccgtcg

SEQ ID NO: 158 - DNA, 23 bp, Zea mays
gtacgtcagcttcgggagcatgg

SEQ ID NO: 159 - DNA, 20 bp, Artificial Sequence
  Designed sequence
gtacgtcagcttcgggagca

SEQ ID NO: 160 - DNA, 106 bp, Artificial Sequence
  Designed guide sequence
agtacgtcagcttcgggagcagttttagagctagaaatagcaagttaaaataaggctagtccgttatcaacttgaaaaagtggcaccgagtcggtgcttttttttt

SEQ ID NO: 161 - DNA, 23 bp, Zea mays
gccgcggcatcgtcgtcacctgg

SEQ ID NO: 162 - DNA, 23 bp, Zea mays
aatcctcgctcgctcacgctcgg

SEQ ID NO: 163 - DNA, 20 bp, Artificial Sequence
  Designed sequence
gccgcggcatcgtcgtcacc

SEQ ID NO: 164 - DNA, 20 bp, Artificial Sequence
  Designed sequence
aatcctcgctcgctcacgct

SEQ ID NO: 165 - DNA, 106 bp, Artificial Sequence
  Designed guide sequence
agccgcggcatcgtcgtcaccgttttagagctagaaatagcaagttaaaataaggctagtccgttatcaacttgaaaaagtggcaccgagtcggtgcttttttttt

SEQ ID NO: 166 - DNA, 106 bp, Artificial Sequence; identical to SEQ ID NO: 145
  Designed guide sequence

SEQ ID NO: 167 - DNA, 27 bp, Zea mays
tttcaccggcaggtagcccttgtcgat

SEQ ID NO: 168 - DNA, 23 bp, Artificial Sequence
  Designed sequence
accggcaggtagcccttgtcgat

SEQ ID NO: 169 - DNA, 44 bp, Artificial Sequence
  Designed guide sequence
taatttctactaagtgtagataccggcaggtagcccttgtcgat

SEQ ID NO: 170 - DNA, 27 bp, Zea mays
tttgacgcggtacgggggtagctcggg

SEQ ID NO: 171 - DNA, 23 bp, Artificial Sequence
  Designed sequence
acgcggtacgggggtagctcggg

SEQ ID NO: 172 - DNA, 44 bp, Artificial Sequence
  Designed guide sequence
taatttctactaagtgtagatacgcggtacgggggtagctcggg

SEQ ID NO: 173 - DNA, 27 bp, Zea mays
tttctcccctctatatggcaattggca

SEQ ID NO: 174 - DNA, 23 bp, Artificial Sequence
  Designed sequence
tcccctctatatggcaattggca

SEQ ID NO: 175 - DNA, 44 bp, Artificial Sequence
  Designed guide sequence
taatttctactaagtgtagattcccctctatatggcaattggca

SEQ ID NO: 176 - DNA, 27 bp, Zea mays
tttggcacgaaagaaggggaggagatc

SEQ ID NO: 177 - DNA, 23 bp, Artificial Sequence
  Designed sequence
gcaccaaggaaggggaggagatc

SEQ ID NO: 178 - DNA, 44 bp, Artificial Sequence
  Designed guide sequence
taatttctactaagtgtagatgcaccaaggaaggggaggagatc

SEQ ID NO: 179 - DNA, 1032 bp, Artificial Sequence
  Donor for desired edit
accagggccacttcaacccggtgatgcggctggcccgcgcgctgcacgcccggggcctcgcgattaccgtcttccacagcggcgccctggacccggccgactaccccgccgactaccgcttcgtgcccgtgaccgtggaggcggacccgaagctgctggcgtccgaggacatcgccgccatcgtcaccacgctgaacgccagctgcgatgcccccttcagggcccgcctctcggcgctgctggccgccgaggggagggacagcgtccggtgcgtcttcaccgacgtcgtctggaacgccgtgctgacggcgtccagcgacctcggcgtgcccgcgctcggcatgatgacggccagcgccgcctcgttacgcgactacatggcgtaccgcaccttgatcgacaagggctacctcccagtgaaaggtgagtccatctccgtctccatccaatcgtcgtcggcgaggttattagcagagggagactagattagatttgcttatcatcacatacctgcagaggagcgcaaggaggatcccgtacccgagctacccccataccgggtcaaagacctgctccgggtcgacacgtccgacctggaggagttcgccgaagtgctggcccgcaccgtcaccgcggcgcggcgcgcctcggggctcatcttcaacaccttcccgctgatcgagacagacacgctggccgagatccacaaggccttgtcggtgccggtgttcgccgtcgccccgctcaacaagctggtgccgacggccacggccagcctgcacggggtggtccaggcggaccggggctgcctgcagtggctggacacgcagcagccgggctccgtgctgtacgtcagcttcgggagcttcgccaagatggacccgcacgagttcgtggagctcgcgtgggggctcgccgacagcaagcgccccttcgtgtgggtggtcaggcccaatctcatccgcggcttcgagtccggcgcgctgcccgacggggtggaggacgaggtgcgcggccgcggcatcgtcgtca

SEQ ID NO: 180 - DNA, 162 bp, Artificial Sequence
  Homology region for editing site
ggcaattggcaagtggacaccgacaggcagcactgtccgtccgcttctctctccccatctccatctgcaatcctcgctcgctcacgctcggcagccatggcgtcgtcgcgcaccggagccggagccggagccggcggccgtgtggtggtcttcccgttcccg

SEQ ID NO: 181 - DNA, 63 bp, Artificial Sequence
  Homology region for editing site
tgggcgccgcaggaggaggtgctcgcgcacccggccgtcggcggcttcctcacccacaacggt

SEQ ID NO: 182 - DNA, 83 bp, Artificial Sequence
  Homology region for editing site
gctcacgctcggcagccatggcgtcgtcgcgcaccggagccggagccggagccggcggccgtgtggtggtcttcccgttcccg

SEQ ID NO: 183 - DNA, 56 bp, Artificial Sequence
  Homology region for editing site
tgggcgccgcaggaggaggtgctcgcgcacccggccgtcggcggcttcctcaccca

SEQ ID NO: 184 - DNA, 1440 bp, Artificial Sequence
  Donor for desired edit
atatggcaattggcaagtggacaccgacaggcagcactgtccgtccgcttctctctccccatctccatctgcaatcctcgctcgctcacgctcggcagccatggcgtcgtcgcgcaccggagccggagccggagccggcggccgtgtggtggtcttcccgttcccgtaccagggccacttcaacccggtgatgcggctggcccgcgcgctgcacgcccggggcctcgcgattaccgtcttccacagcggcgccctggacccggccgactaccccgccgactaccgcttcgtgcccgtgaccgtggaggcggacccgaagctgctggcgtccgaggacatcgccgccatcgtcaccacgctgaacgccagctgcgatgcccccttcagggcccgcctctcggcgctgctggccgccgaggggagggacagcgtccggtgcgtcttcaccgacgtcgtctggaacgccgtgctgacggcgtccagcgacctcggcgtgcccgcgctcggcatgatgacggccagcgccgcctcgttacgcgactacatggcgtaccgcaccttgatcgacaagggctacctcccagtgaaaggtgagtccatctccgtctccatccaatcgtcgtcggcgaggttattagcagagggagactagattagatttgcttatcatcacatacctgcagaggagcgcaaggaggatcccgtacccgagctacccccataccgggtcaaagacctgctccgggtcgacacgtccgacctggaggagttcgccgaagtgctggcccgcaccgtcaccgcggcgcggcgcgcctcggggctcatcttcaacaccttcccgctgatcgagacagacacgctggccgagatccacaaggccttgtcggtgccggtgttcgccgtcgccccgctcaacaagctggtgccgacggccacggccagcctgcacggggtggtccaggcggaccggggctgcctgcagtggctggacacgcagcagccgggctccgtgctgtacgtcagcttcgggagcttcgccaagatggacccgcacgagttcgtggagctcgcgtgggggctcgccgacagcaagcgccccttcgtgtgggtggtcaggcccaatctcatccgcggcttcgagtccggcgcgctgcccgacggggtggaggacgaggtgcgcggccgcggcatcgtcgtcaagtgggcgccgcaggaggaggtgctcgcgcacccggccgtcggcggcttcctcacccacaacggttggaactccaccgtcgaggccatctcggagggcgtgcccatggtctgctgcccgcggcacggggaccagttcggcaacatgaggtacgtgtgcgacgtgtggaaggtgggcacggagctcgtgggggaacagctggagagaggccaggtcaaggccgccatcgacaggctctttgg

SEQ ID NO: 185 - DNA, 125 bp, Artificial Sequence
  Homology region for editing site
gaatggaccacccgaccgtgtctgcggcgcctcccttccccccactaataaattactaacacatcgtgggtcccacgcccctctcgcgaaacacgcacgcgccgtccttttctcggctctatatg

SEQ ID NO: 186 - DNA, 35 bp, Artificial Sequence
  Homology region for editing site
aggagaggatgaaggaattcaagatcgctgcggcc